(19) United States
(12) Patent Application Publication
(10) Pub. No.: US 2023/0002431 A1
(43) Pub. Date: Jan. 5, 2023

(54) ORGANOMETALLIC COMPOUND, ORGANIC LIGHT-EMITTING DEVICE INCLUDING ORGANOMETALLIC COMPOUND, AND DIAGNOSTIC COMPOSITION INCLUDING ORGANOMETALLIC COMPOUND

(71) Applicant: SAMSUNG ELECTRONICS CO., LTD., Suwon-si (KR)

(72) Inventors: Banglin LEE, Suwon-si (KR); Kum Hee LEE, Suwon-si (KR); Jongwon CHOI, Yongin-si (KR); Yoonhyun KWAK, Seoul (KR); Hyun KOO, Seongnam-si (KR); Ohyun KWON, Seoul (KR); Jun CHWAE, Seoul (KR)

(21) Appl. No.: 17/861,967

(22) Filed: Jul. 11, 2022

Related U.S. Application Data
(63) Continuation of application No. 15/845,162, filed on Dec. 18, 2017.

(30) Foreign Application Priority Data
Dec. 22, 2016 (KR) 10-2016-0176836

(51) Int. Cl. C07F 15/00; H01L 51/00; H01L 51/50; C09K 11/06

(52) U.S. Cl. CPC C07F 15/0086; C07F 15/006; H01L 51/0087; H01L 51/5012; C09K 11/06; C09K 2211/185; H01L 51/5016

(57) ABSTRACT

An organometallic compound represented by Formula 1:

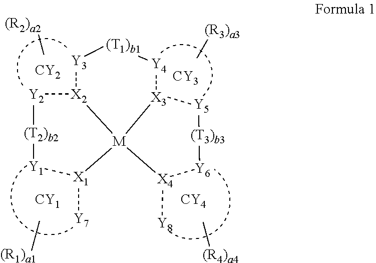

wherein, in Formula 1, groups and variables are the same as described in the specification.

### CROSS-REFERENCE TO RELATED APPLICATION

This continuation application claims priority to U.S. patent application Ser. No. 15/845,162, filed on Dec. 18, 2017, which claims priority to Korean Patent Application No. 10-2016-0176836, filed on Dec. 22, 2016, and all the benefits accruing therefrom under 35 U.S.C. § 119, the contents of which are incorporated herein in their entirety by reference.

### BACKGROUND

### 1. Field

The present disclosure relates to an organometallic compound, an organic light-emitting device including the organometallic compound, and a diagnostic composition that includes the organometallic compound.

### 2. Description of the Related Art

Organic light-emitting devices (OLEDs) are self-emission devices which produce full-color images. In addition, OLEDs have wide viewing angles and exhibit excellent driving voltage and response speed characteristics.

Typical OLEDs include an anode, a cathode, and an organic layer disposed between the anode and the cathode and including an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. The excitons may transit from an excited state to a ground state, thus generating light.

Further, light-emitting compounds, e.g., phosphorescence-emitting compounds, can also be used to monitor, sense, or detect biological materials, including a variety of cells and proteins.

Various types of organic light emitting devices are known. However, there still remains a need in OLEDs having low driving voltage, high efficiency, high brightness, and long lifespan.

### SUMMARY

Provided are an organometallic compound, an organic light-emitting device including the organometallic compound, and a diagnostic composition including the organometallic compound.

Additional aspects will be set forth in part in the description which follows and, in part, will be apparent from the description, or may be learned by practice of the presented embodiments.

According to an aspect of an embodiment, an organometallic compound is represented by Formula 1:

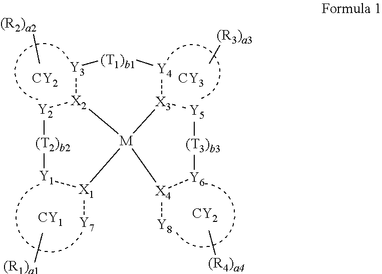

wherein, in Formula 1,

M is selected from platinum (Pt) and palladium (Pd),

X₁ is nitrogen (N),

X₂ to X₄ are each independently selected from carbon (C) and N,

a bond between X₁ and M is a coordinate bond; and one bond selected from a bond between X₂ and M, a bond between X₃ and M, and a bond between X₄ and M is a coordinate bond, while the other two bonds are each a covalent bond,

Y₁ to Y₆ are each independently selected from C and N,

Y₇ and Y₈ are each independently selected from C, N, oxygen (O), and sulfur (S),

a bond between X₁ and Y₇, a bond between X₁ and Y₁, a bond between X₂ and Y₂, a bond between X₂ and Y₃, a bond between X₃ and Y₄, a bond between X₃ and Y₅, a bond between X₄ and Y₆, and a bond between X₄ and Y₈ are each independently selected from a single bond and a double bond,

CY₁ is selected from an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene-5-oxide group, an aza-9H-fluoren-9-one group, and an azadibenzothiophene-5,5-dioxide group, each including at least one N as a ring-forming atom,

CY₂ to CY₄ are each independently selected from a C₅-C₃₀ carbocyclic group and a C₁-C₃₀ heterocyclic group,

T₁ to T₃ are each independently selected from *—N[(L₅)ₐ₅-(R₅)]—*′, *—B(R₅)—*′, *—P(R₅)—*′, *—C(R₅)(R₆)—*′, *—Si(R₅)(R₆)—*′, *—Ge(R₅)(R₆)—*′, *—S—*′, *—Se—*′, *—O—*′, *—C(═O)—*′, *—S(═O)—*′, *—S(═O)₂—*′, *—C(R₅)═*′, *═C(R₅)—*′, *—C(R₅)═C(R₆)—*′, *—C(═S)—*′, and *—C≡C—*′,

L₅ is selected from a single bond, a substituted or unsubstituted C₅-C₃₀ carbocyclic group, and a substituted or unsubstituted C₁-C₃₀ heterocyclic group,

a5 is an integer from 1 to 3; when a5 is 2 or greater, at least two L₅ groups are identical to or different from each other,

R₅ and R₆ are optionally bound via a first linking group to form a substituted or unsubstituted C₅-C₃₀ carbocyclic group or a substituted or unsubstituted C₁-C₃₀ heterocyclic group,

b1 to b3 are each independently selected from 0, 1, 2, and 3; provided that when b1 is 0, *-(T₁)_{b1}-*′ is a single bond; when b2 is 0, *-(T₂)_{b2}-*′ is a single bond; when b3 is 0, *-(T₃)_{b3}-*′ is a single bond,

R₁ to R₆ are each independently selected from hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),

a1 to a4 are each independently selected from 0, 1, 2, 3, 4, and 5,

two of R₁ groups in the number of a1 are optionally bound to form a substituted or unsubstituted C₅-C₃₀ carbocyclic group or a substituted or unsubstituted C₁-C₃₀ heterocyclic group,

two of R₂ groups in the number of a2 are optionally bound to form a substituted or unsubstituted C₅-C₃₀ carbocyclic group or a substituted or unsubstituted C₁-C₃₀ heterocyclic group,

two of R₃ groups in the number of a3 are optionally bound to form a substituted or unsubstituted C₅-C₃₀ carbocyclic group or a substituted or unsubstituted C₁-C₃₀ heterocyclic group,

two of R₄ groups in the number of a4 are optionally bound to form a substituted or unsubstituted C₅-C₃₀ carbocyclic group or a substituted or unsubstituted C₁-C₃₀ heterocyclic group,

at least two of R₁ to R₄, wherein the at least two of R₁ to R₄ are adjacent to each other, are optionally bound to form a substituted or unsubstituted C₅-C₃₀ carbocyclic group or a substituted or unsubstituted C₁-C₃₀ heterocyclic group,

at least one of R₅ and R₆ and at least one of R₁ to R₄ are optionally bound to form a substituted or unsubstituted C₅-C₃₀ carbocyclic group or a substituted or unsubstituted C₁-C₃₀ heterocyclic group,

at least one substituent of the substituted C₅-C₃₀ carbocyclic group, substituted C₁-C₃₀ heterocyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group is selected from:

deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇), and —P(═O)(Q₃₈)(Q₃₉),

wherein Q₁ to Q₉, Q₁₁ to Q₁₉, Q₂₁ to Q₂₉, and Q₃₁ to Q₃₉ are each independently selected from a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ alkyl group, and a C₆-C₆₀ aryl group.

According to an aspect of another embodiment, an organic light-emitting device includes:

a first electrode;

a second electrode; and

an organic layer disposed between the first electrode and the second electrode,

wherein the organic layer includes an emission layer, wherein the organic layer includes at least one organometallic compound described above.

In the emission layer, the organometallic compound serves as a dopant.

According to an aspect of another embodiment, a diagnostic composition includes at least one organometallic compound represented by Formula 1.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic cross-sectional view of an organic light-emitting device according to an embodiment;

FIG. 2 is a graph of intensity (arbitrary unit, a.u.) versus wavelength (nanometers, nm), illustrating an ultraviolet (UV) absorption spectrum and a photoluminescence (PL) spectrum of Compound 5;

FIG. 3 is a graph of intensity (a.u.) versus wavelength (nm) with respect to electroluminescence (EL) of an organic light-emitting device of Example 5;

FIG. 4 is a graph of luminous efficiency (candela per ampere, cd/A) versus luminance (candela per square meter, cd/m²) with respect to organic light-emitting devices of Example 5 and Comparative Example A;

FIG. 5 is a graph of luminous efficiency (cd/A) versus luminance (cd/m²) with respect to the organic light-emitting device of Example 2; and

FIG. 6 is a graph of luminance (percent, %) versus time (hours, hrs) with respect to the organic light-emitting device of Example 2.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein.

Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms (“a”, “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

In an embodiment, an organometallic compound may be represented by Formula 1:

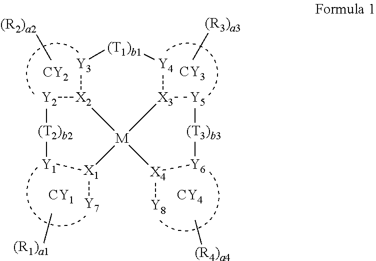

In Formula 1, M may be platinum (Pt) or palladium (Pd).

In some embodiments, in Formula 1, M may be Pt, but embodiments are not limited thereto.

The organometallic compound represented by Formula 1 may be neutral, and may not include ion pairs of cations and anions.

In Formula 1, X₁ may be nitrogen (N), and X₂ to X₄ may each independently be carbon (C) or N.

In Formula 1, a bond between X₁ and M may be a coordinate bond, one bond selected from a bond between X₂ and M; a bond between X₃ and M; and a bond between X₄ and M may be a coordinate bond, while the rest two bonds may each be a covalent bond.

In an embodiment, in Formula 1, X₂ and X₃ may each be C, X₄ may be N, a bond between X₂ and M and a bond between X₃ and M may each be a covalent bond, a bond between X₄ and M may be a coordinate bond, but embodiments are not limited thereto.

In Formula 1, Y₁ to Y₆ may each independently be C or N, and Y₇ and Y₈ may each independently be C, N, oxygen (O), or sulfur (S).

In an embodiment, in Formula 1, Y₁ to Y₅ may each be C, Y₆ to Y₈ may each independently be C or N, but embodiments are not limited thereto.

In Formula 1, a bond between X₁ and Y₇, a bond between X₁ and Y₁, a bond between X₂ and Y₂, a bond between X₂ and Y₃, a bond between X₃ and Y₄, a bond between X₃ and Y₅, a bond between X₄ and Y₆, and a bond between X₄ and Y₈ may each independently be selected from a single bond and a double bond.

In Formula 1, CY₁ may be selected from an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene-5-oxide group, an aza-9H-fluoren-9-one group, and an azadibenzothiophene-5,5-dioxide group, each including at least one N as a ring-forming atom, and CY₂ to CY₄ may each independently be selected from a C₅-C₃₀ carbocyclic group and a C₁-C₃₀ heterocyclic group.

The terms “azacarbazole group”, “azadibenzoborole group”, “azadibenzophosphole group”, “azafluorene group”, “azadibenzosilole group”, “azadibenzogermole group”, “azadibenzothiophene group”, “azadibenzoselenophene group”, “azadibenzofuran group”, “azadibenzothiophene-5-oxide group”, “aza-9H-fluoren-9-one group”, and “azadibenzothiophene-5,5-dioxide group” each refer to a hetero-ring having substantially the same backbone as “a carbazole group”, “a dibenzoborole group”, “a dibenzophosphole group”, “a fluorene group”, “a dibenzosilole group”, “a dibenzogermole group”, “a dibenzothiophene group”, “a dibenzoselenophene group”, “a dibenzofuran group”, “a dibenzothiophene-5-oxide group”, “a 9H-fluoren-9-one group”, and “a dibenzothiophene-5,5-dioxide group”, respectively, in which at least one ring-forming carbon is substituted with N.

In some embodiments, in Formula 1, CY₂ to CY₄ may each independently be selected from a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indene group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene-5,5-dioxide group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene-5-oxide group, an aza-9H-fluoren-9-one group, an azadibenzothiophene-5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, and a 5,6,7,8-tetrahydroquinoline group.

In one or more embodiments, in Formula 1, CY₂ and CY₃ may each be a benzene group, and CY₄ may be an azadibenzofuran group, an azadibenzothiophene group, a pyridine group, a pyrimidine group, a quinoline group, an isoquinoline group, an imidazole group, or a pyrazole group, but embodiments are not limited thereto.

In one or more embodiments, in Formula 1, CY₁ may be identical to CY₄, but embodiments are not limited thereto.

In Formula 1, T₁ to T₃ may each independently be selected from *—N[(L₅)ₐ₅-(R₅)]—*′, *—B(R₅)—*′, *—P(R₅)—*′, *—C(R₅)(R₆)—*′, *—Si(R₅)(R₆)—*′, *—Ge(R₅)(R₆)—*′, *—S—*′, *—Se—*′, *—O—*′, *—C(═O)—*′, *—S(═O)—*′, *—S(═O)₂—*′, *—C(R₅)═*′, *═C(R₅)—*′, *—C(R₅)═C(R₆)—*′, *—C(═S)—*′, and *—C≡C—*′. R₅ and R₆ are the same as those described herein.

L₅ may be selected from a single bond, a substituted or unsubstituted C₅-C₃₀ carbocyclic group, and a substituted or unsubstituted C₁-C₃₀ heterocyclic group, and a5 may be an integer from 1 to 3 (for example, a5 may be 1); when a5 is 2 or greater, at least two L₅ groups may be identical to or different from each other.

In an embodiment, L₅ may be selected from:

a phenylene group, a naphthylene group, a fluorenylene group, a pyridinylene group, a pyrimidinylene group, and a carbazolylene group; and

a phenylene group, a naphthylene group, a fluorenylene group, a pyridinylene group, a pyrimidinylene group, and a carbazolylene group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, a biphenyl group, and a terphenyl group, but embodiments are not limited thereto.

R₅ and R₆ may optionally be bound via a first linking group to form a substituted or unsubstituted C₅-C₃₀ carbocyclic group or a substituted or unsubstituted C₁-C₃₀ heterocyclic group (e.g., a 5 to 7-membered cyclic group having 5 or 6 carbon number; or a 5 to 7-membered cyclic group having 5 or 6 carbon number substituted with at least one selected from deuterium, a cyano group, —F, a C₁-C₁₀ alkyl group, and a C₆-C₁₄ aryl group).

In an embodiment, in Formula 1, T₁ to T₃ may each independently be selected from *—N[(L₅)ₐ₅-(R₅)]—*′, *—B(R₅)—*, *—C(R₅)(R₆)—*′, *—Si(R₅)(R₆)—*′, *—S—*—O—*′, and *—C(═O)—*′, but embodiments are not limited thereto.

In one or more embodiments, in Formula 1, T₁ to T₃ may each independently be selected from *—C(R₅)(R₆)—*′, *—Si(R₅)(R₆)—*′, and *—Ge(R₅)(R₆)—*,

R₅ and R₆ may be bound via a first linking group,

the first linking group may be selected from a single bond, *—N[(L₉)ₐ₉-(R₉)]—*′, *—B(R₉)—*′, *—P(R₉)—*′, *—C(R₉)(R₁₀)—*′, *—Si(R₉)(R₁₀)—*′, *—Ge(R₉)(R₁₀)—*′, *—S—*′, *—Se—*′, *—O—*′, *—C(═O)—*′, *—S(═O)—*′, *—S(═O)₂—*′, *—C(R₉)═C(R₁₀)—*′, *—C(═S)—*′, and *—C≡C—*′,

L₉ and a9 may each be the same as described herein with reference to L₅ and a5, respectively,

R₉ and R₁₀ may each be the same as described herein with reference to R₅, and

* and *′ may each independently indicate a binding site to an adjacent atom, but embodiments are not limited thereto.

In Formula 1, b1 to b3 each indicate the number of groups T₁ to T₃, respectively, and b1 to b3 may each independently be selected from 0, 1, 2, and 3; provided that when b1 is 0, *-(T₁)_{b1}-*′ may be a single bond; when b2 is 0, *-(T₂)_{b2}-*′ may be a single bond; and when b3 is 0, *-(T₃)_{b3}-*′ may be a single bond. When b1 is 2 or greater, at least two T₁ groups may be identical to or different from each other; when b2 is 2 or greater, at least two T₂ groups may be identical to or different from each other; and when b3 is 2 or greater, at least two T₃ groups may be identical to or different from each other.

In an embodiment, in Formula 1, b1 to b3 may each independently be 0 or 1.

In one or more embodiments, in Formula 1, b1 to b3 may each independently be 0 or 1, and a sum of b1, b2, and b3 may be 1.

In one or more embodiments, in Formula 1, b1 may be 1, and b2 and b3 may each be 0 or 1.

In one or more embodiments, in Formula 1, b1 may be 1, and b2 and b3 may each be 0, but embodiments are not limited thereto.

R₁ to R₆ may each independently be selected from hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉), wherein Q₁ to Q₉ may be the same as those described herein.

In some embodiments, R₁ to R₆ may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;

a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and

—N(Q₁)(Q₂), —Si(Q₃)(Q₄)(Q₅), —B(Q₆)(Q₇), and —P(═O)(Q₈)(Q₉),

wherein Q₁ to Q₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

In an embodiment, R₁ to R₆ may each independently be selected from:

hydrogen, deuterium, —F, a cyano group, a nitro group, —SF₅, a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group;

a methyl group, an ethyl group, an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, a tert-decyl group, a methoxy group, an ethoxy group, a propoxy group, a butoxy group, a pentoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group, each substituted with at least one selected from deuterium, —F, —CD₃, —CD₂H, —CDH₂, —CF₃, —CF₂H, —CFH₂, a cyano group, a nitro group, a C₁-C₁₀ alkyl group, a C₁-C₁₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, a carbazolyl group, a dibenzofuranyl group, and a dibenzothiophenyl group; and

—N(Q₁)(Q₂), —Si(Q₃)(Q₄)(Q₅), —B(Q₆)(Q₇), and —P(═O)(Q₈)(Q₉),

wherein Q₁ to Q₉ may each independently be selected from:

—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and

an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

In one or more embodiments, R₁ to R₆ may each independently be selected from hydrogen, deuterium, —F, a cyano group, a nitro group, —SF₅, —CH₃, —CD₃, —CD₂H, —CDH₂, —CF₃, —CF₂H, —CFH₂, groups represented by Formulae 9-1 to 9-20, groups represented by Formulae 10-1 to 10-152, and —Si(Q₃)(Q₄)(Q₅) (wherein Q₃ to Q₅ may be the same as those described herein), but embodiments are not limited thereto:

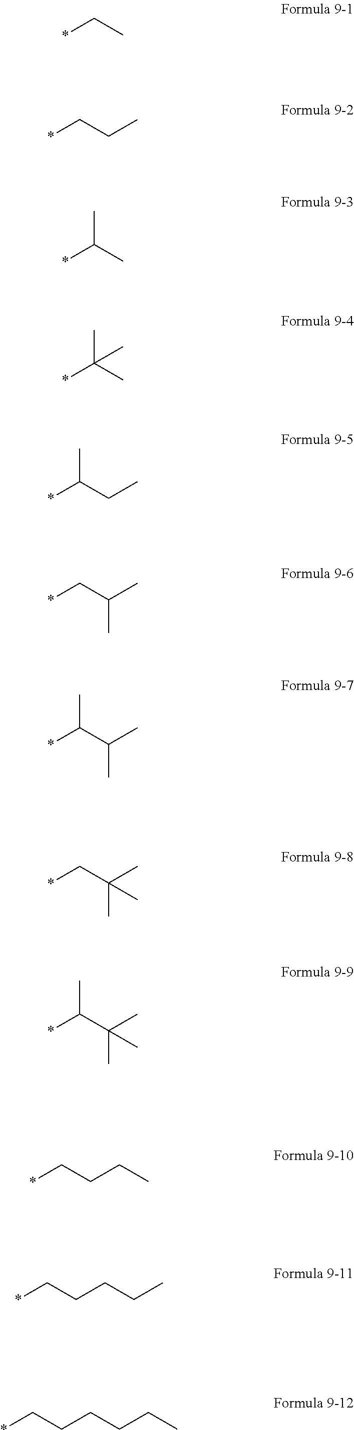

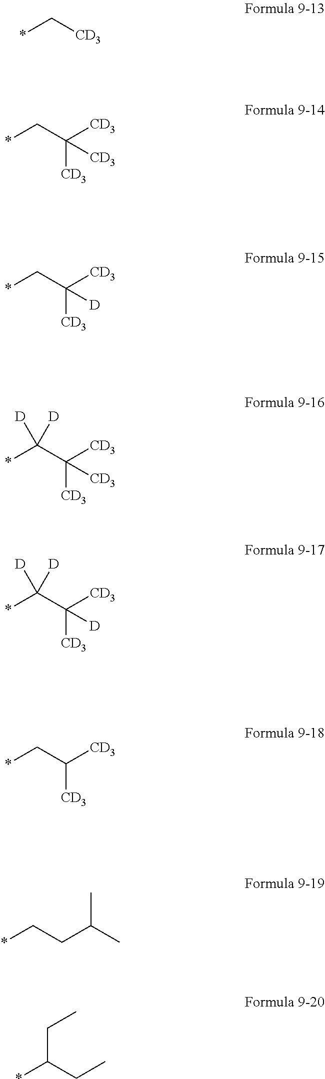

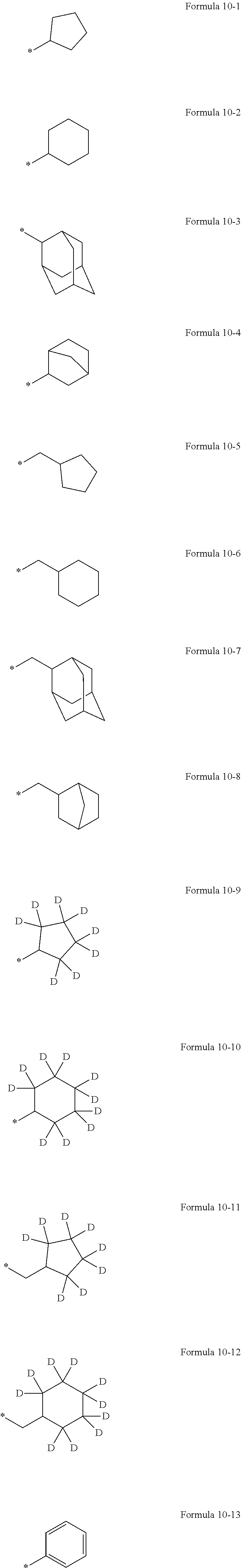

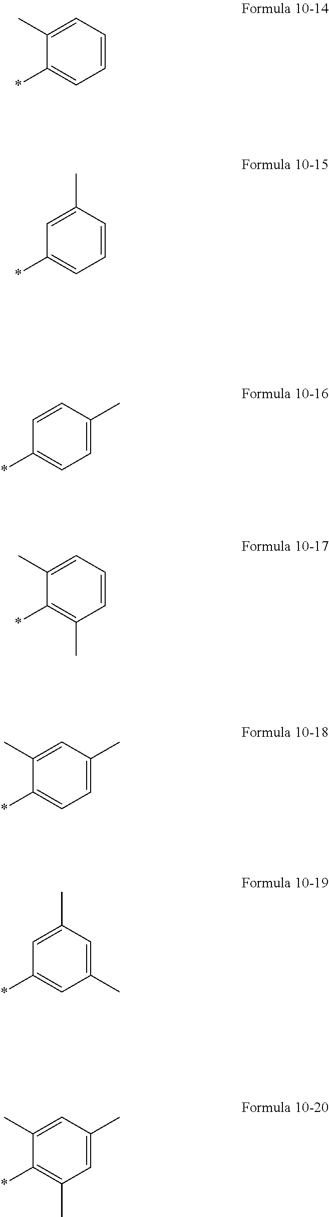

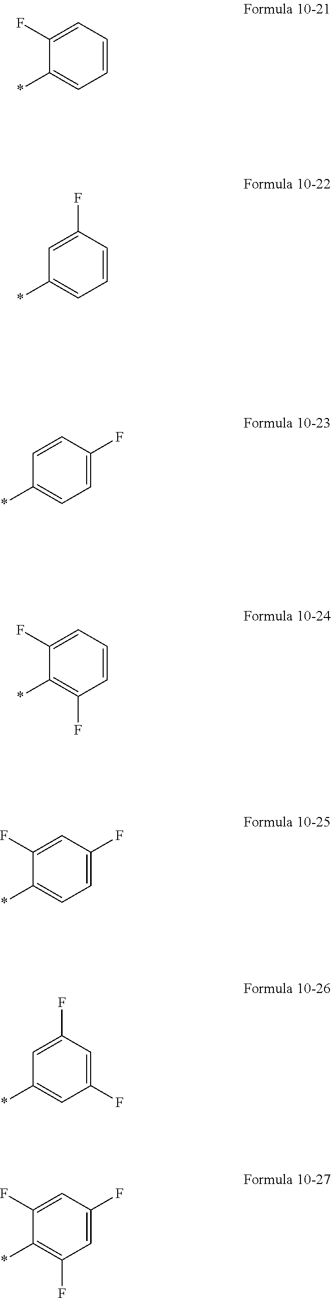

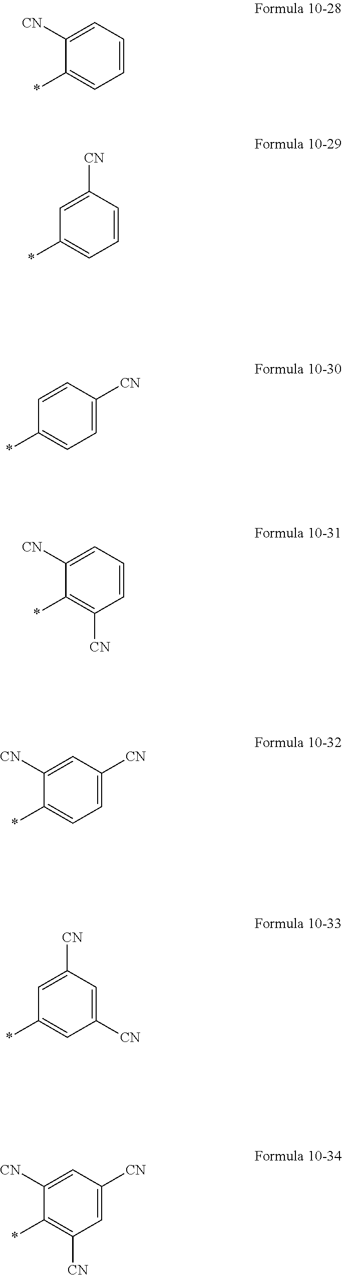

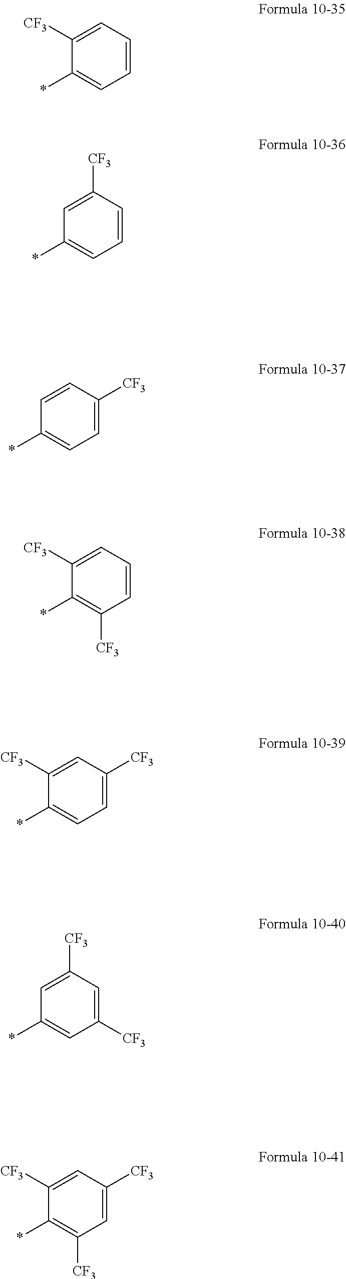

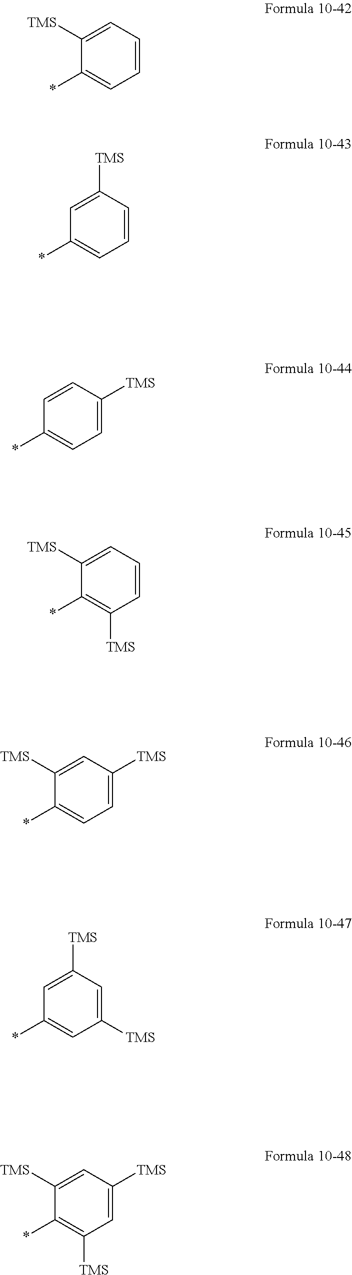

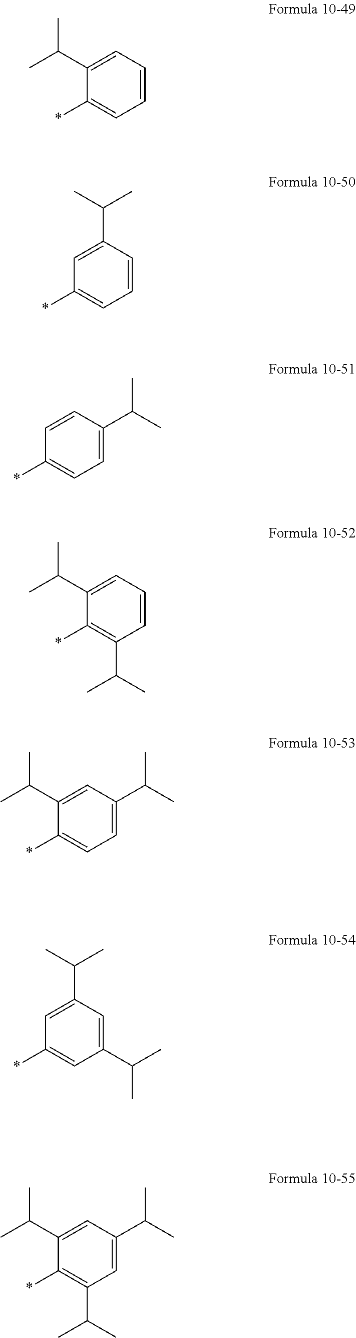

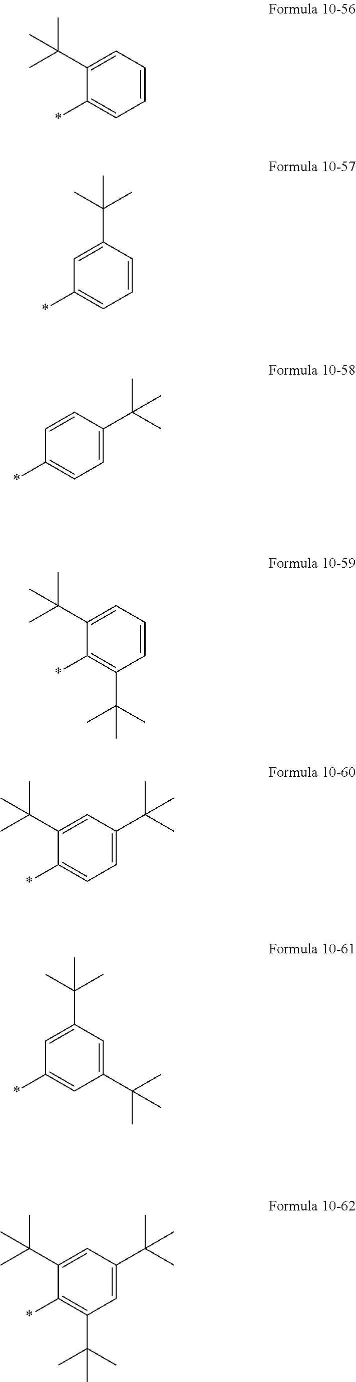

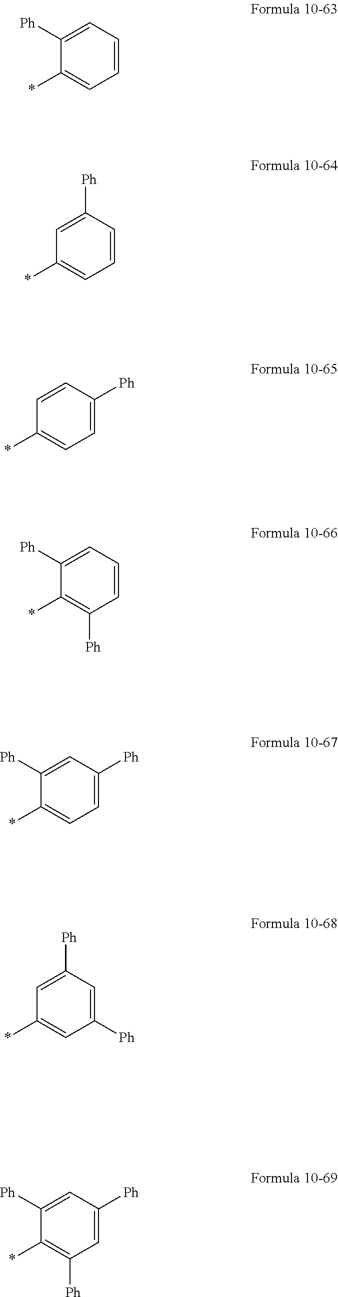

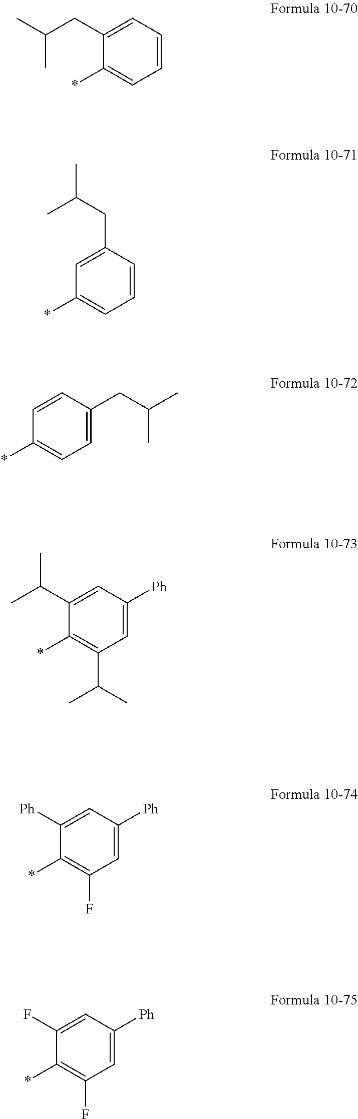

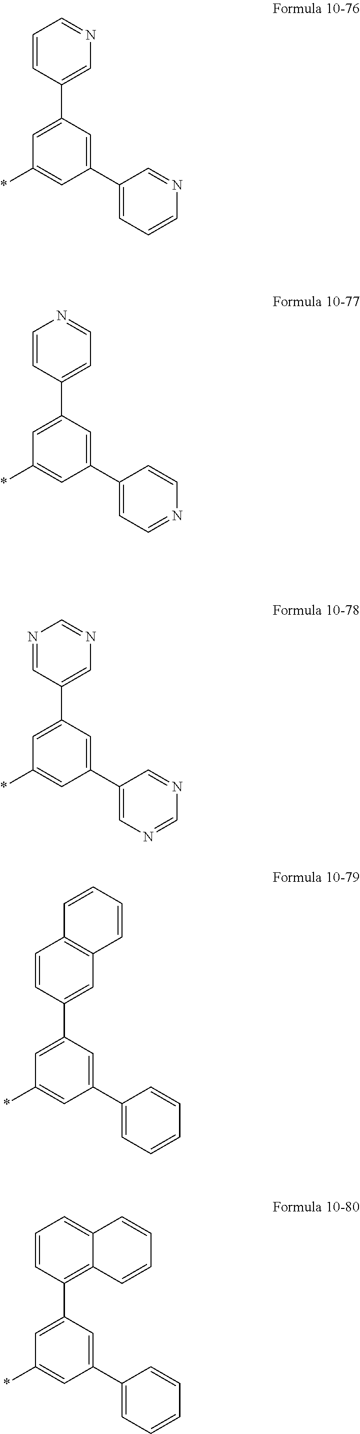 [SMILES: *c1cc(-c2cccnc2)cc(-c2cccnc2)c1 ; *c1cc(-c2ccncc2)cc(-c2ccncc2)c1 ; *c1cc(-c2cncnc2)cc(-c2cncnc2)c1 ; *c1cc(-c2ccccc2)cc(-c2ccc3ccccc3c2)c1 ; *c1cc(-c2ccccc2)cc(-c2cccc3ccccc23)c1]

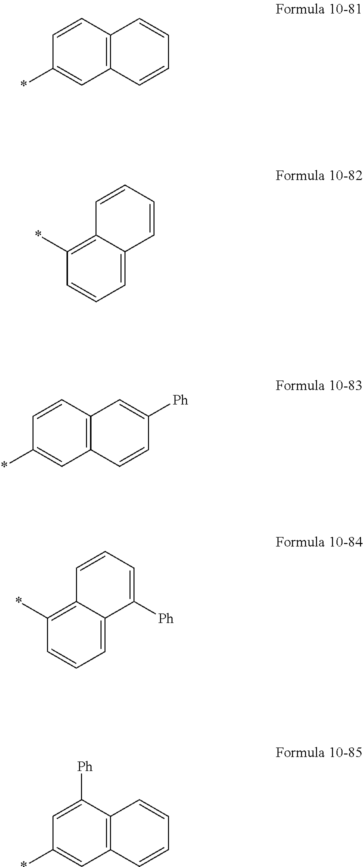 [SMILES: *c1ccc2ccccc2c1 ; *c1cccc2ccccc12 ; *c1ccc2cc(-c3ccccc3)ccc2c1 ; *c1cccc2c(-c3ccccc3)cccc12 ; *c1cc(-c2ccccc2)c2ccccc2c1]

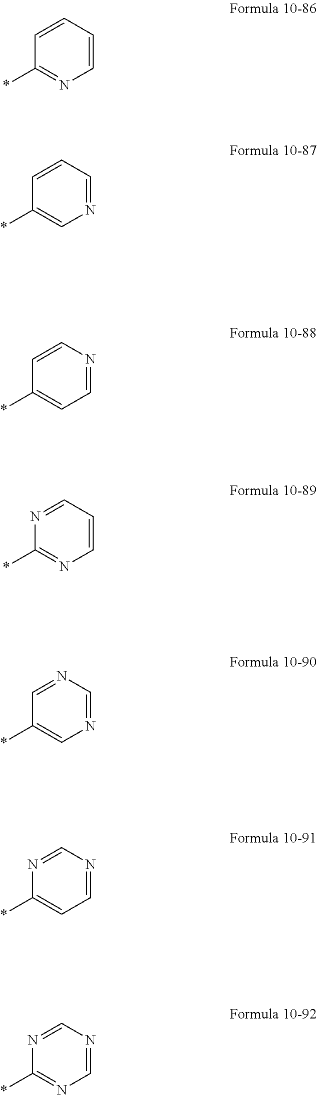

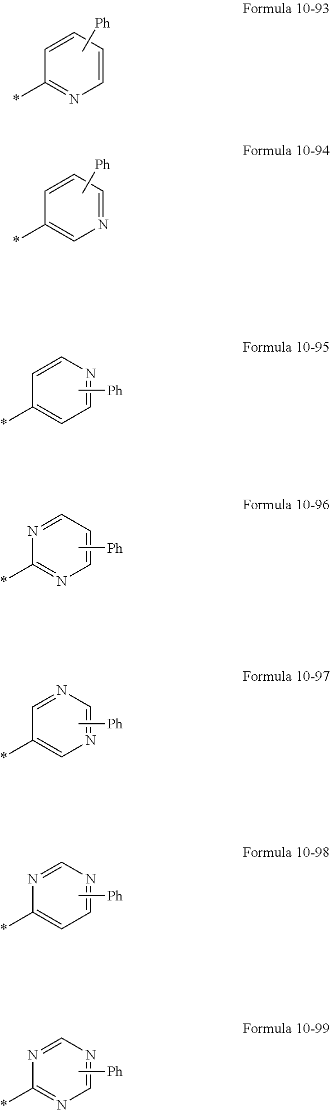

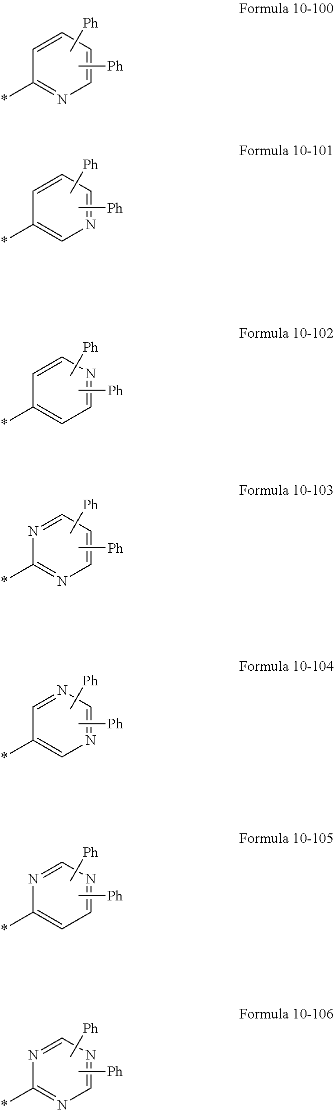

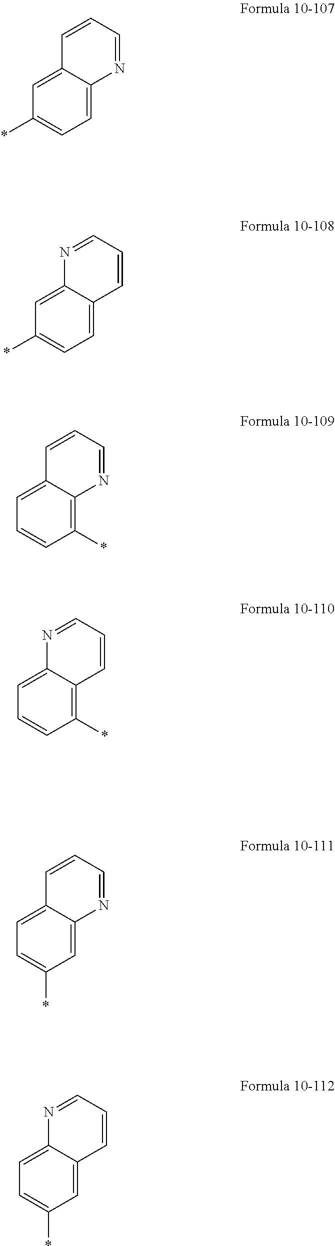 [SMILES: *c1ccc2ncccc2c1 ; *c1ccc2cccnc2c1 ; *c1cccc2cccnc12 ; *c1cccc2ncccc12 ; *c1ccc2cccnc2c1 ; *c1ccc2ncccc2c1]

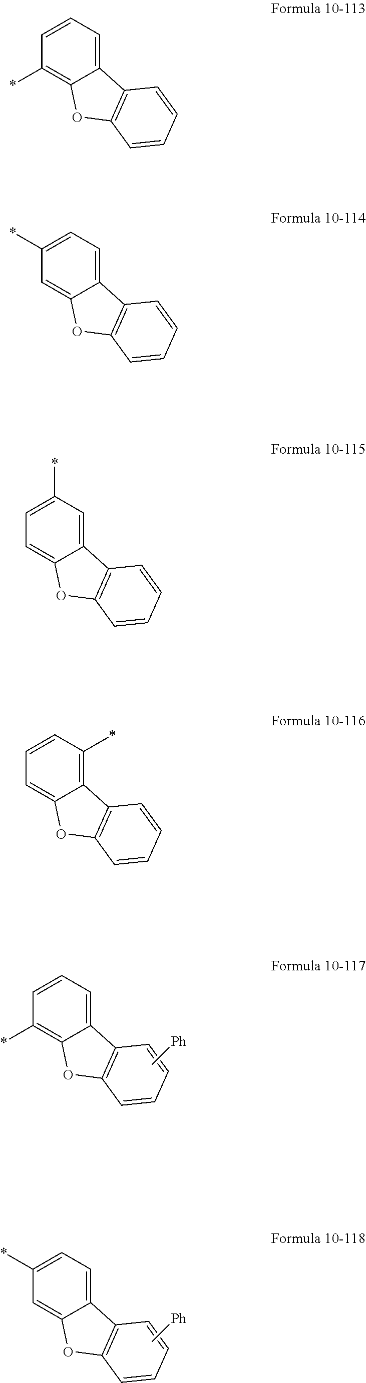

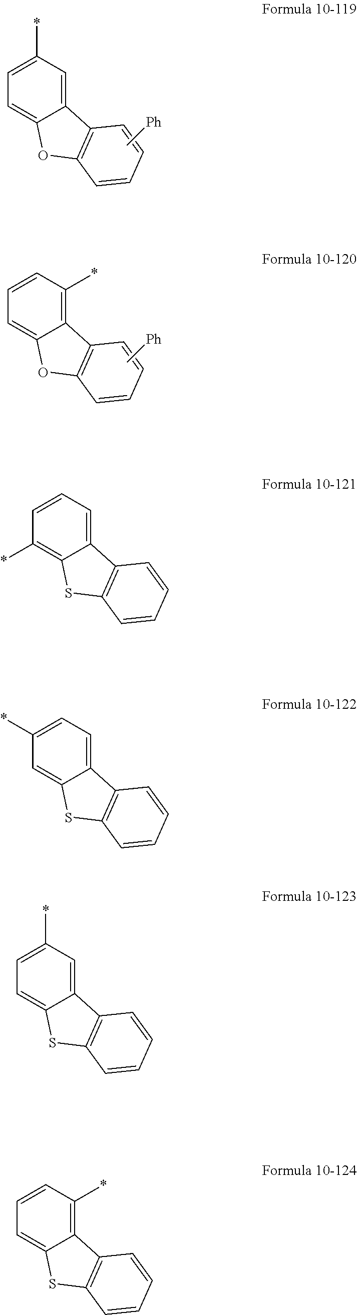

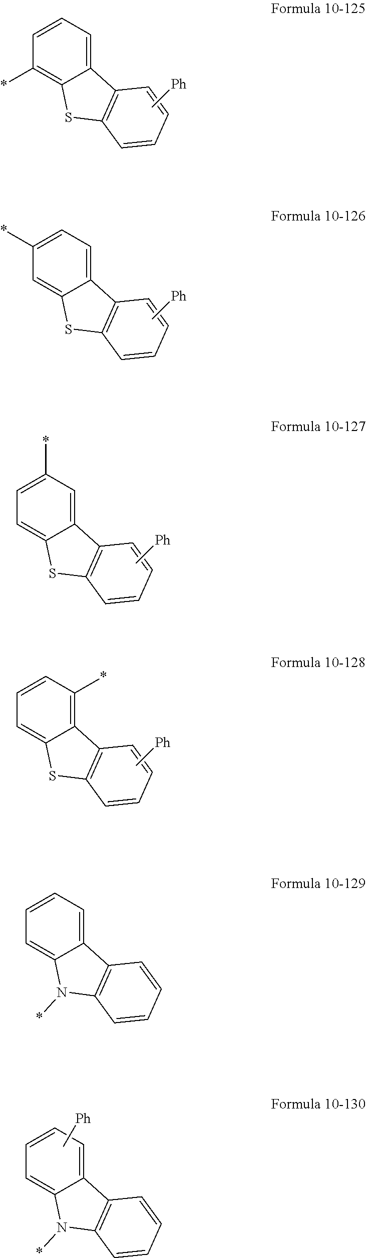

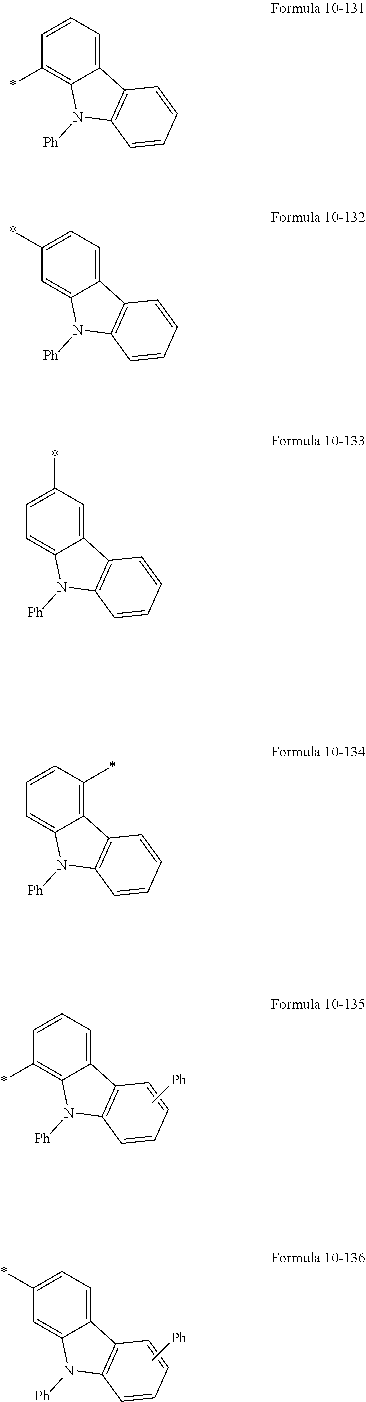

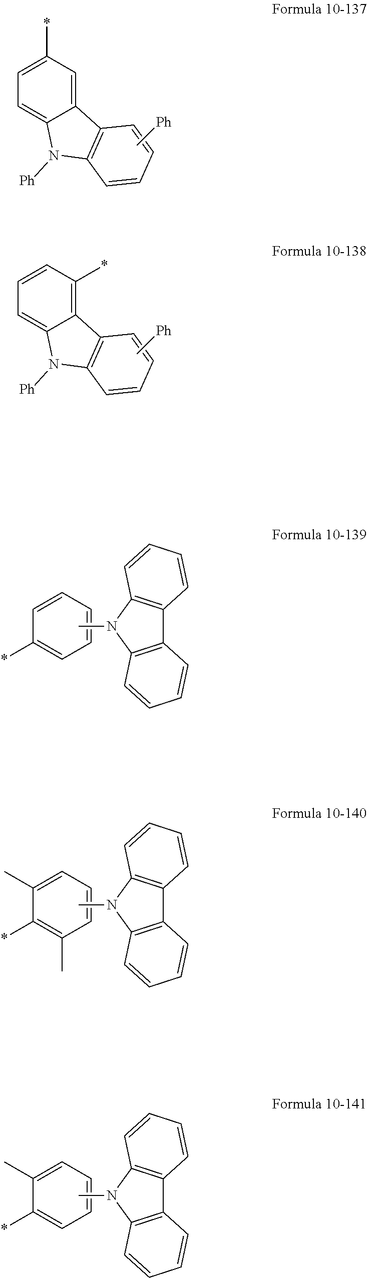

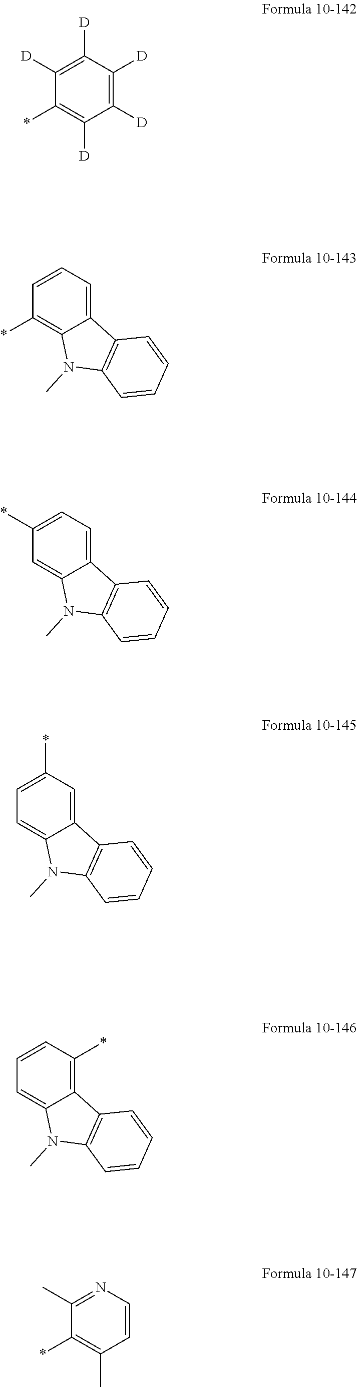

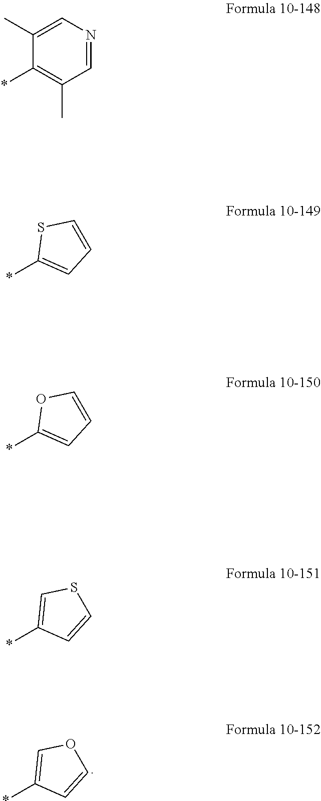

In Formulae 9-1 to 9-20 and 10-1 to 10-152, * indicates a binding site to an adjacent atom, “Ph” represents a phenyl group, and “TMS” represents a trimethylsilyl group.

In Formula 1, a1 to a4 respectively indicate numbers of groups R₁ to R₄, and a1 to a4 may each independently be 0, 1, 2, 3, 4, or 5. When a1 is 2 or greater, at least two R₁ groups may be identical to or different from each other, when a2 is 2 or greater, at least two R₂ groups may be identical to or different from each other, when a3 is 2 or greater, at least two R₃ groups may be identical to or different from each other, when a4 is 2 or greater, at least two R₄ groups may be identical to or different from each other, but embodiments are not limited thereto.

In Formula 1, two of R₁ groups in the number of a1 may optionally be bound to form a substituted or unsubstituted C₅-C₃₀ carbocyclic group or a substituted or unsubstituted C₁-C₃₀ heterocyclic group, two of R₂ groups in the number of a2 may optionally be bound to form a substituted or unsubstituted C₅-C₃₀ carbocyclic group or a substituted or unsubstituted C₁-C₃₀ heterocyclic group, two of R₃ groups in the number of a3 may optionally be bound to form a substituted or unsubstituted C₅-C₃₀ carbocyclic group or a substituted or unsubstituted C₁-C₃₀ heterocyclic group, two of R₄ groups in the number of a4 may optionally be bound to form a substituted or unsubstituted C₅-C₃₀ carbocyclic group or a substituted or unsubstituted C₁-C₃₀ heterocyclic group, at least two of R₁ to R₄, wherein the at least two of R₁ to R₄ may be adjacent to each other, may optionally be bound to form a substituted or unsubstituted C₅-C₃₀ carbocyclic group or a substituted or unsubstituted C₁-C₃₀ heterocyclic group, and at least one of R₅ and R₆ and at least one of R₁ to R₄ may optionally be bound to form a substituted or unsubstituted C₅-C₃₀ carbocyclic group or a substituted or unsubstituted C₁-C₃₀ heterocyclic group.

In some embodiments, in Formula 1, i) a substituted or unsubstituted C₅-C₃₀ carbocyclic group or a substituted or unsubstituted C₁-C₃₀ heterocyclic group formed by an optional bond between two R₁ groups in the number of a1, ii) a substituted or unsubstituted C₅-C₃₀ carbocyclic group or a substituted or unsubstituted C₁-C₃₀ heterocyclic group formed by an optional bond between two R₂ groups in the number of a2, iii) a substituted or unsubstituted C₅-C₃₀ carbocyclic group or a substituted or unsubstituted C₁-C₃₀ heterocyclic group formed by an optional bond between two R₃ groups in the number of a3, iv) a substituted or unsubstituted C₅-C₃₀ carbocyclic group or a substituted or unsubstituted C₁-C₃₀ heterocyclic group formed by an optional bond between two R₄ groups in the number of a4, v) a substituted or unsubstituted C₅-C₃₀ carbocyclic group or a substituted or unsubstituted C₁-C₃₀ heterocyclic group formed by an optional bond between at least two of R₁ to R₄, wherein the at least two of R₁ to R₄ may be adjacent to each other, and vi) a substituted or unsubstituted C₅-C₃₀ carbocyclic group or a substituted or unsubstituted C₁-C₃₀ heterocyclic group formed by an optional bond between at least one of R₅ and R₆ and at least one of R₁ to R₄, may each independently be selected from:

a pentadiene group, a cyclohexane group, a cycloheptane group, an adamantane group, a bicyclo-heptane group, a bicyclo-octane group, a benzene group, a pyridine group, a pyrimidine group, a pyrazine group, a pyridazine group, a naphthalene group, an anthracene group, a tetracene group, a phenanthrene group, a dihydronaphthalene group, a phenalene group, a benzothiophene group, a benzofuran group, an indene group, an indole group, a benzosilole group, an azabenzothiophene group, an azabenzofuran group, an azaindene group, an azaindole group, and an azabenzosilole group; and

a pentadiene group, a cyclohexane group, a cycloheptane group, an adamantane group, a bicyclo-heptane group, a bicyclo-octane group, a benzene group, a pyridine group, a pyrimidine group, a pyrazine group, a pyridazine group, a naphthalene group, an anthracene group, a tetracene group, a phenanthrene group, a dihydronaphthalene group, a phenalene group, a benzothiophene group, a benzofuran group, an indene group, an indole group, a benzosilole group, an azabenzothiophene group, an azabenzofuran group, an azaindene group, an azaindole group, and an azabenzosilole group, each substituted with at least one R₁ₐ, but embodiments are not limited thereto.

R₁ₐ may be the same as described herein with reference to R₁.

In an embodiment, a moiety represented by

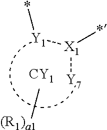

in Formula 1 may be selected from groups represented by Formulae CY1-1 to CY1-6:

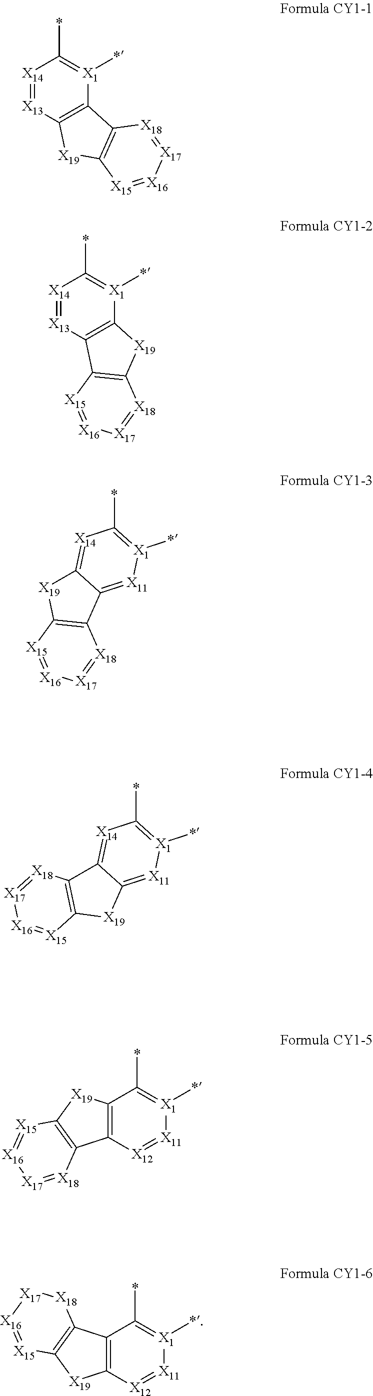

wherein, in Formulae CY1-1 to CY1-6,

X₁ may be N,

X₁₁ may be N or C(R₁₁), X₁₂ may be N or C(R₁₂), X₁₃ may be N or C(R₁₃), X₁₄ may be N or C(R₁₄), X₁₅ may be N or C(R₁₅), X₁₆ may be N or C(R₁₆), X₁₇ may be N or C(R₁₇), X₁₈ may be N or C(R₁₈),

X₁₉ may be selected from *—N[(L₁₉)ₐ₁₉-(R₁₉)]—*′, *—B(R₁₉)—*′, *—P(R₁₉)—*, *—C(R₁₉ₐ)(R_{19b})—*′, *—Si(R₁₉ₐ)(R_{19b})—*′, *—Ge(R₁₉ₐ)(R_{19b})—*′, *—S—*′, *—Se—*′, *—O—*′, *—C(═O)—*′, *—S(═O)—*′, *—S(═O)₂—*′, and *—C(═S)—*′,

L₁₉ and a19 may each be the same as described herein with reference to L₅ and a5, respectively,

R₁₁ to R₁₉, R₁₉ₐ, and R_{19b} may each independently be the same as described herein with reference to R₁, and

* and *′ may each indicate a binding site to an adjacent atom.

In some embodiments, in Formulae CY1-1 to CY1-6, none, one, or two of X₁₁ to X₁₈ may be N.

In some embodiments, in Formulae CY1-1 to CY1-6, X₁₁ to X₁₄ may each not be N, and none or one of X₁₅ to X₁₈ may be N.

In some embodiments, in Formulae CY1-1 to CY1-6, X₁₁ may be C(R₁₁), X₁₂ may be C(R₁₂), X₁₃ may be C(R₁₃), X₁₄ may be C(R₁₄), X₁₅ may be C(R₁₅), X₁₆ may be C(R₁₆), X₁₇ may be C(R₁₇), and X₁₈ may be C(R₁₈), but embodiments are not limited thereto.

In some embodiments, in Formulae CY1-1 to CY1-6, X₁₁ may be C(R₁₁), X₁₂ may be C(R₁₂), X₁₃ may be C(R₁₃), X₁₄ may be C(R₁₄), X₁₅ may be N, X₁₆ may be C(R₁₆), X₁₇ may be C(R₁₇), and X₁₈ may be C(R₁₈), but embodiments are not limited thereto.

In one or more embodiments, a moiety represented by

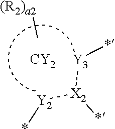

in Formula 1 may be selected from groups represented by Formulae CY2-1 to CY2-21:

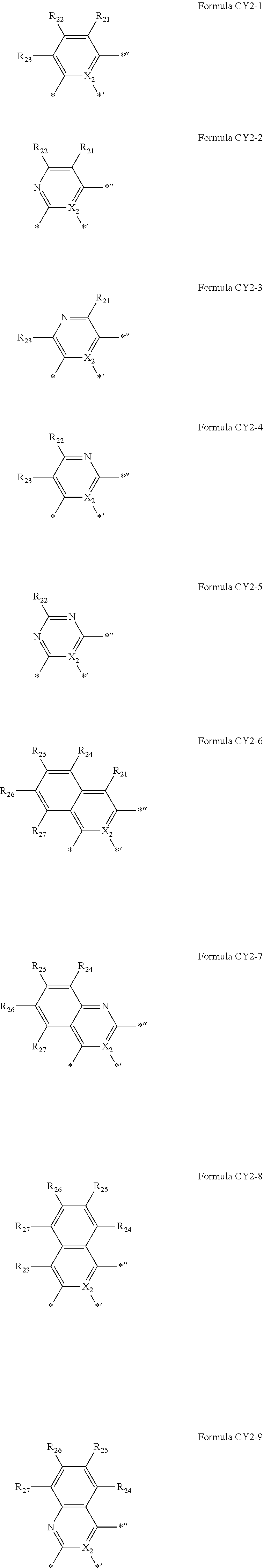

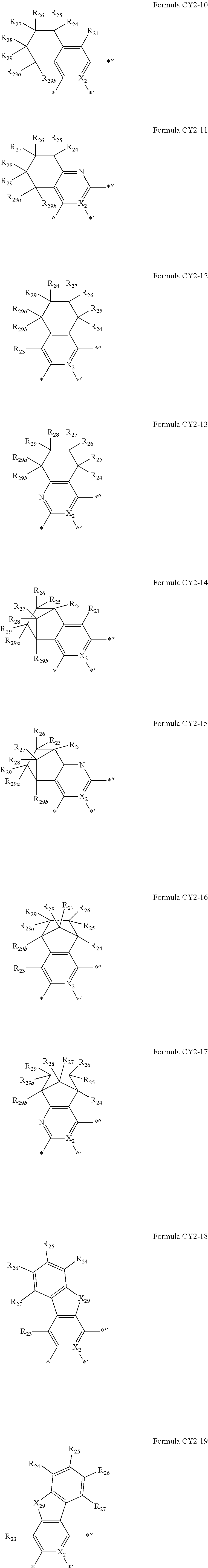

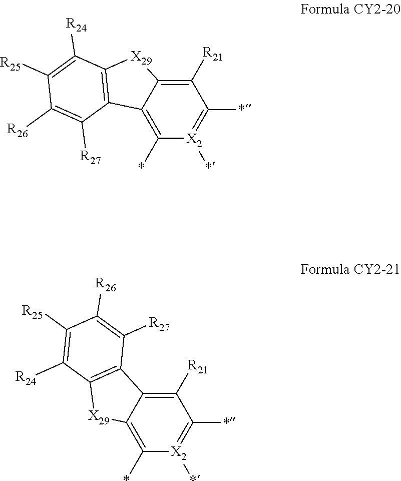

wherein, in Formulae CY2-1 to CY2-21,

X₂ may be selected from C and N,

X₂₉ may be selected from *—N[(L₂₉)ₐ₂₉-(R₂₉)]—*′, *—B(R₂₉)—*′, *—P(R₂₉)—*, *—C(R₂₉ₐ)(R_{29b})—*′, *—Si(R₂₉ₐ)(R_{29b})—*′, *—Ge(R₂₉ₐ)(R_{29b})—*′, *—S—*′, *—Se—*′, *—O—*′, *—C(═O)—*′, *—S(═O)—*′, *—S(═O)₂—*′, and *—C(═S)—*′,

L₂₉ and a29 may each be the same as described herein with reference to L₅ and a5, respectively,

R₂₁ to R₂₉, R₂₉ₐ, and R_{29b} may each independently be the same as described herein with reference to R₂, and

*, *′, and *″ may each indicate a binding site to an adjacent atom.

In one or more embodiments, a moiety represented by

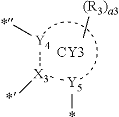

in Formula 1 may be selected from groups represented by Formulae CY3-1 to CY3-21:

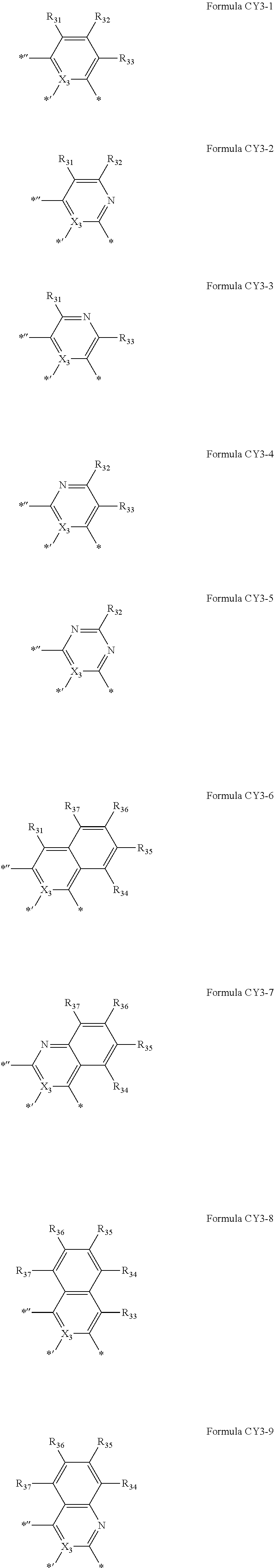

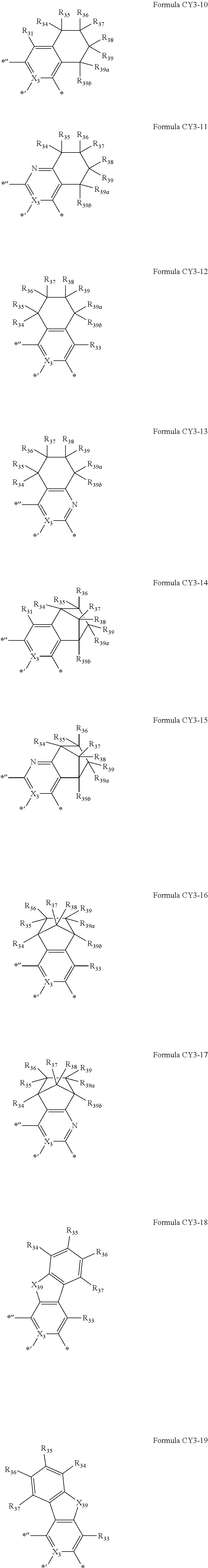

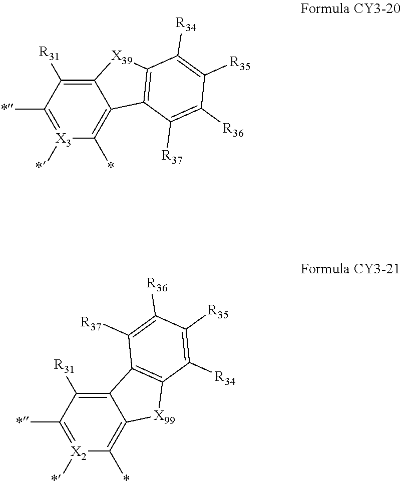

wherein, in Formulae CY3-1 to CY3-21,

X₃ may be selected from C and N,

X₃₉ may be selected from *—N[(L₃₉)ₐ₃₉-(R₃₉)]—*′, *—B(R₃₉)—*′, *—P(R₃₉)—*, *—C(R₃₉ₐ)(R_{39b})—*′, *—Si(R₃₉ₐ)(R_{39b})—*′, *—Ge(R₃₉ₐ)(R_{39b})—*′, *—S—*′, *—Se—*′, *—O—*′, *—C(═O)—*′, *—S(═O)—*′, *—S(═O)₂—*′, and *—C(═S)—*′,

L₃₉ and a39 may each be the same as described herein with reference to L₅ and a5, respectively,

R₃₁ to R₃₉, R₃₉ₐ, and R_{39b} may each independently be the same as described herein with reference to R₃, and

*, *′, and *″ may each indicate a binding site to an adjacent atom.

In one or more embodiments, a moiety represented by

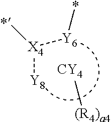

in Formula 1 may be selected from groups represented by Formulae CY4-1 to CY4-38:

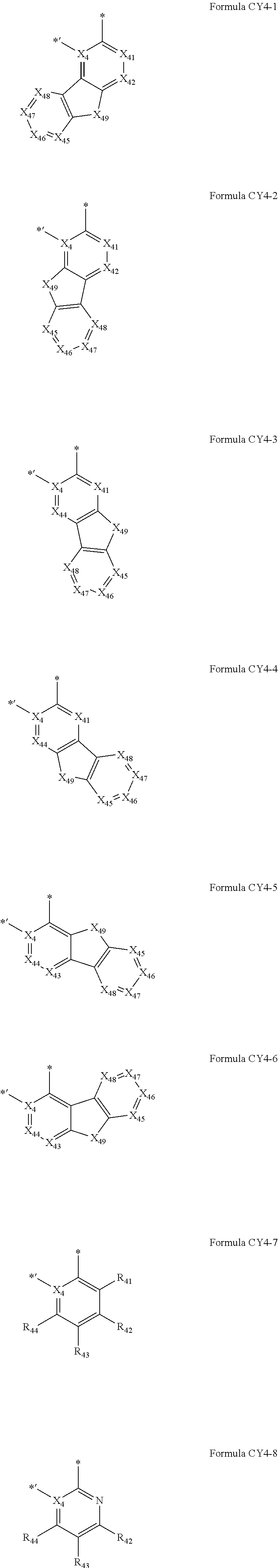

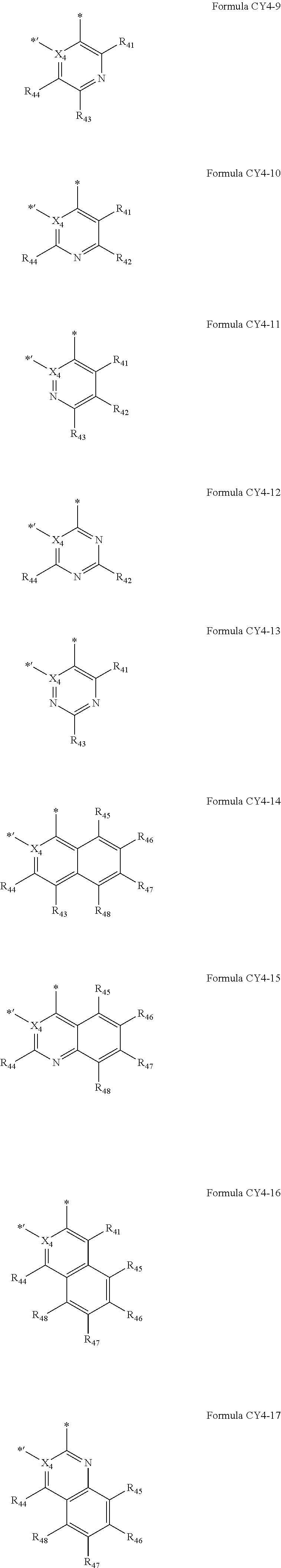

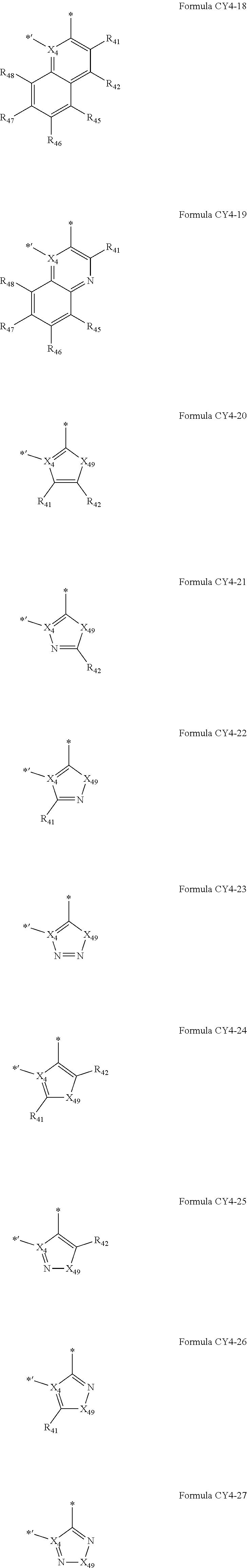

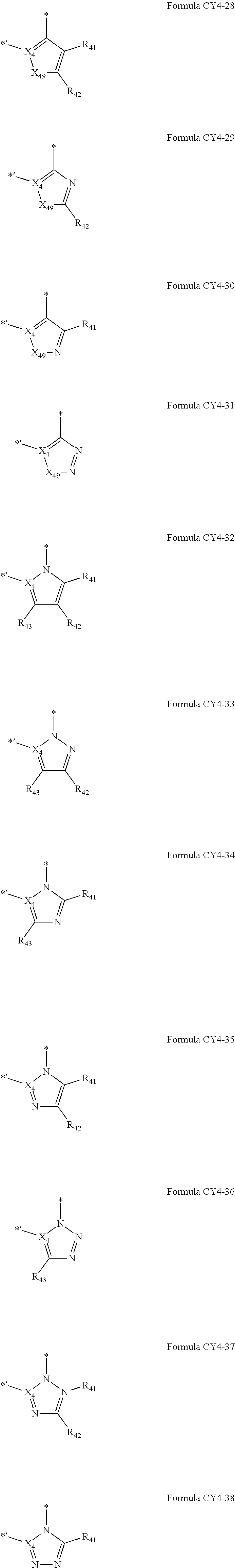

wherein, in Formulae CY4-1 to CY4-38,

X₄ may be selected from C and N,

X₄₁ may be N or C(R₄₁), X₄₂ may be N or C(R₄₂), X₄₃ may be N or C(R₄₃), X₄₄ may be N or C(R₄₄), X₄₅ may be N or C(R₄₅), X₄₆ may be N or C(R₄₆), X₄₇ may be N or C(R₄₇), X₄₈ may be N or C(R₄₈),

X₄₉ may be selected from *—N[(L₄₉)ₐ₄₉-(R₄₉)]—*′, *—B(R₄₉)—*′, *—P(R₄₉)—*, *—C(R₄₉ₐ)(R_{49b})—*′, *—Si(R₄₉ₐ)(R_{49b})—*′, *—Ge(R₄₉ₐ)(R_{49b})—*′, *—S—*′, —Se—*′, *—O—*′, *—C(═O)—*′, *—S(═O)—*′, *—S(═O)₂—*′, and *—C(═S)—*′,

L₄₉ and a49 may each be the same as described herein with reference to L₅ and a5, respectively,

R₄₁ to R₄₉, R₄₉ₐ, and R_{49b} may each independently be the same as described herein with reference to R₄, and

* and *′ may each indicate a binding site to an adjacent atom.

In some embodiments, in Formulae CY4-1 to CY4-6, none, one, or two of X₄₁ to X₄₈ may be N.

In some embodiments, in Formulae CY4-1 to CY4-6, X₄₁ to X₄₄ may each not be N, and none or one of X₄₅ to X₄₈ may be N.

In some embodiments, in Formulae CY4-1 to CY4-6, X₄₁ may be C(R₄₁), X₄₂ may be C(R₄₂), X₄₃ may be C(R₄₃), X₄₄ may be C(R₄₄), X₄₅ may be C(R₄₅), X₄₆ may be C(R₄₆), X₄₇ may be C(R₄₇), and X₄₈ may be C(R₄₈), but embodiments are not limited thereto.

In some embodiments, in Formulae CY4-1 to CY4-6, X₄₁ may be C(R₄₁), X₄₂ may be C(R₄₂), X₄₃ may be C(R₄₃), X₄₄ may be C(R₄₄), X₄₅ may be N, X₄₆ may be C(R₄₆), X₄₇ may be C(R₄₇), and X₄₈ may be C(R₄₈), but embodiments are not limited thereto.

Two of R₁₁ to R₁₈ may optionally be bound to form a substituted or unsubstituted C₅-C₃₀ carbocyclic group or a substituted or unsubstituted C₁-C₃₀ heterocyclic group, two of R₂₁ to R₂₈ may optionally be bound to form a substituted or unsubstituted C₅-C₃₀ carbocyclic group or a substituted or unsubstituted C₁-C₃₀ heterocyclic group, two of R₃₁ to R₃₈ may optionally be bound to form a substituted or unsubstituted C₅-C₃₀ carbocyclic group or a substituted or unsubstituted C₁-C₃₀ heterocyclic group, and two of R₄₁ to R₄₈ may optionally be bound to form a substituted or unsubstituted C₅-C₃₀ carbocyclic group or a substituted or unsubstituted C₁-C₃₀ heterocyclic group.

In some embodiments, i) a substituted or unsubstituted C₅-C₃₀ carbocyclic group or a substituted or unsubstituted C₁-C₃₀ heterocyclic group formed by an optional bond between two of R₁₁ to R₁₈, ii) a substituted or unsubstituted C₅-C₃₀ carbocyclic group or a substituted or unsubstituted C₁-C₃₀ heterocyclic group formed by an optional bond between two of R₂₁ to R₂₈, iii) a substituted or unsubstituted C₅-C₃₀ carbocyclic group or a substituted or unsubstituted C₁-C₃₀ heterocyclic group formed by an optional bond between two of R₃₁ to R₃₈, and iv) a substituted or unsubstituted C₅-C₃₀ carbocyclic group or a substituted or unsubstituted C₁-C₃₀ heterocyclic group formed by an optional bond between two of R₄₁ to R₄₈ may each independently be selected from:

a pentadiene group, a cyclohexane group, a cycloheptane group, an adamantane group, a bicyclo-heptane group, a bicyclo-octane group, a benzene group, a pyridine group, a pyrimidine group, a pyrazine group, a pyridazine group, a naphthalene group, an anthracene group, a tetracene group, a phenanthrene group, a dihydronaphthalene group, a phenalene group, a benzothiophene group, a benzofuran group, an indene group, an indole group, a benzosilole group, an azabenzothiophene group, an azabenzofuran group, an azaindene group, an azaindole group, and an azabenzosilole group; and

a pentadiene group, a cyclohexane group, a cycloheptane group, an adamantane group, a bicyclo-heptane group, a bicyclo-octane group, a benzene group, a pyridine group, a pyrimidine group, a pyrazine group, a pyridazine group, a naphthalene group, an anthracene group, a tetracene group, a phenanthrene group, a dihydronaphthalene group, a phenalene group, a benzothiophene group, a benzofuran group, an indene group, an indole group, a benzosilole group, an azabenzothiophene group, an azabenzofuran group, an azaindene group, an azaindole group, and an azabenzosilole group, each substituted with at least one R₁ₐ, but embodiments are not limited thereto.

R₁ₐ may be the same as described herein with reference to R₁.

In one or more embodiments, in Formula 1,

a moiety represented by

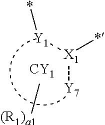

may be a group represented by one of Formulae CY1-1 to CY1-6 (e.g., a group represented by one of Formulae CY1(1) and CY1(2)), and/or

a moiety represented by

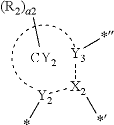

may be a group represented by one of Formulae CY2-1, CY2-6, CY2-8, CY2-10, CY2-12, CY2-14, CY2-16, and CY2-18 to CY2-21 (e.g., a group represented by Formula CY2(1)), and/or

a moiety represented by

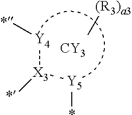

may be a group represented by one of Formulae CY3-1, CY3-6, CY3-8, CY3-10, CY3-12, CY3-14, CY3-16, and CY3-18 to CY3-21 (e.g., a group represented by Formula CY3(1)), and/or

a moiety represented by

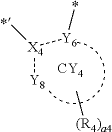

may be a group represented by one of Formulae CY4-1 to CY4-38 (e.g., a group represented by one of Formulae CY4(1) to CY4(7)), but embodiments are not limited thereto:

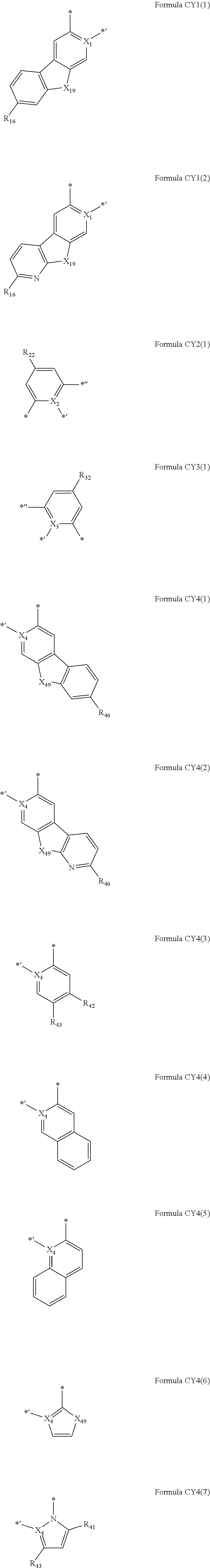

wherein, in Formulae CY1(1), CY1(2), CY2(1), CY3(1), and CY4(1) to CY4(7),

X₁ to X₄, X₁₉, X₄₉, L₁₉, L₄₉, a19, a49, R₁₆, R₁₉, R₁₉ₐ, R_{19b}, R₂₂, R₃₂, R₄₁ to R₄₃, R₄₆, R₄₉, R₄₉ₐ, and R_{49b} may be the same as those describe herein, and *, *′ and *″ may each independently be a binding site to an adjacent atom.

In one or more embodiments, the organometallic compound may be represented by Formula 1(1):

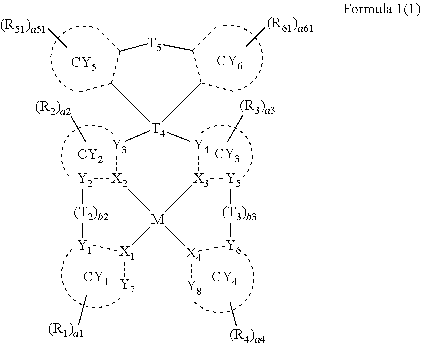

wherein, in Formula 1(1),

M, X₁ to X₄, Y₁ to Y₈, CY₁ to CY₄, T₂, T₃, b2, b3, R₁ to R₄, and a1 to a4 are the same as those described herein,

CY₅ and CY₆ may each independently be a C₅-C₃₀ carbocyclic group or a C₁-C₃₀ heterocyclic group (e.g., a benzene group, a naphthalene group, a pyridine group, or a pyrimidine group),

R₅₁ and R₆₁ may be the same as described herein with reference to R₁,

a51 and a61 may each independently be selected from 0, 1, 2, and 3,

T₄ may be selected from C, silicon (Si), and germanium (Ge),

T₅ may be selected from a single bond, *—N[(L₇)ₐ₇-(R₇)]—*′, *—B(R₇)—*′, *—P(R₇)—*, *—C(R₇)(R₈)—*′, *—Si(R₇)(R₈)—*′, *—Ge(R₇)(R₈)—*′, *—S—*′, *—Se—*′, *—O—*′, *—C(═O)—*′, *—S(═O)—*′, *—S(═O)₂—*′, *—C(R₇)═C(R₈)—*′, *—C(═S)—′, and *—C≡C—*′,

L₇ and a7 may each be the same as described herein with reference to L₅ and a5, respectively,

R₇ and R₈ may each be the same as described herein with reference to R₅, and

* and *′ may each indicate a binding site to an adjacent atom.

In some embodiments, T₅ may be selected from a single bond, *—N[(L₇)ₐ₇-(R₇)]—*′, *—C(R₇)(R₈)—*′, *—Si(R₇)(R₈)—*′, *—S—*, *—O—*′, and *—C(═O)—*′.

The organometallic compound may be selected from Compounds 1 to 54:

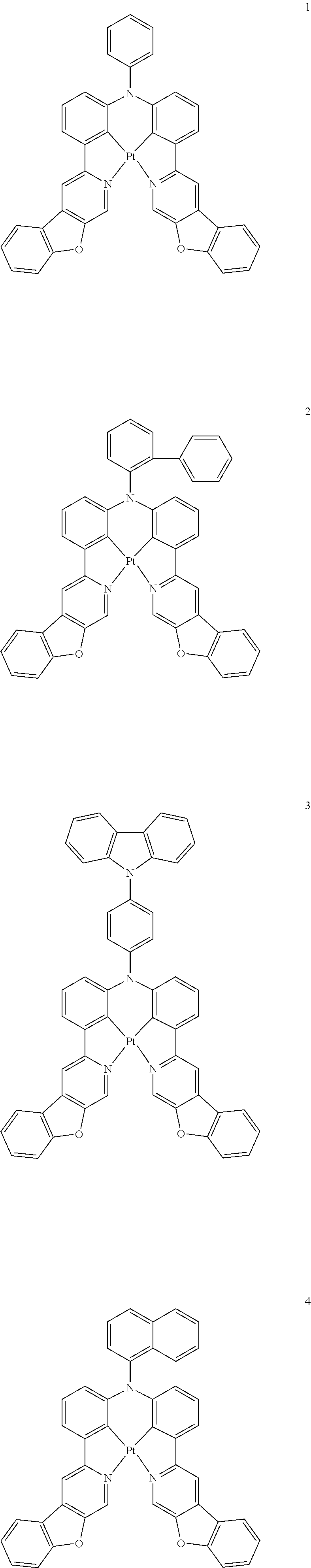

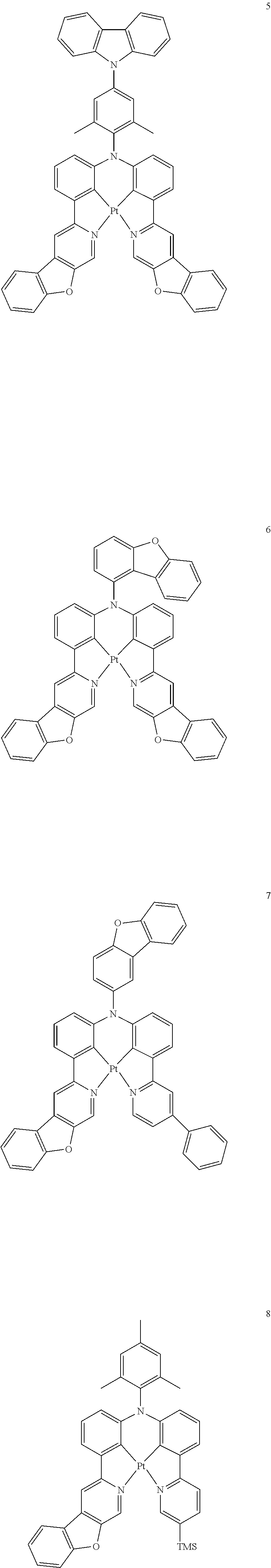

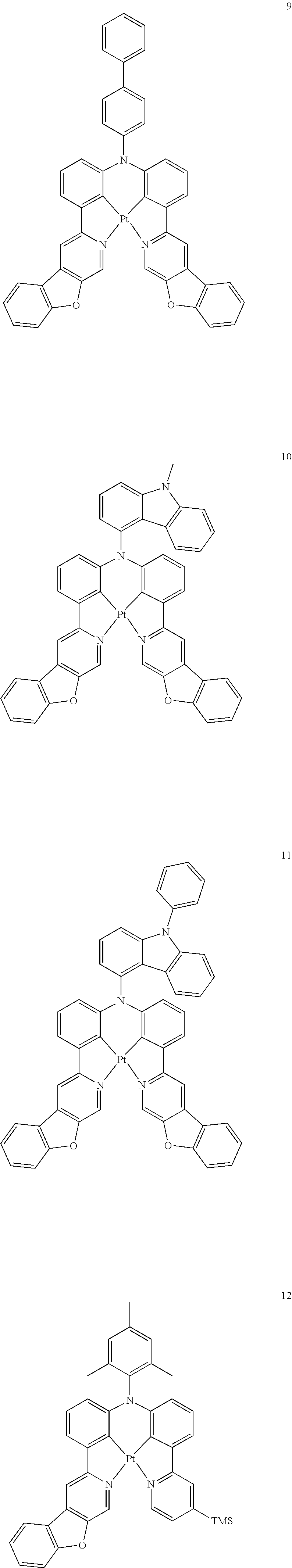

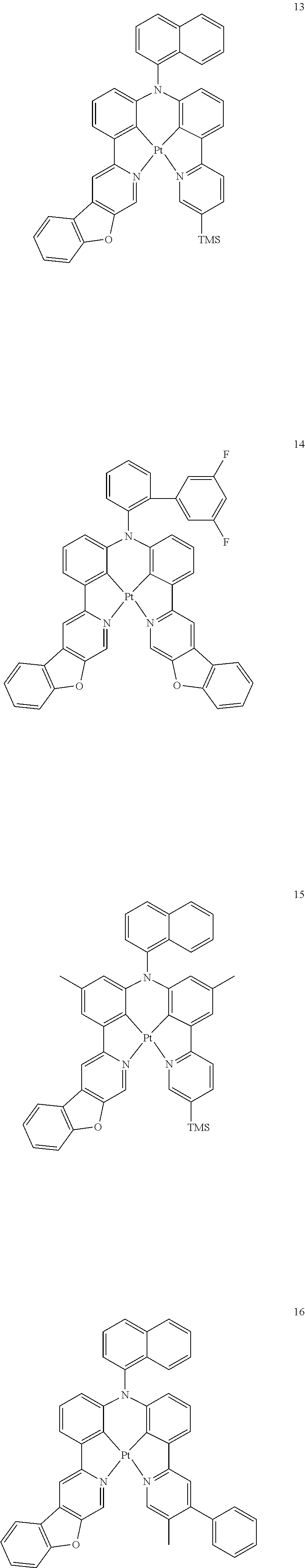

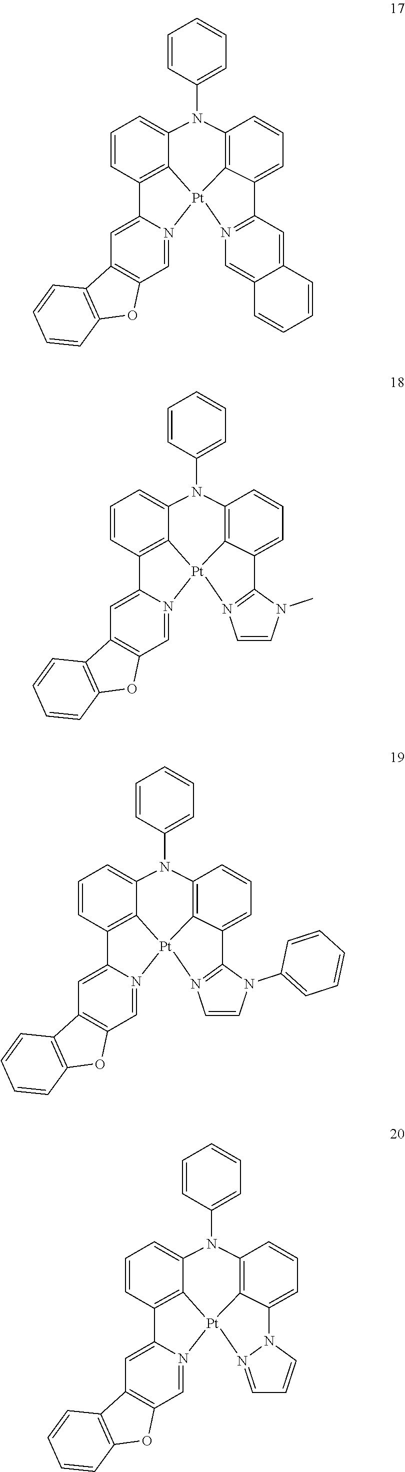

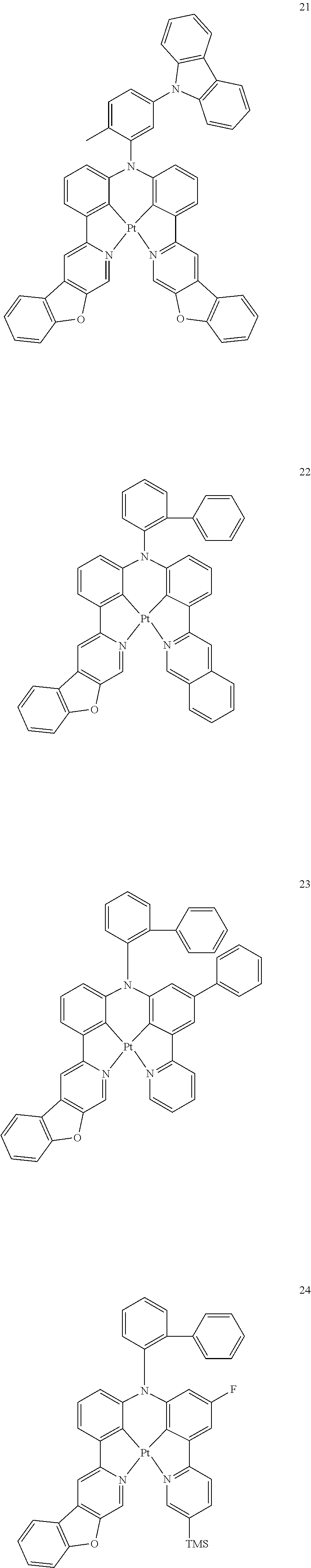

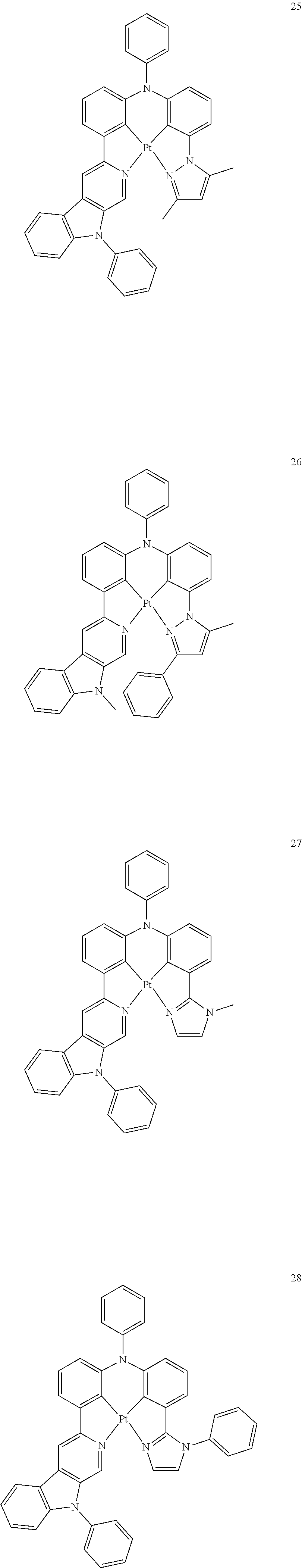

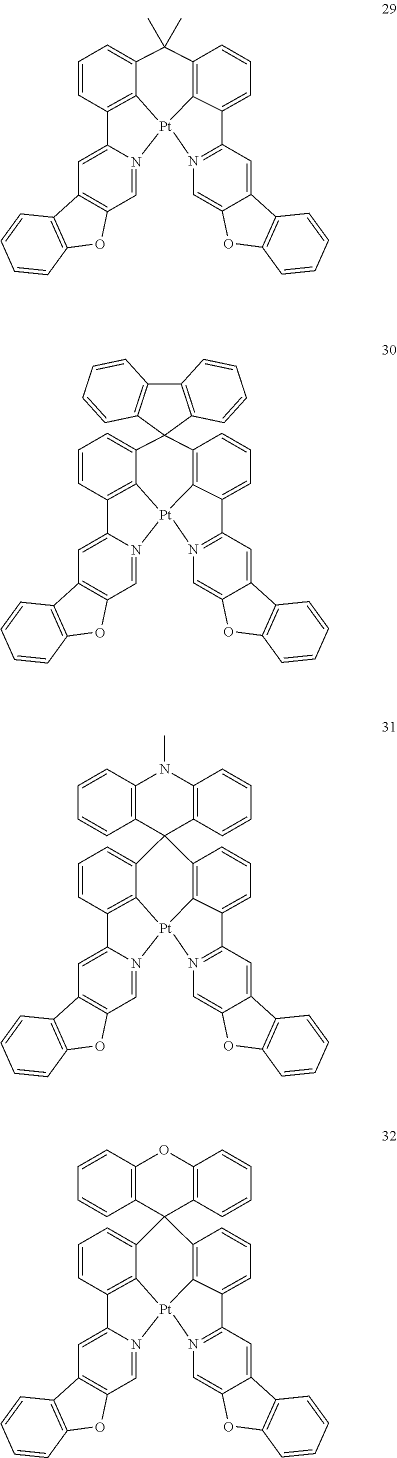

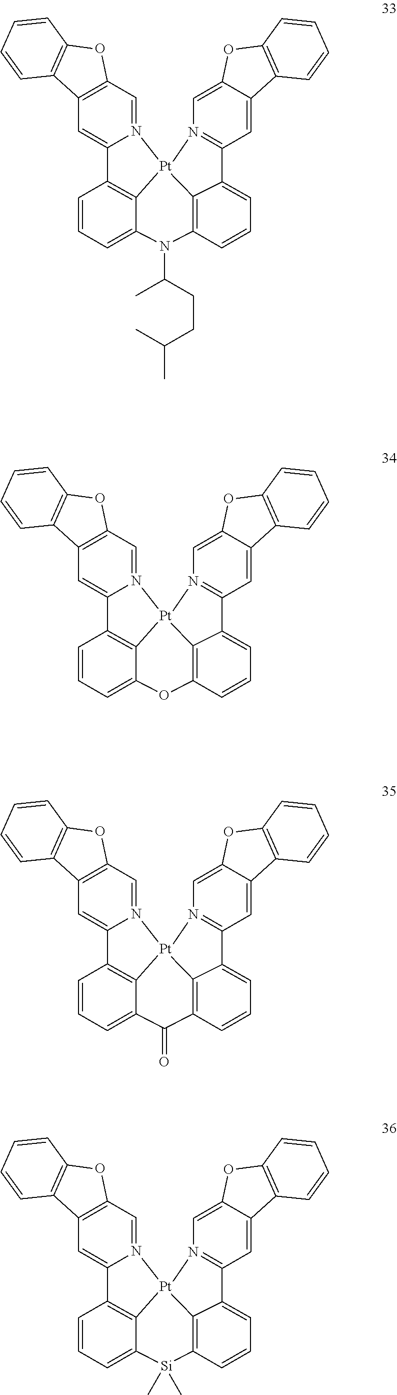

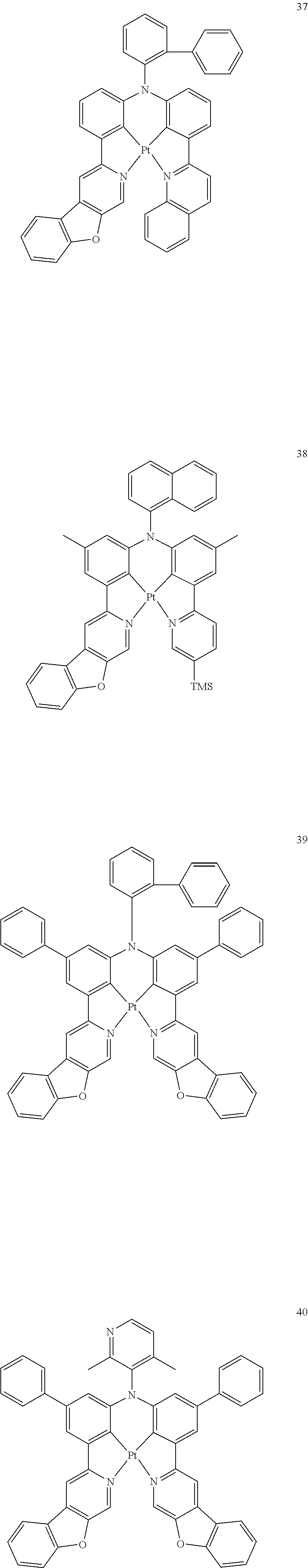

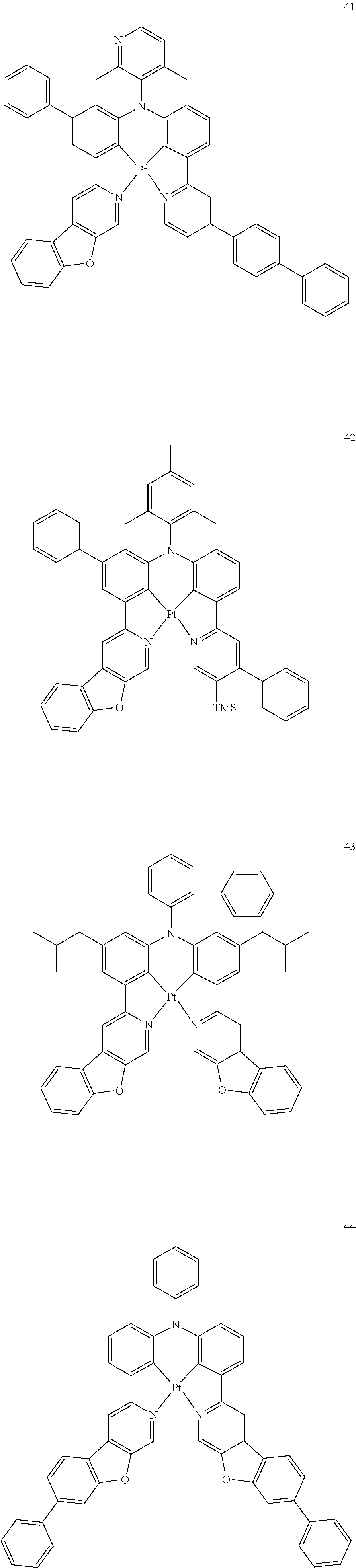

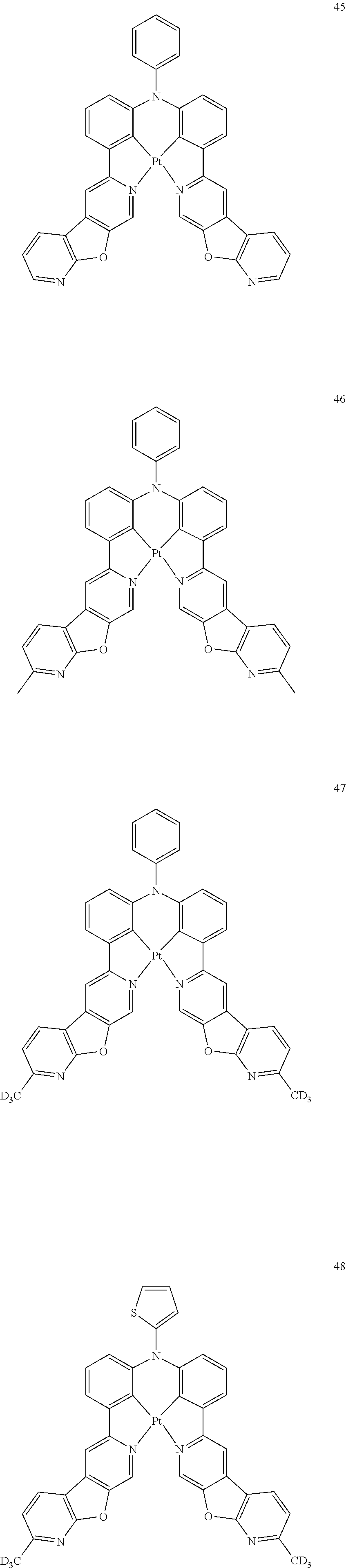

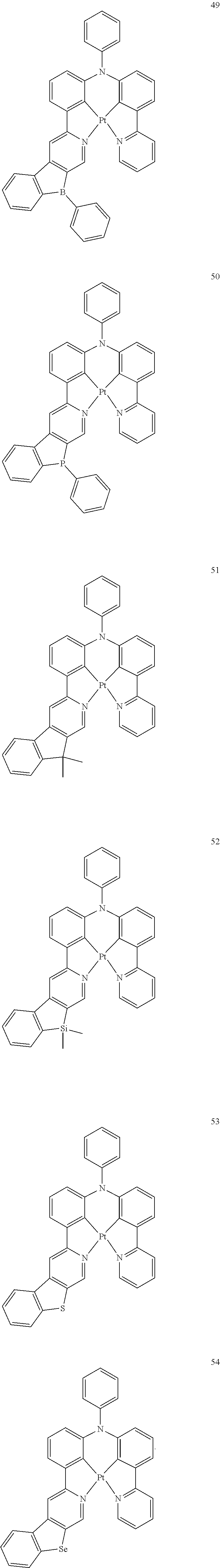

In Formula 1, X₁ may be N, and CY₁ may be selected from an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene-5-oxide group, an aza-9H-fluoren-9-one group, and an azadibenzothiophene-5,5-dioxide group, each including at least one N as a ring-forming atom. An organometallic compound including CY₁ as described above may have a long conjugation length, and thus a decay time may decrease. Therefore, an electronic device, e.g., an organic light-emitting device, including the organometallic compound may have improved luminous efficiency. Furthermore, the organometallic compound may have excellent heat resistance and thermal stability. Thus, processability in the manufacture of an electronic device, e.g., an organic light-emitting device, including the organometallic compound may improve. Accordingly, an electronic device, e.g., an organic light-emitting device, including the organometallic compound may have an improved lifespan. CY₁ in Formula 1 may contribute to the lowest unoccupied molecular orbital (LUMO) moiety of Formula 1, since X₁ is N. Due to the condensed ring structure of CY₁ that contributes to the LUMO moiety, the organometallic compound may have a suitable triplet energy level for use in an electronic device, e.g., an organic light-emitting device. In an embodiment, the organometallic compound represented by Formula 1 may have a suitable triplet energy level for use in red color emission with excellent color purity.

For example, the highest occupied molecular orbital (HOMO), LUMO, singlet (Si), and triplet (T₁) energy levels of the Compounds 2, 4, 5, 13, 14, 16, 21, 22, 37, 38, and A to C were evaluated by using a Gaussian according to a density functional theory (DFT) method (structure optimization was performed at a degree of B3LYP, and 6-31G(d, p)).

The results thereof are shown in Table 1.

TABLE 1

| | | | Energy | S₁ | T₁ |
|---|---|---|---|---|---|
| Com- | | | band | energy | energy |
| pound | HOMO | LUMO | gap | level | level |
| No. | (eV) | (eV) | (eV) | (eV) | (eV) |
| 2 | −4.731 | −1.819 | 2.552 | 2.056 | 1.895 |
| 4 | −4.379 | −1.818 | 2.561 | 2.066 | 1.902 |
| 5 | −4.502 | −1.905 | 2.597 | 2.094 | 1.929 |
| 13 | −4.382 | −1.76 | 2.622 | 2.108 | 1.929 |
| 14 | −4.457 | −1.855 | 2.602 | 2.097 | 1.934 |
| 16 | −4.352 | −1.752 | 2.6 | 2.091 | 1.92 |
| 21 | −4.481 | −1.885 | 2.596 | 2.093 | 1.929 |
| 22 | −4.384 | −1.797 | 2.587 | 2.099 | 1.942 |
| 37 | −4.415 | −1.877 | 2.538 | 1.976 | 1.779 |
| 38 | −4.338 | −1.732 | 2.606 | 2.088 | 1.914 |
| A | −4.414 | −1.566 | 2.848 | 2.243 | 1.983 |
| B | −4.527 | −1.647 | 2.880 | 2.270 | 2.013 |
| C | −4.430 | −1.579 | 2.851 | 2.243 | 1.988 |
| D | −5.109 | −1.654 | 3.455 | 2.766 | 2.394 |
| E | −5.302 | −1.788 | 3.514 | 2.829 | 2.499 |

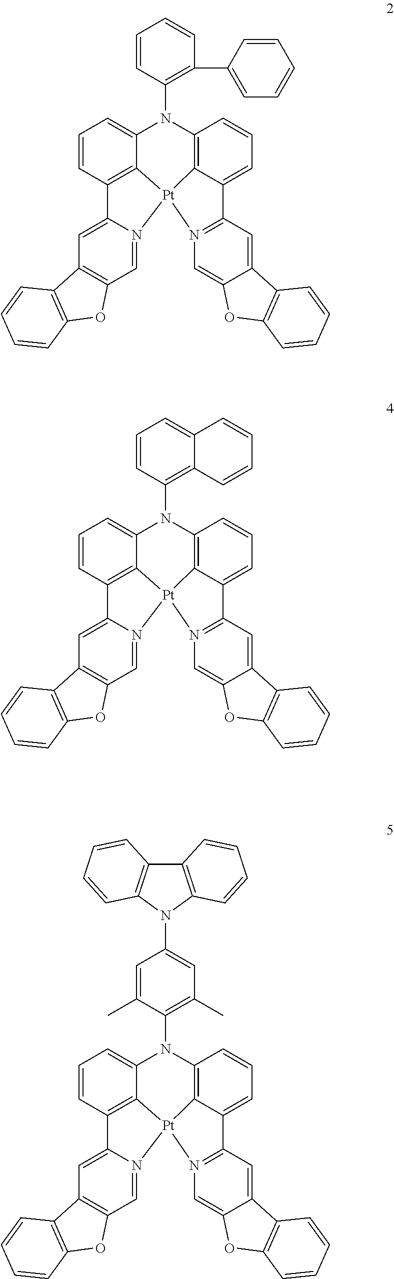

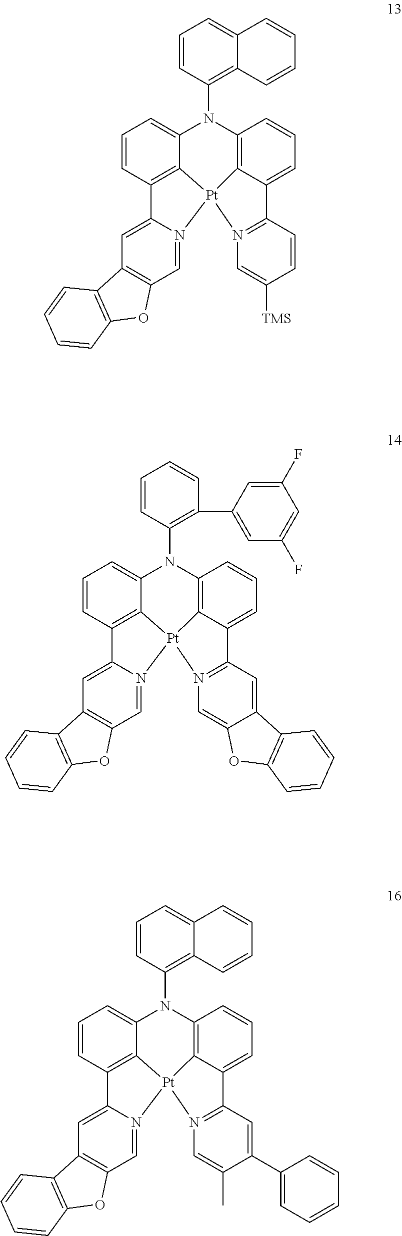

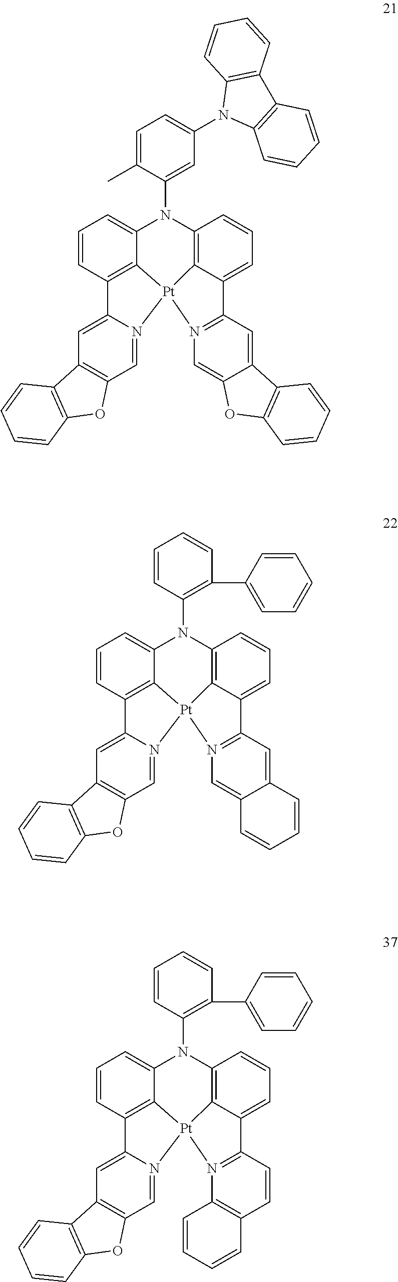

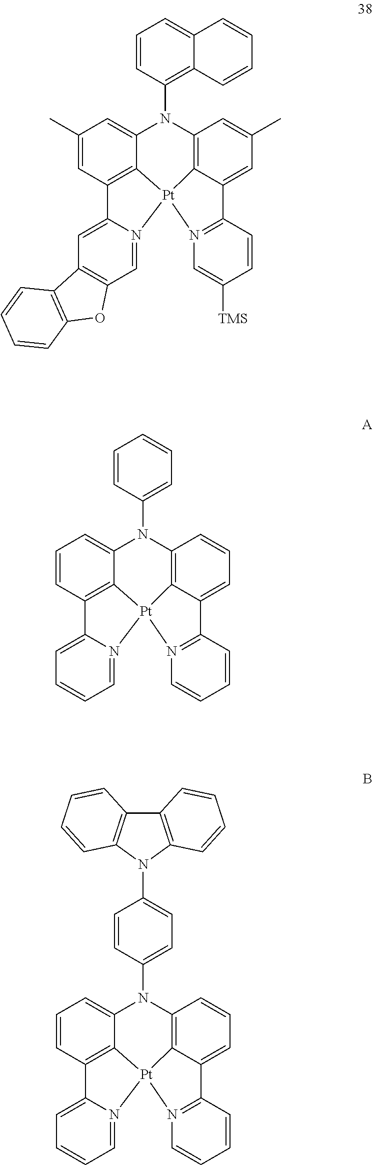

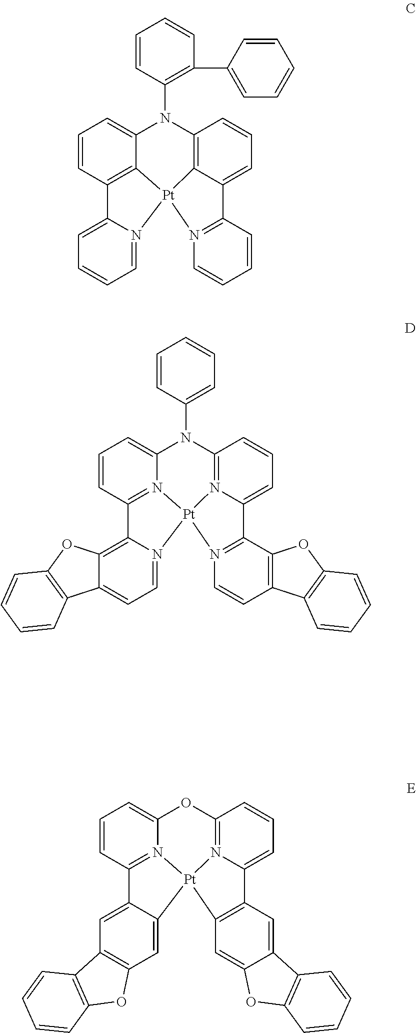

Referring to the results of Table 1, the organometallic compound of Formula 1 was found to have suitable electrical characteristics for use as a dopant in an electronic device, e.g., an organic light-emitting device.

A method of synthesizing the organometallic compound represented by Formula 1 may be apparent to one of ordinary skill in the art by referring to Synthesis Examples provided herein.

The organometallic compound represented by Formula 1 may be suitable for use in an organic layer of an organic light-emitting device, e.g., as a dopant in an emission layer of the organic layer. Thus, according to another aspect, there is provided an organic light-emitting device that may include a first electrode; a second electrode; and an organic layer disposed between the first electrode and the second electrode, wherein the organic layer includes an emission layer and at least one organometallic compound represented by Formula 1.

Since the organic light-emitting device has an organic layer including the organometallic compound represented by Formula 1, the organic light-emitting device may have a low driving voltage, high efficiency, high power, high quantum efficiency, long lifespan, low roll-off, and excellent color purity.

The organometallic compound represented by Formula 1 may be used in a pair of electrodes in an organic light-emitting device. For example, the organometallic compound represented by Formula 1 may be included in the emission layer. In this case, the organometallic compound may serve as a dopant and the emission layer may further include a host (that is, an amount of the organometallic compound represented by Formula 1 may be smaller than that of the host).

As used herein, “(for example, the organic layer) including at least one organometallic compound” means that “(the organic layer) including an organometallic compound of Formula 1, or at least two different organometallic compounds of Formula 1”.

For example, Compound 1 may only be included in the organic layer as an organometallic compound. In this embodiment, Compound 1 may be included in the emission layer of the organic light-emitting device. In some embodiments, Compounds 1 and 2 may be included in the organic layer as organometallic compounds. In this embodiment, Compounds 1 and 2 may both be included in the same layer (for example, both Compounds 1 and 2 may be included in the emission layer).

The first electrode may be anode, which is a hole injection electrode, and the second electrode may be a cathode, which is an electron injection electrode. In some embodiments, the first electrode is a cathode, which is an electron injection electrode, and the second electrode is an anode, which is a hole injection electrode.

For example, in the organic light-emitting device, the first electrode may be an anode, the second electrode may be a cathode, and the organic layer may further include a hole transport region disposed between the first electrode and the emission layer and an electron transport region disposed between the emission layer and the second electrode, wherein the hole transport region may include a hole injection layer, a hole transport layer, an electron blocking layer, or a combination thereof, and wherein the electron transport region may include a hole blocking layer, an electron transport layer, an electron injection layer, or a combination thereof.

The term “organic layer” as used herein refers to a single and/or a plurality of layers disposed between the first electrode and the second electrode in an organic light-emitting device. The “organic layer” may include not only organic compounds but also organometallic complexes including metals.

FIG. 1 illustrates a schematic view of an organic light-emitting device 10 according to an embodiment. Hereinafter, a structure of an organic light-emitting device according to one or more embodiments and a method of manufacturing the organic light-emitting device will be described with reference to FIG. 1. The organic light-emitting device 10 may include a first electrode 11, an organic layer 15, and a second electrode 19, which may be sequentially layered in the stated order.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

The first electrode 11 may be formed by vacuum-depositing or sputtering a material for forming the first electrode 11 on the substrate. The first electrode 11 may be an anode. The material for forming the first electrode 11 may be selected from materials with a high work function for easy hole injection. The first electrode 11 may be a reflective electrode, a semi-transmissive electrode, or a transmissive electrode.

The material for the first electrode 11 may be selected from indium tin oxide (ITO), indium zinc oxide (IZO), tin oxide (SnO₂), and zinc oxide (ZnO). In some embodiments, the material for forming the first electrode 11 may be a metal, such as magnesium (Mg), aluminum (AI), aluminum-lithium (Al—Li), calcium (Ca), magnesium-indium (Mg—In), or magnesium-silver (Mg—Ag).

The first electrode 11 may have a single-layered structure or a multi-layered structure including a plurality of layers. In some embodiments, the first electrode 11 may have a triple-layered structure of ITO/Ag/ITO, but embodiments are not limited thereto.

The organic layer 15 may be on the first electrode 11.

The organic layer 15 may include a hole transport region, an emission layer, and an electron transport region.

The hole transport region may be disposed between the first electrode 11 and the emission layer.

The hole transport region may include a hole injection layer, a hole transport layer, an electron blocking layer, a buffer layer, or a combination thereof.

The hole transport region may include a hole injection layer only or a hole transport layer only. In some embodiments, the hole transport region may include a hole injection layer and a hole transport layer which are sequentially stacked on the first electrode 11. In some embodiments, the hole transport region may include a hole injection layer, a hole transport layer, and an electron blocking layer, which are sequentially stacked on the first electrode 11.

When the hole transport region includes a hole injection layer, the hole injection layer may be formed on the first electrode 11 by using one or more suitable methods, such as vacuum deposition, spin coating, casting, and Langmuir-Blodgett (LB) deposition.

When a hole injection layer is formed by vacuum deposition, for example, the vacuum deposition may be performed at a deposition temperature in a range of about 100° C. to about 500° C., at a vacuum degree in a range of about 10⁻⁸ torr to about 10⁻³ torr, and at a deposition rate in a range of about 0 Angstroms per second (A/sec) to about 100 Å/sec, though the conditions may vary depending on a compound that is used as a hole injection material and a structure and thermal properties of a desired hole injection layer, but conditions for the vacuum deposition are not limited thereto.

When a hole injection layer is formed by spin coating, the spin coating may be performed at a coating rate in a range of about 2,000 revolutions per minute (rpm) to about 5,000 rpm, and at a temperature in a range of about 80° C. to 200° C. for removing a solvent after the spin coating, though the conditions may vary depending on a compound that is used as a hole injection material and a structure and thermal properties of a desired hole injection layer, but conditions for the spin coating are not limited thereto.

The conditions for forming a hole transport layer and an electron blocking layer may be inferred from the conditions for forming the hole injection layer.

The hole transport region may include at least one selected from m-MTDATA, TDATA, 2-TNATA, NPB, β-NPB, TPD, spiro-TPD, spiro-NPB, methylated-NPB, TAPC, HMTPD, 4,4′,4″-tris(N-carbazolyl)triphenylamine (TCTA), polyaniline/dodecylbenzene sulfonic acid (PANI/DBSA), poly(3,4-ethylenedioxythiophene)/poly(4-styrene sulfonate) (PEDOT/PSS), polyaniline/camphor sulfonic acid (PANI/CSA), (polyaniline)/poly(4-styrene sulfonate) (PANI/PSS), a compound represented by Formula 201, and a compound represented by Formula 202:

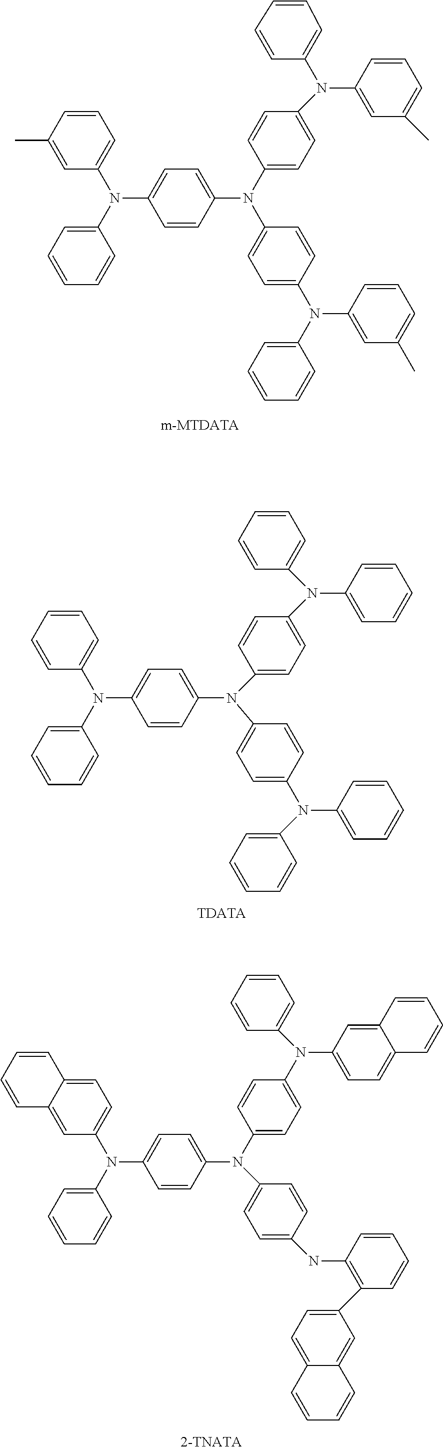

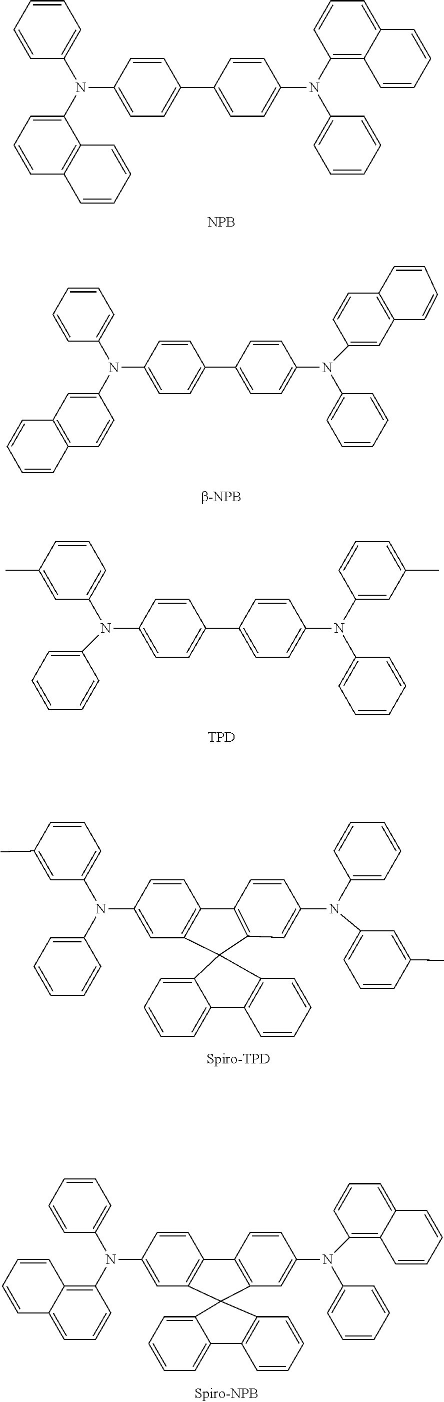

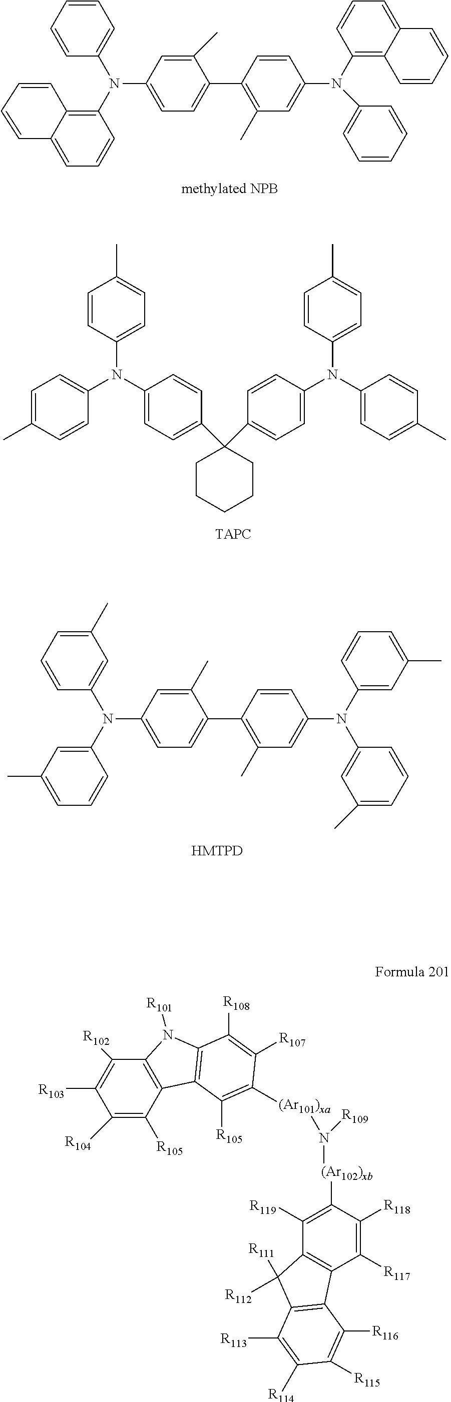

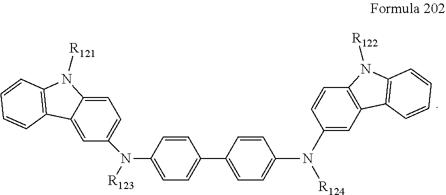

In Formula 201, Ar₁₀₁ and Ar₁₀₂ may each independently be selected from:

a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group; and a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

In Formula 201, xa and xb may each independently be an integer from 0 to 5. In some embodiments, xa and xb may each independently be an integer of 0, 1, or 2. In some embodiments, xa may be 1, and xb may be 0, but embodiments are not limited thereto.

In Formulae 201 and 202, R₁₀₁ to R₁₀₈, R₁₁₁ to R₁₁₉, and R₁₂₁ to R₁₂₄ may each independently be selected from:

hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (e.g., a methyl group, an ethyl group, a propyl group, a butyl group, pentyl group, or a hexyl group), and a C₁-C₁₀ alkoxy group (e.g., a methoxy group, an ethoxy group, a propoxy group, a butoxy group, or a pentoxy group);

a C₁-C₁₀ alkyl group and a C₁-C₁₀ alkoxy group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, and a phosphoric acid group or a salt thereof;

a phenyl group, a naphthyl group, an anthracenyl group, a fluorenyl group, and a pyrenyl group; and

a phenyl group, a naphthyl group, an anthracenyl group, a fluorenyl group, and a pyrenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, and a C₁-C₁₀ alkoxy group, but embodiments are not limited thereto.

In Formula 201, R₁₀₉ may be selected from:

a phenyl group, a naphthyl group, an anthracenyl group, and a pyridinyl group; and

a phenyl group, a naphthyl group, an anthracenyl group, and a pyridinyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, and a pyridinyl group.

In some embodiments, the compound represented by Formula 201 may be represented by Formula 201A, but embodiments are not limited thereto:

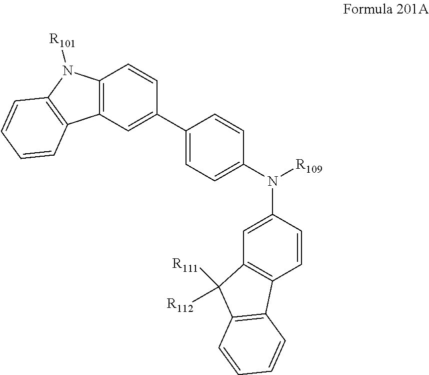

R₁₀₁, R₁₁₁, R₁₁₂, and R₁₀₉ in Formula 201A may be the same as those described above.

In some embodiments, the compounds represented by Formulae 201 and 202 may include Compounds HT1 to HT20, but embodiments are not limited thereto:

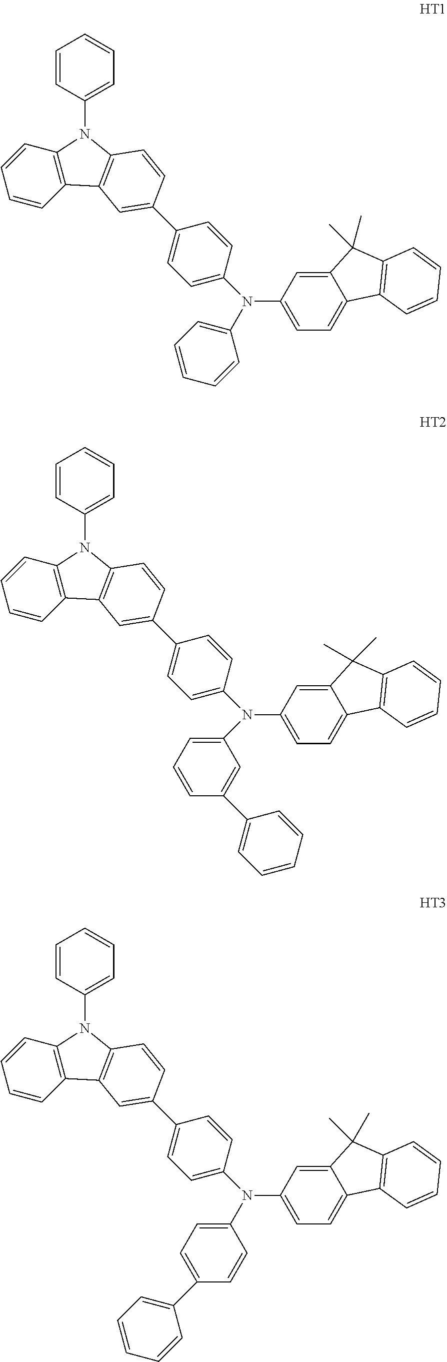 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

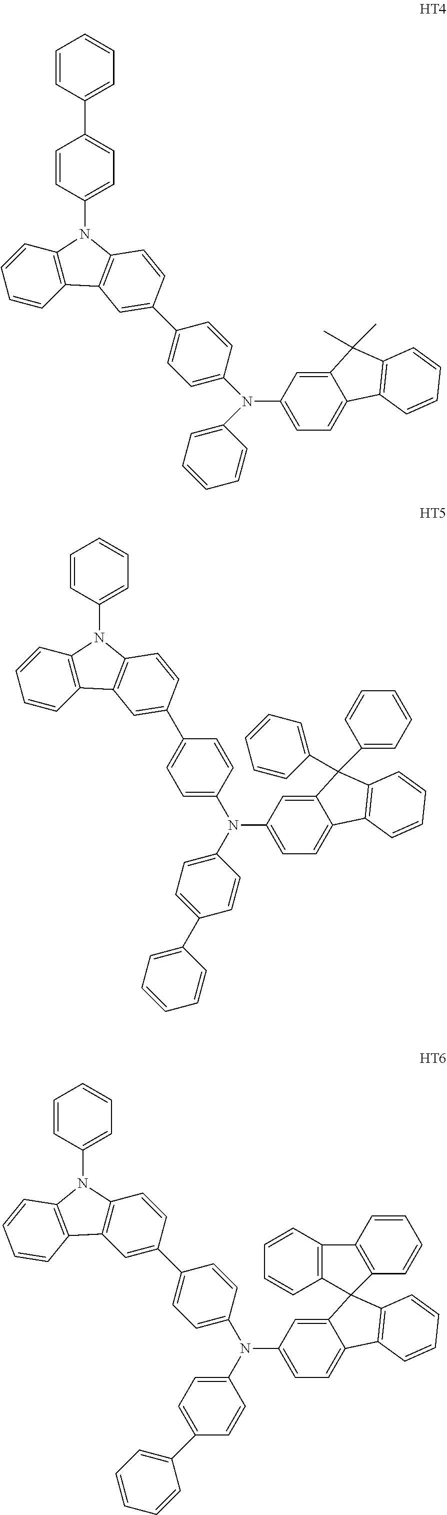 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

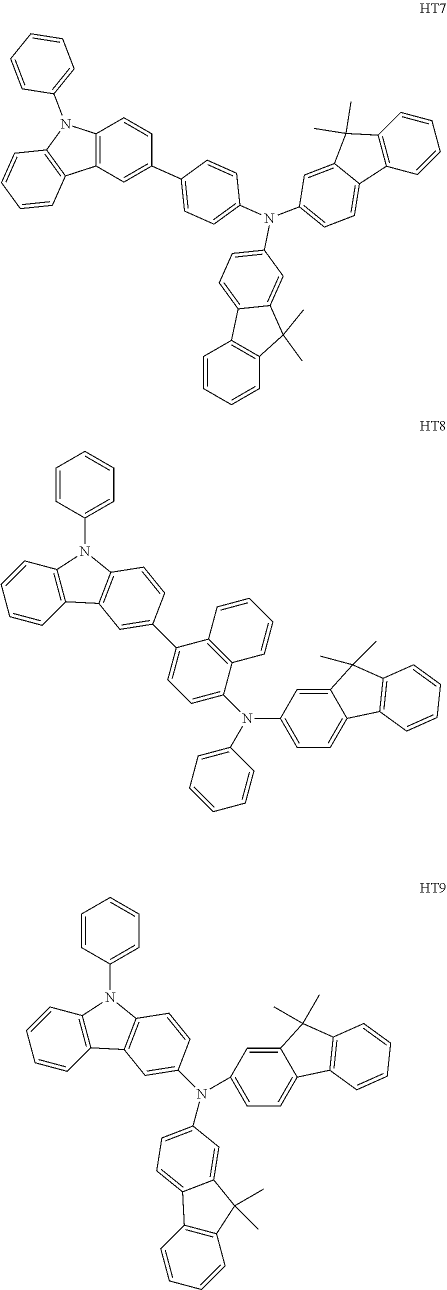 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

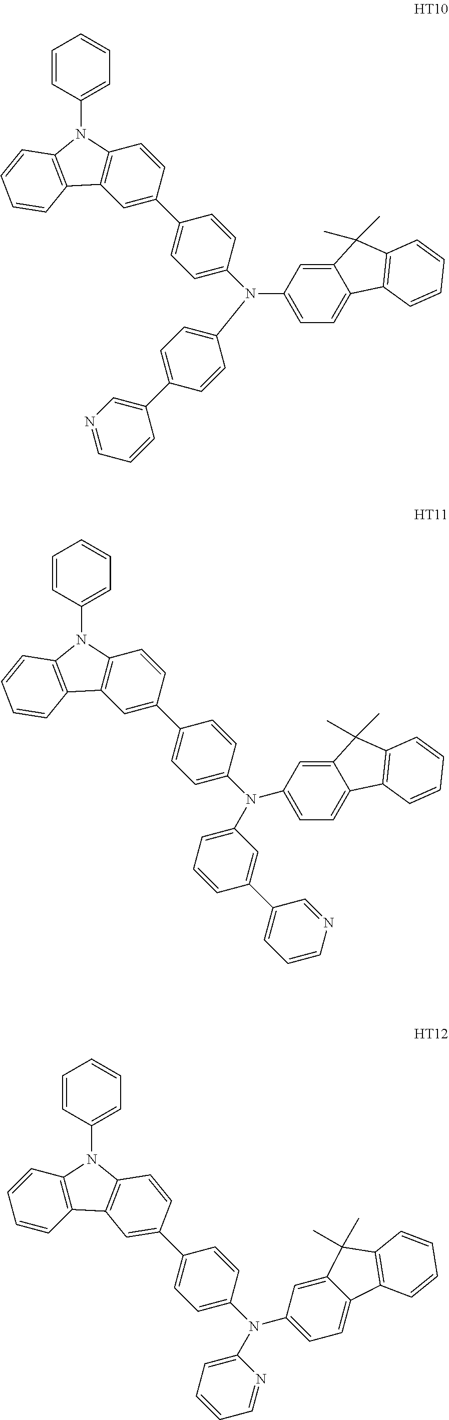 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

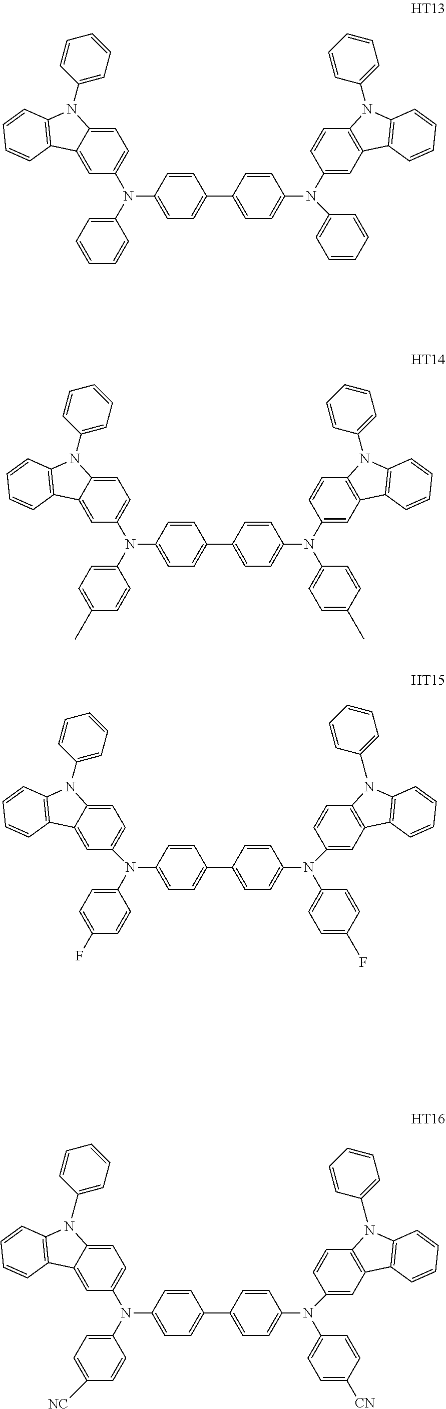 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

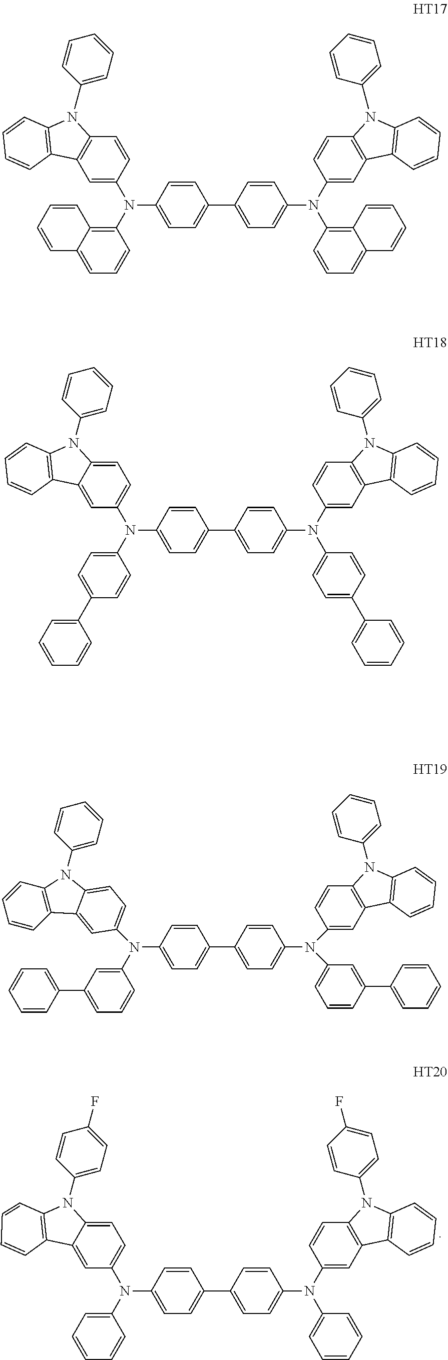 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

The thickness of the hole transport region may be in a range of about 100 (Angstroms) Å to about 10,000 Å, and in some embodiments, about 100 Å to about 1,000 Å. When the hole transport region includes at least one of a hole injection layer and a hole transport layer, the thickness of the hole injection layer may be in a range of about 100 Å to about 10,000 Å, and in some embodiments, about 100 Å to about 1,000 Å, and the thickness of the hole transport layer may be in a range of about 50 Å to about 2,000 Å, and in some embodiments, about 100 Å to about 1,500 Å. While not wishing to be bound by theory, it is understood that when the thicknesses of the hole transport region, the hole injection layer, and the hole transport layer are within any of these ranges, excellent hole transport characteristics may be obtained without a substantial increase in driving voltage.

The hole transport region may include a charge generating material as well as the aforementioned materials, to improve conductive properties of the hole transport region. The charge generating material may be substantially homogeneously or non-homogeneously dispersed in the hole transport region.

The charge generating material may include, for example, a p-dopant. The p-dopant may include one of a quinone derivative, a metal oxide, and a compound containing a cyano group, but embodiments are not limited thereto. For example, non-limiting examples of the p-dopant include a quinone derivative, such as tetracyanoquinodimethane (TCNQ) or 2,3,5,6-tetrafluoro-tetracyano-1,4-benzoquinonedimethane (F4-TCNQ); a metal oxide, such as a tungsten oxide or a molybdenum oxide; and a compound containing a cyano group, such as Compound HT-D1, but embodiments are not limited thereto:

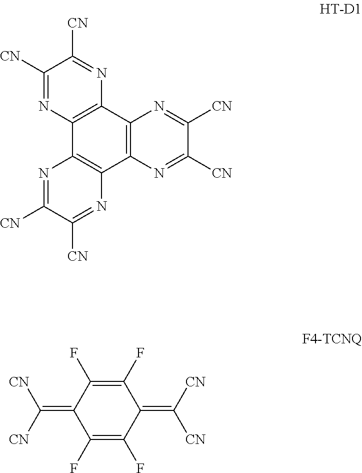

The hole transport region may further include a buffer layer.

The buffer layer may compensate for an optical resonance distance depending on a wavelength of light emitted from the emission layer to improve the efficiency of an organic light-emitting device.

An emission layer may be formed on the hole transport region by using one or more suitable methods, such as vacuum deposition, spin coating, casting, or LB deposition. When the emission layer is formed by vacuum deposition or spin coating, vacuum deposition and coating conditions for the emission layer may be generally similar to the conditions for forming a hole injection layer, though the conditions may vary depending on a compound that is used.

When the hole transport region includes an electron blocking layer, a material for forming the electron blocking layer may be selected from the materials for forming a hole transport region and host materials described herein, but embodiments are not limited thereto. In some embodiments, when the hole transport region includes an electron blocking layer, mCP described herein may be used for forming the electron blocking layer.

The emission layer may include a host and a dopant, and the dopant may include the organometallic compound represented by Formula 1.

The host may include at least one selected from TPBi, TBADN, ADN (also known as “DNA”), CBP, CDBP, TCP, mCP, and Compounds H50 and H51:

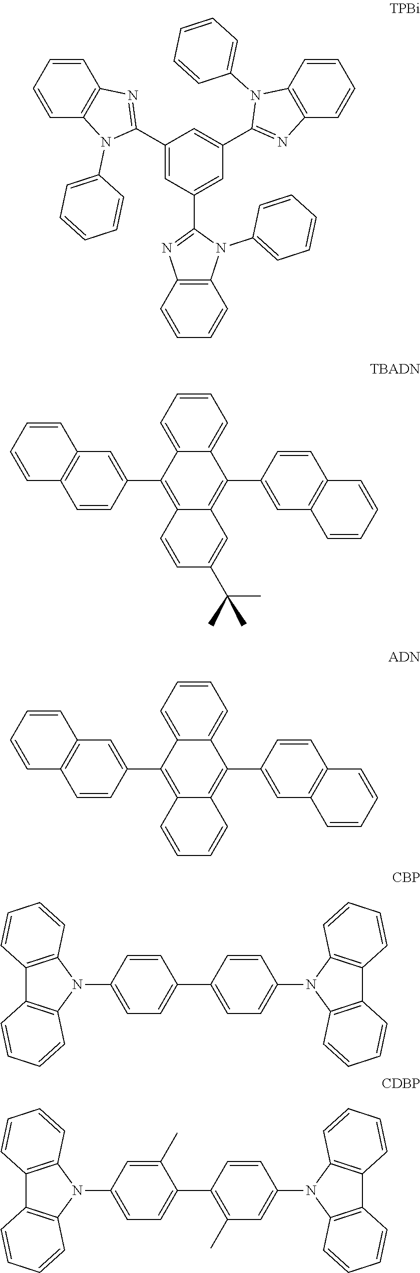

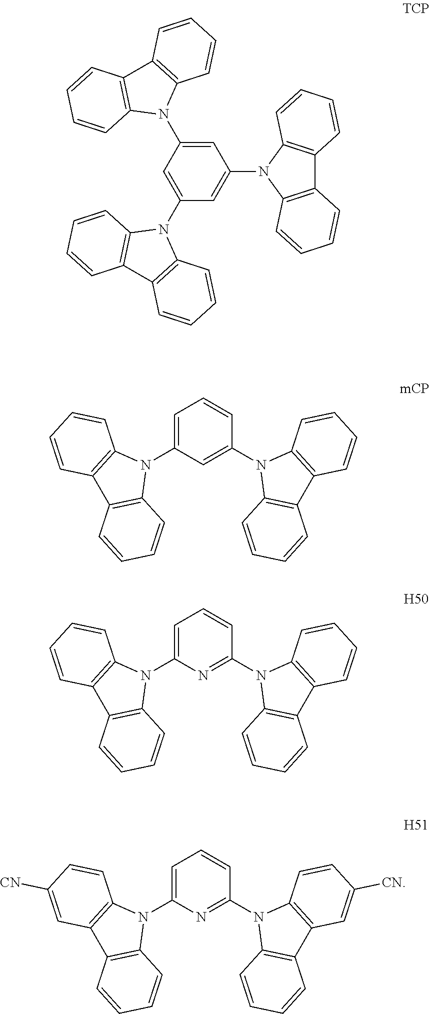

In some embodiments, the host may further include a compound represented by Formula 301:

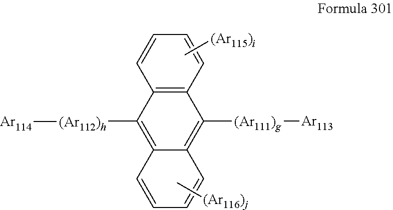

wherein, in Formula 301, Ar₁₁₁ and Ar₁₁₂ may each independently be selected from:

a phenylene group, a naphthylene group, a phenanthrenylene group, and a pyrenylene group; and

a phenyl group, a phenylene group substituted with at least one selected from a naphthyl group and an anthracenyl group, a naphthylene group, a phenanthrenylene group, and a pyrenylene group.

In Formula 301, Ar₁₁₃ to Ar₁₁₆ may each independently be selected from:

a C₁-C₁₀ alkyl group, a phenyl group, a naphthyl group, a phenanthrenyl group, and a pyrenyl group; and

a phenyl group, a phenyl group substituted with at least one selected from a naphthyl group and an anthracenyl group, a naphthyl group, a phenanthrenyl group, and a pyrenyl group.

In Formula 301, g, h, i, and j may each independently be an integer from 0 to 4. For example, g, h, i, and j may each independently be 0, 1, or 2.

In Formula 301, Ar₁₁₃ to Ar₁₁₆ may each independently be selected from:

a C₁-C₁₀ alkyl group substituted with at least one selected from a phenyl group, a naphthyl group, and an anthracenyl group;

a phenyl group, a naphthyl group, an anthracenyl group, a pyrenyl group, a phenanthrenyl group, and a fluorenyl group; and

a phenyl group, a naphthyl group, an anthracenyl group, a pyrenyl group, a phenanthrenyl group, and a fluorenyl group, each substituted with at least one selected from deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a phenyl group, a naphthyl group, an anthracenyl group, a pyrenyl group, a phenanthrenyl group, and a fluorenyl group; and

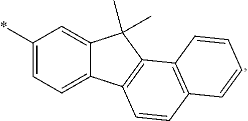 [SMILES: *c1ccc2c(c1)C(C)(C)c1c-2ccc2ccccc12]

but embodiments are not limited thereto.

In some embodiments, the host may include a compound represented by Formula 302:

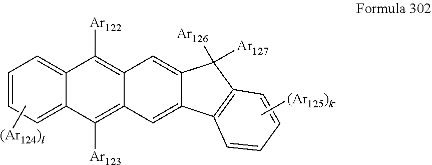

In Formula 302, Ar₁₂₂ to Ar₁₂₅ may be the same as described herein with reference to Ar₁₁₃ in Formula 301.

In Formula 302, Ar₁₂₆ and Ar₁₂₇ may each independently be a C₁-C₁₀ alkyl group, e.g., a methyl group, an ethyl group, or a propyl group.

In Formula 302, k and l may each independently be an integer from 0 to 4. In some embodiments, k and l may each be 0, 1, or 2.

In some embodiments, the compounds represented by Formulae 301 and 302 may each include at least one of Compounds H1 to H42, but embodiments are not limited thereto:

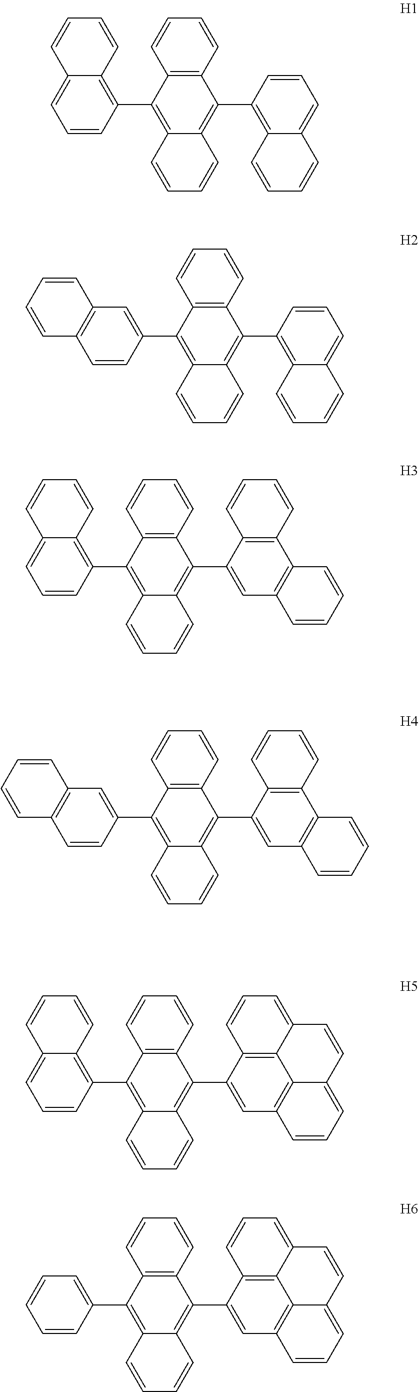

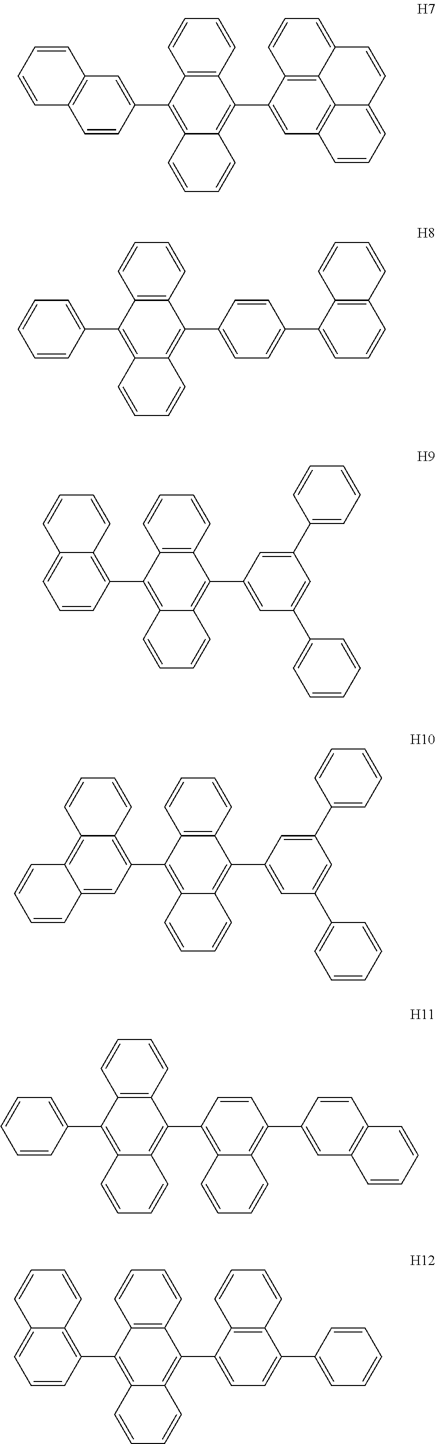

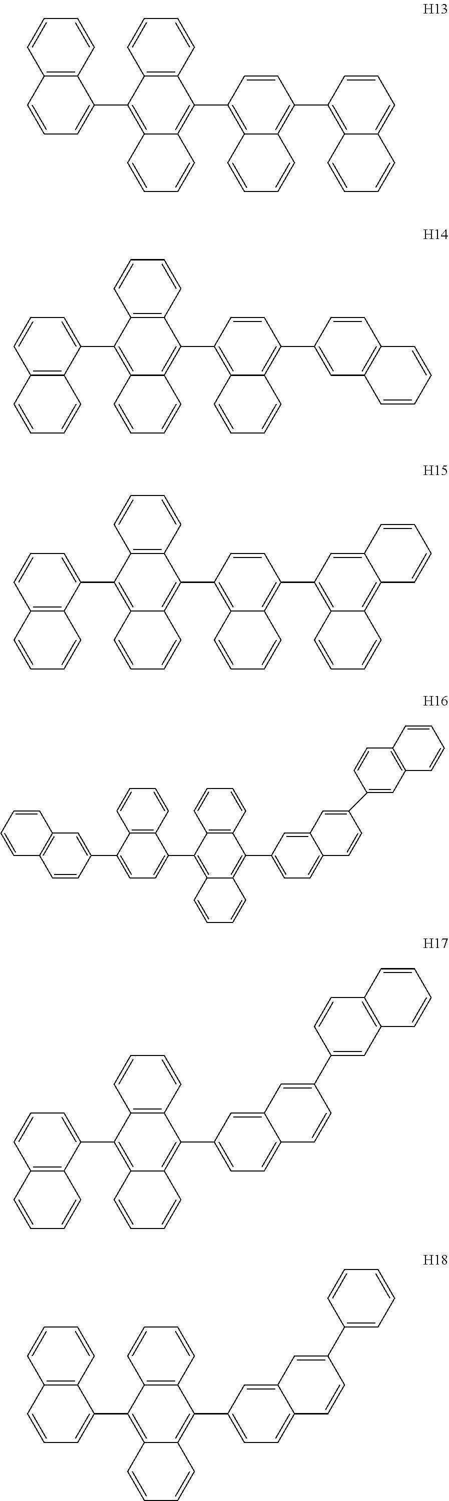

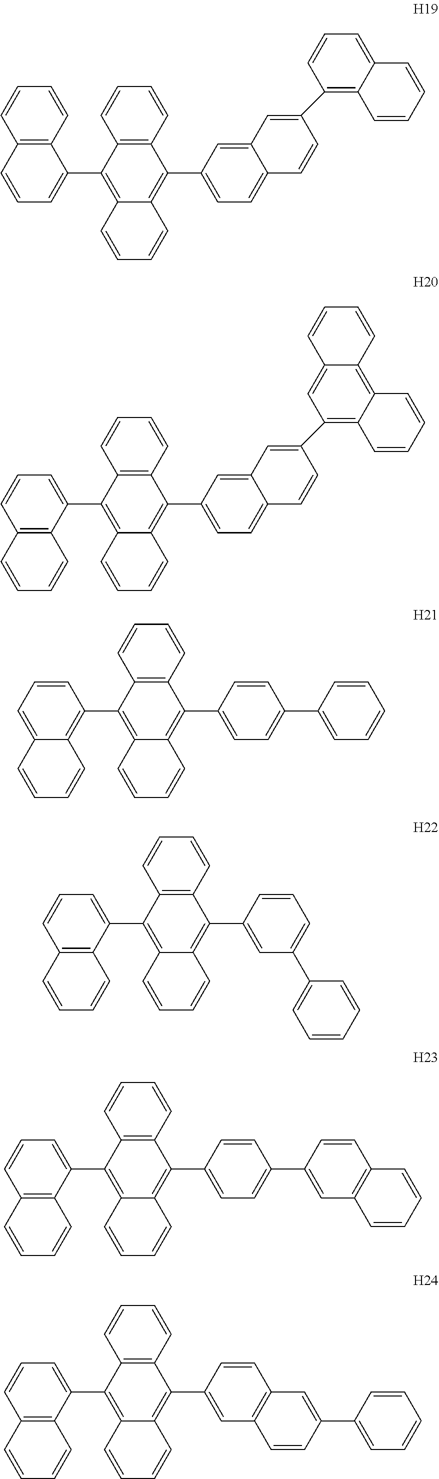

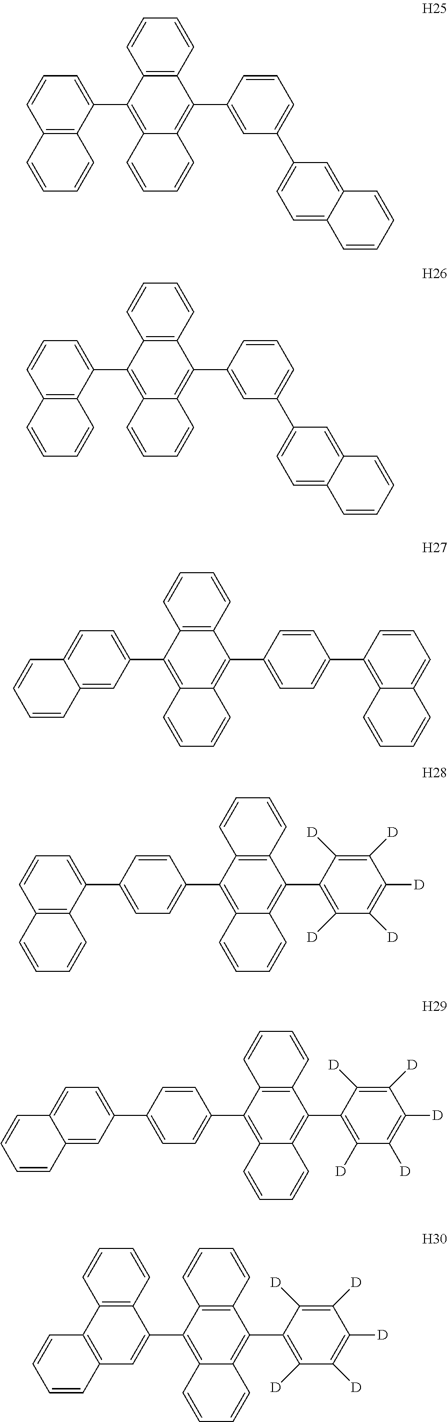

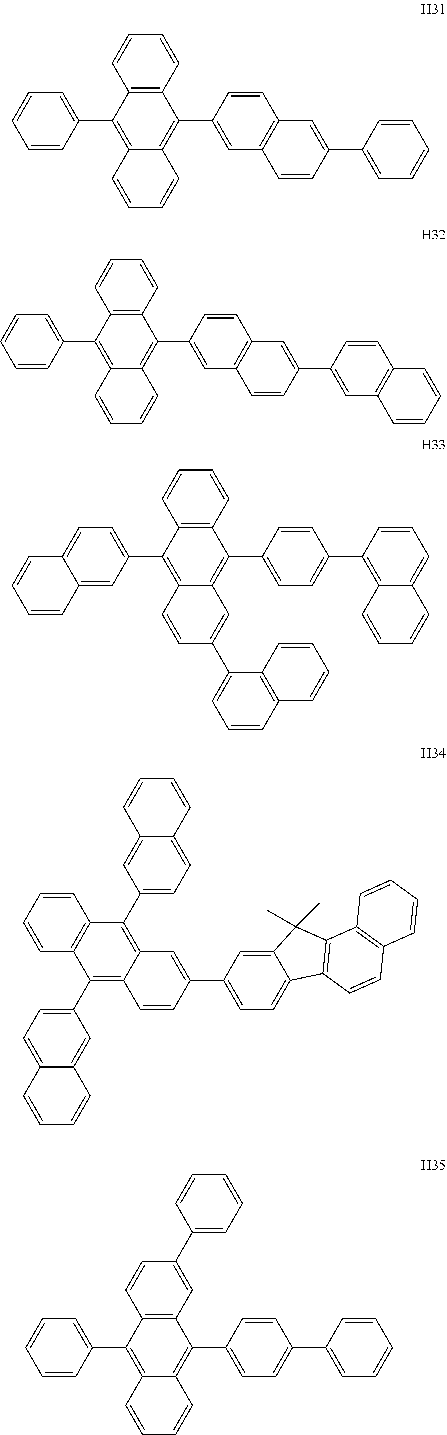

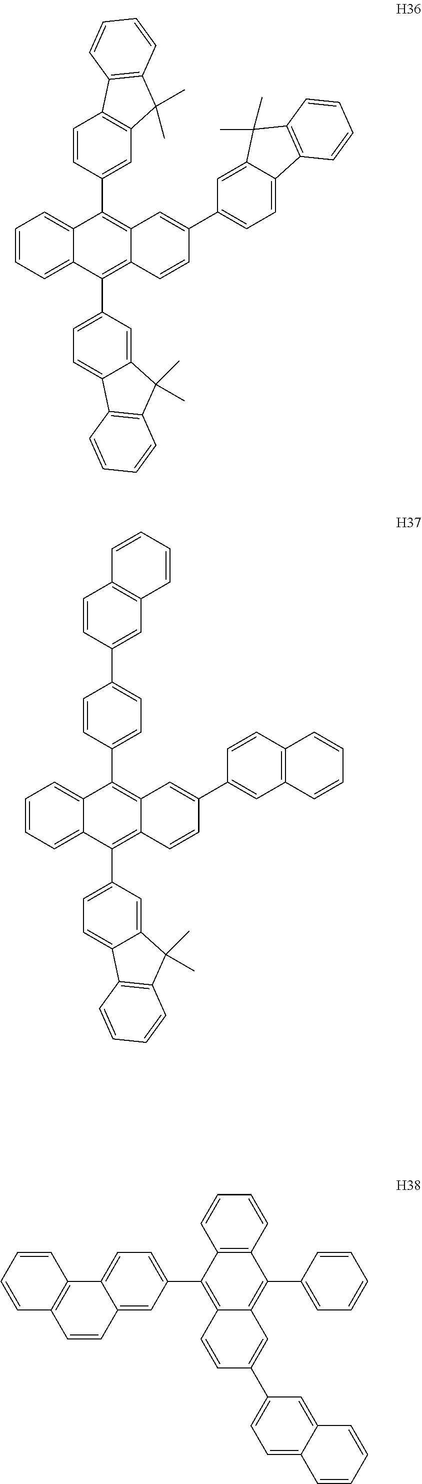

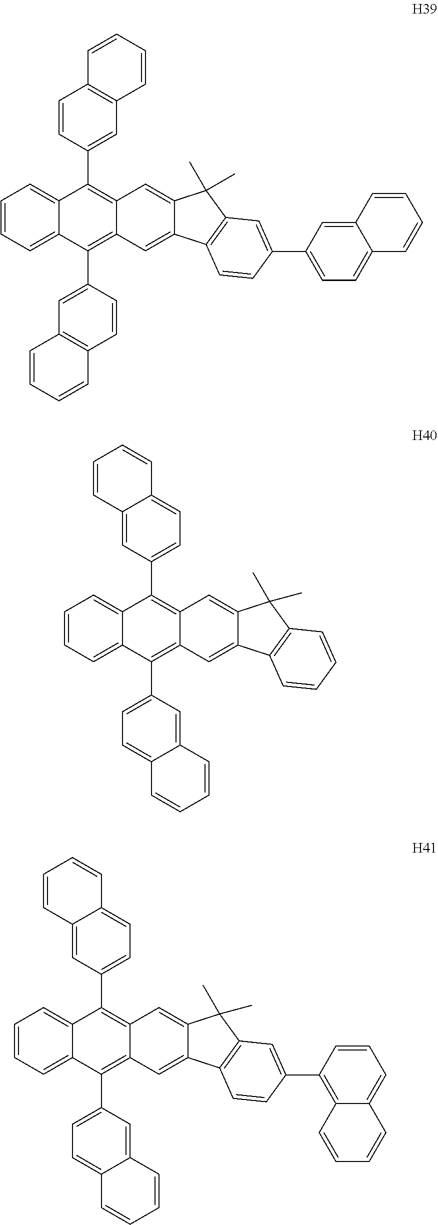

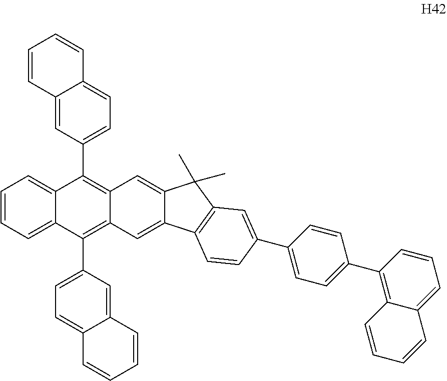 [SMILES: CC1(C)c2cc(-c3ccc(-c4cccc5ccccc45)cc3)ccc2-c2cc3c(-c4ccc5ccccc5c4)c4ccccc4c(-c4ccc5ccccc5c4)c3cc21]

When the organic light-emitting device 10 is a full-color organic light-emitting device, the emission layer may be patterned into a red emission layer, a green emission layer, and a blue emission layer. In some embodiments, the emission layer may have a structure in which the red emission layer, the green emission layer, and/or the blue emission layer are layered to emit white light. In some embodiments, the structure of the emission layer may vary.

When the emission layer includes the host and the dopant, an amount of the dopant may be selected from a range of about 0.01 parts to about 15 parts by weight based on about 100 parts by weight of the host, but embodiments are not limited thereto.

The thickness of the emission layer may be in a range of about 100 Å to about 1,000 Å, and in some embodiments, about 200 Å to about 600 Å. While not wishing to be bound by theory, it is understood that when the thickness of the emission layer is within any of these ranges, improved luminous characteristics may be obtained without a substantial increase in driving voltage.

Next, an electron transport region may be formed on the emission layer.

The electron transport region may include a hole blocking layer, an electron transport layer, an electron injection layer, or a combination thereof.

In some embodiments, the electron transport region may have a hole blocking layer/an electron transport layer/an electron injection layer structure or an electron transport layer/an electron injection layer structure, but embodiments are not limited thereto. The electron transport layer may have a single-layered structure or a multi-layered structure including two or more different materials.

The conditions for forming a hole blocking layer, an electron transport layer, and an electron injection layer may be inferred based on the conditions for forming the hole injection layer.

When the electron transport region includes a hole blocking layer, the hole blocking layer may include, for example, at least one selected from BCP, Bphen, and BAlq, but embodiments are not limited thereto:

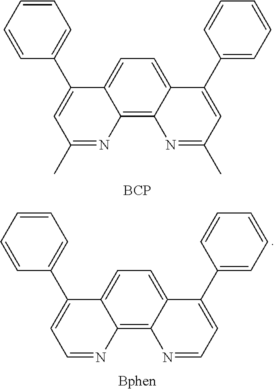

The thickness of the hole blocking layer may be in a range of about 20 Å to about 1,000 Å, and in some embodiments, about 30 Å to about 300 Å. While not wishing to be bound by theory, it is understood that when the thickness of the hole blocking layer is within these ranges, excellent hole blocking characteristics may be obtained without a substantial increase in driving voltage.

The electron transport layer may further include at least one selected from BCP, BPhen, Alq3, BAlq, TAZ, and NTAZ:

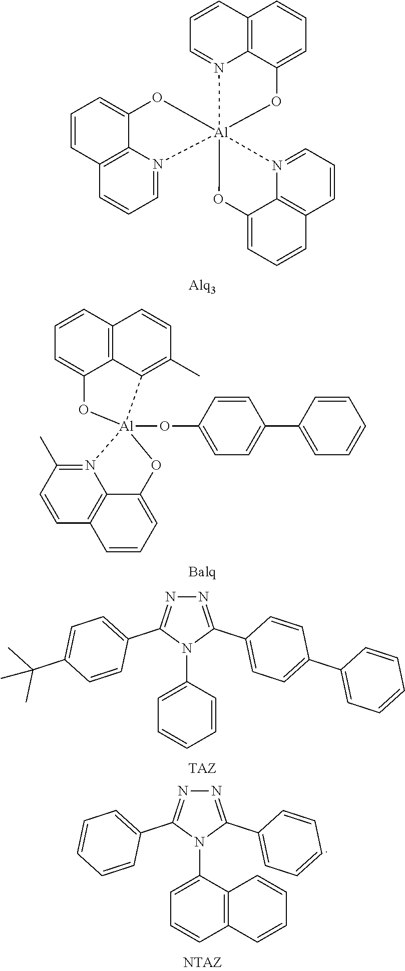

In some embodiments, the electron transport layer may include at least one selected from Compounds ET1 and ET2, but embodiments are not limited thereto:

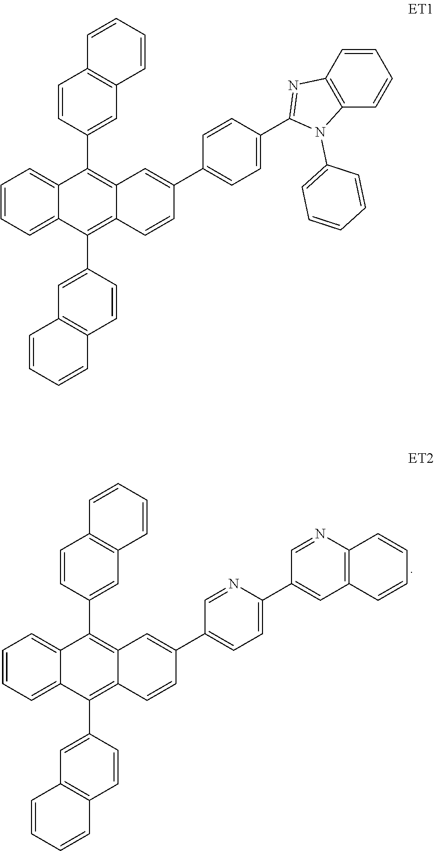 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1]

The thickness of the electron transport layer may be in a range of about 100 Å to about 1,000 Å, and in some embodiments, about 150 Å to about 500 Å. While not wishing to be bound by theory, it is understood that when the thickness of the electron transport layer is within any of these ranges, excellent electron transport characteristics may be obtained without a substantial increase in driving voltage.

The electron transport layer may further include a material containing metal, in addition to the materials described above.

The material containing metal may include a lithium (Li) complex. The Li complex may include, e.g., Compound ET-D1 (lithium quinolate, LiQ) or Compound ET-D2:

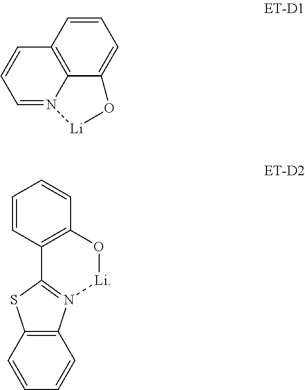

The electron transport region may include an electron injection layer that facilitates electron injection from the second electrode 19.

The electron injection layer may include at least one selected from LiF, NaCl, CsF, Li₂O, and BaO.

The thickness of the electron injection layer may be in a range of about 1 Å to about 100 Å, for example, about 3 Å to about 90 Å. While not wishing to be bound by theory, it is understood that when the thickness of the electron injection layer is within any of these ranges, excellent electron injection characteristics may be obtained without a substantial increase in driving voltage.

The second electrode 19 may be formed on the organic layer 15. The second electrode 19 may be a cathode. A material for forming the second electrode 19 may be a material with a relatively low work function, such as a metal, an alloy, an electrically conductive compound, and a mixture thereof. Examples of the material for forming the second electrode 19 may include lithium (Li), magnesium (Mg), aluminum (AI), aluminum-lithium (Al—Li), calcium (Ca), magnesium-indium (Mg—In), and magnesium-silver (Mg—Ag). In some embodiments, ITO or IZO may be used to form a transmissive second electrode 19 to manufacture a top emission light-emitting device. In some embodiments, the material used to form the second electrode 19 may vary.

Hereinbefore the organic light-emitting device 10 has been described with reference to FIG. 1, but embodiments are not limited thereto.

According to an aspect of another embodiment, a diagnostic composition includes at least one organometallic compound represented by Formula 1.

Since the organometallic compound represented by Formula 1 provides high luminous efficiency, the diagnostic efficiency of the diagnostic composition that includes the organometallic compound represented by Formula 1 may be excellent.

The diagnostic composition may be applied in various ways, such as in a diagnostic kit, a diagnostic reagent, a biosensor, or a biomarker.

The term “C₁-C₆₀ alkyl group” as used herein refers to a linear or branched saturated aliphatic hydrocarbon monovalent group having 1 to 60 carbon atoms.

Examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is a C₁-C₆₀ alkyl group). Examples thereof include a methoxy group, an ethoxy group, and an iso-propyloxy (iso-propoxy) group.

The term “C₂-C₆₀ alkenyl group” as used herein refers to a group formed by including at least one carbon-carbon double bond in the middle or at the terminus of the C₂-C₆₀ alkyl group. Examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a group formed by including at least one carbon-carbon triple bond in the middle or at the terminus of the C₂-C₆₀ alkyl group. Examples thereof include an ethenyl group and a propenyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent monocyclic saturated hydrocarbon group including 3 to 10 carbon atoms. Examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent monocyclic group including at least one heteroatom selected from N, O, P, Si, and S as a ring-forming atom and 1 to 10 carbon atoms. Examples thereof include a tetrahydrofuranyl group and a tetrahydrothiophenyl group. The term “C₁-C₁₀ heterocycloalkylene group” as used herein refers to a divalent group having the same structure as the C₁-C₆₀ heterocycloalkyl group.

The term “C₃-C₁₀ cycloalkenyl group” as used herein refers to a monovalent monocyclic group including 3 to 10 carbon atoms and at least one carbon-carbon double bond in its ring, wherein the molecular structure as a whole is non-aromatic.

Examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group including at least one heteroatom selected from N, O, P, Si, and S as a ring-forming atom, 1 to 10 carbon atoms, and at least one double bond in its ring. Examples of the C₁-C₁₀ heterocycloalkenyl group include a 2,3-dihydrofuranyl group and a 2,3-dihydrothiophenyl group. The term “C₁-C₁₀ heterocycloalkylene group” as used herein refers to a divalent group having the same structure as the C₁-C₁₀ heterocycloalkyl group.

The term “C₆-C₆₀ aryl group” as used herein refers to a monovalent group having a carbocyclic aromatic system having 6 to 60 carbon atoms. The term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and a C₆-C₆₀ arylene group each include at least two rings, the at least two rings may be fused.

The term “C₁-C₆₀ heteroaryl group” as used herein refers to a monovalent group having a cyclic aromatic system having at least one heteroatom selected from N, O, P, Si, and S as a ring-forming atom and 1 to 60 carbon atoms. The term “C₁-C₆₀ heteroarylene group” as used herein refers to a divalent group having a cyclic aromatic system having at least one heteroatom selected from N, O, P, and S as a ring-forming atom and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include at least two rings, the at least two rings may be fused.

The term “C₆-C₆₀ aryloxy group” as used herein indicates —OA₁₀₂ (wherein A₁₀₂ is a C₆-C₆₀ aryl group). The term “C₆-C₆₀ arylthio group” as used herein indicates —SA₁₀₃ (wherein A₁₀₃ is a C₆-C₆₀ aryl group).

The term “monovalent non-aromatic condensed polycyclic group” as used herein refers to a monovalent group that has two or more condensed rings and only carbon atoms (e.g., the number of carbon atoms may be in a range of 8 to 60) as ring-forming atoms, wherein the molecular structure as a whole is non-aromatic. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group that has two or more condensed rings and a heteroatom selected from N, O, P, Si, and S and carbon atoms (e.g., the number of carbon atoms may be in a range of 1 to 60) as ring-forming atoms, wherein the molecular structure as a whole is non-aromatic. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₃₀ carbocyclic group” as used herein refers to a saturated or unsaturated cyclic group including 5 to 30 carbon atoms only as ring-forming atoms. The C₅-C₃₀ carbocyclic group may be a monocyclic group or a polycyclic group.

The term “C₁-C₃₀ heterocyclic group” as used herein refers to saturated or unsaturated cyclic group including 1 to 30 carbon atoms and at least one heteroatom selected from N, O, P, Si, and S as ring-forming atoms. The C₁-C₃₀ heterocyclic group may be a monocyclic group or a polycyclic group.

At least one substituent of the substituted C₅-C₃₀ carbocyclic group, substituted C₂-C₃₀ heterocyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and

—N(Q₃₁)(Q₃₂), —Si(Q₃₃)(Q₃₄)(Q₃₅), —B(Q₃₆)(Q₃₇), and —P(═O)(Q₃₈)(Q₃₉),

wherein Q₁ to Q₉, Q₁₁ to Q₁₉, Q₂₁ to Q₂₉, and Q₃₁ to Q₃₉ may each independently be selected from a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ alkyl group, and a C₆-C₆₀ aryl group.

When a group containing a specified number of carbon atoms is substituted with any of the groups listed in the preceding paragraph, the number of carbon atoms in the resulting “substituted” group is defined as the sum of the carbon atoms contained in the original (unsubstituted) group and the carbon atoms (if any) contained in the substituent.

For example, when the term “substituted C₁-C₃₀ alkyl” refers to a C₁-C₃₀ alkyl group substituted with C₆-C₃₀ aryl group, the total number of carbon atoms in the resulting aryl substituted alkyl group is C₇-C₆₀.

Hereinafter, a compound and an organic light-emitting device according to an embodiment will be described in detail with reference to Synthesis Examples and Examples, however, the present disclosure is not limited thereto. The wording “B was used instead of A” used in describing Synthesis Examples means that an amount of B used was identical to an amount of A used in terms of molar equivalents.

### EXAMPLES

### Synthesis Example 1: Synthesis of Compound 1

### Synthesis of Intermediate 1-a

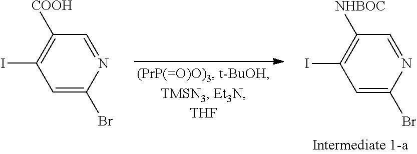

18.3 grams (g) 18.3 g (55.7 mmol) of 6-bromo-4-iodo-nicotinic acid, 18 mL (83.6 mmol) of diphenylphosphoryl azide, 23.5 mL (167.2 mmol) of trimethylamine, 110 mL of tert-butanol, and 120 mL of toluene were added to a 500 milliliters (mL) round-bottom flask, and the mixture was allowed to react at a temperature of 110° C. for 5 hours. The resultant was cooled to room temperature to complete the reaction. The solvent was removed under reduced pressure, the product was extracted using ethyl acetate and water and dried using Na₂SO₄. The resultant thus obtained was concentrated under reduced pressure to obtain a solid, which was then washed with methanol and diethyl ether and dried, thereby obtaining a white solid (i.e., Intermediate 1-a) (yield: 70%).

LCMS: m/z calcd for C₁₀H₁₂BrIN₂O₂ 397.91; Found 399.0.

### Synthesis of Intermediate 1-b

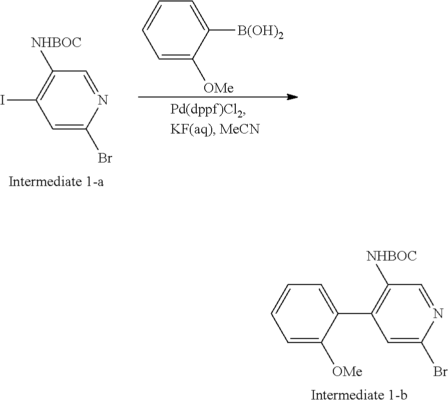

3.2 g (8.0 mmol) of Intermediate 1-a, 1.8 g (12.0 mmol) of (2-methoxyphenyl)boronic acid, 0.58 g (0.8 mmol) of (Pd(dppf)Cl₂ (1,1′-[bis(diphenylphosphine)ferrocene]dichloropalladium(II), and 1.2 g (20.0 mmol) of potassium fluoride were added to a 500 mL round-bottom flask, and mixed with 70 mL of acetonitrile. The mixture was allowed to react at a temperature of 90° C. for 6 hours.

The resultant was cooled to room temperature, and water was added thereto to complete the reaction. The product was extracted therefrom using dichloromethane and dried using Na₂SO₄. The resultant thus obtained was concentrated under reduced pressure to obtain a solid, which was purified through column chromatography, thereby obtaining Intermediate 1-b (yield: 72%).

LCMS: m/z calcd for C₁₇H₁₉BrN₂O₃ 378.06; Found 379.

### Synthesis of Intermediate 1-c

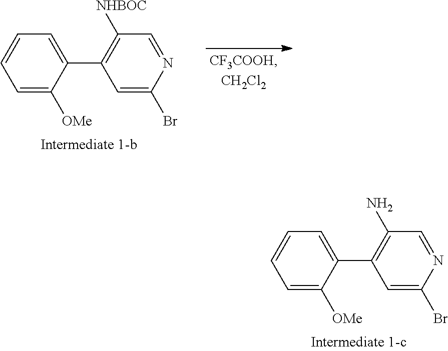

12.9 g (34.1 mmol) of Intermediate 1-b was mixed with 150 mL of dichloromethane in a 500 mL of round-bottom flask, and 50 mL of trifluoroacetic acid was added thereto. The mixture was then allowed to react at ambient temperature for 2 hours. A solvent was removed under reduced pressure from the resultant thus obtained, after treating the residue with a Na₂HCO₃ aqueous solution, the product was extracted therefrom using ethyl acetate and the combined organic extracts were dried using Na₂SO₄. The solvent was removed under reduced pressure from the obtained resultant, thereby obtaining Intermediate 1-c (yield: 80%).

LCMS: m/z calcd for C₁₂H₁₁BrN₂O 278.01; Found 279.02.

### Synthesis of Intermediate 1-d

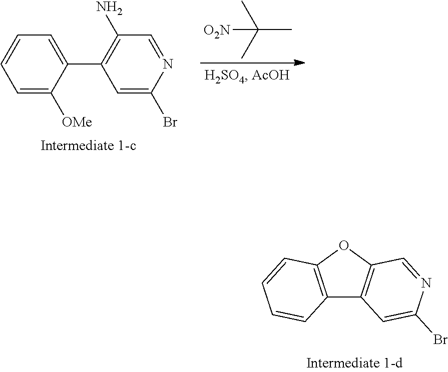

5.0 g (18.0 mmol) of Intermediate 1-c, 90 mL of glacial acetic acid, and 0.5 ml (18.1 mmol) of concentrated sulfuric acid were mixed together in a 500 mL of round-bottom flask, and 5.6 g (54.3 mmol) of 2-methyl-2-nitropropane were added dropwise thereto at ambient temperature. The mixture was then allowed to react at a temperature of 50° C. for 12 hours. Once the reaction was complete, the acetic acid was removed therefrom under reduced pressure, and the product was extracted therefrom using dichloromethane and a Na₂CO₃ aqueous solution. The combined organic extracts were treated with water, and dried using Na₂SO₄. The crude product was purified by silica gel column chromatography (using hexane and ethyl acetate at a ratio of 3:1), thereby obtaining Intermediate 1-d (yield: 45%).

LCMS: m/z calcd for C₁₁H₆BrNO 246.96; Found 248.0.

### Synthesis of Intermediate 1-e

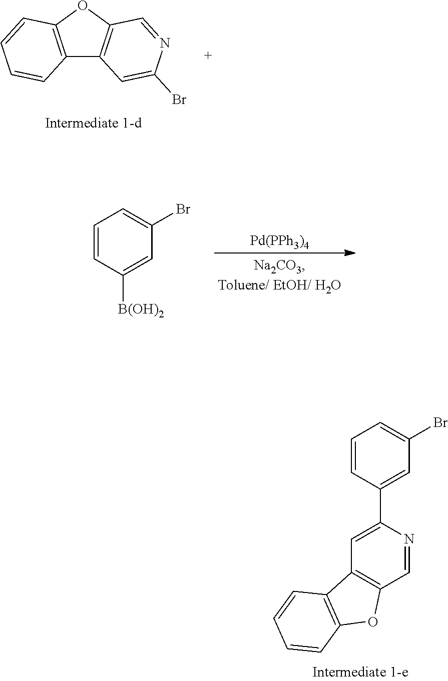 [SMILES: Brc1cc2c(cn1)oc1ccccc12 ; OB(O)c1cccc(Br)c1 ; Brc1cccc(-c2cc3c(cn2)oc2ccccc23)c1 ; "Pd(PPh3)4"]

10.4 g (42.0 mmol) of Intermediate 1-d, 12.65 g (63.0 mmol) of 2-bromophenyl-boronic acid, 3.4 g (2.94 mmol) of tetrakis(triphenylphosphine)palladium(0) (Pd(PPh₃)₄), and 11.1 g (105 mmol) of sodium carbonate (Na₂CO₃) were added to a flask. 90 mL of toluene, and 30 mL of ethanol and 30 mL of H₂O were added thereto, and the flask was purged with nitrogen gas. The mixture was allowed to react at a temperature of 105° C. for 24 hours. The resultant was cooled to room temperature, and the product was extracted therefrom using ethyl acetate and water. The combined organic extracts were dried using magnesium sulfate (MgSO₄). The resulting residue was then concentrated and purified by silica gel column chromatography (using hexane and ethyl acetate at a ratio of 7:1), thereby obtaining Intermediate 1-e (yield: 54%).

LCMS: m/z calcd for C₁₇H₁₀BrNO 322.99; Found 324.1.

### Synthesis of Intermediate 1-f

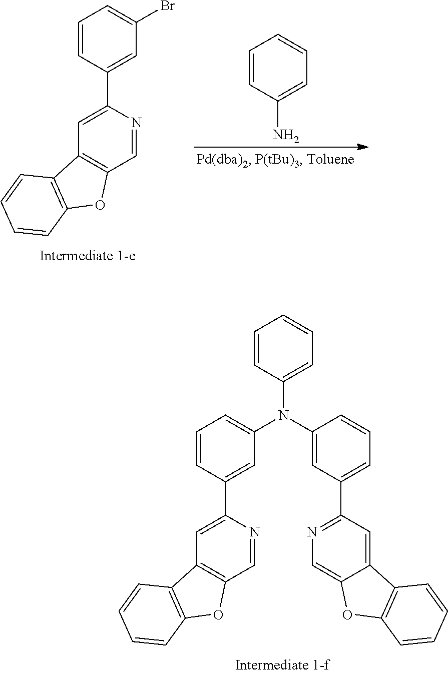 [SMILES: Brc1cccc(-c2cc3c(cn2)oc2ccccc23)c1 ; Nc1ccccc1 ; c1ccc(N(c2cccc(-c3cc4c(cn3)oc3ccccc34)c2)c2cccc(-c3cc4c(cn3)oc3ccccc34)c2)cc1]

4.8 g (15.0 mmol) of Intermediate 1-e, 1.0 g (6.0 mmol) of aniline, 0.34 g (0.6 mmol) of bis(dibenzylideneacetone)palladium(0) (Pd(dba)₂), 0.6 g (1.5 mmol) of tri-tert-butylphosphine (P(t-Bu)₃), and 1.73 g (18.0 mmol) of sodium tert-butoxide (NaOtBu) were added to a flask. Then, 50 mL of toluene was added to the mixture, the flask was purged with nitrogen gas, and the mixture was refluxed for 48 hours. The resultant was cooled to room temperature, and the product was extracted therefrom using dichloromethane and water, which was then dried using MgSO₄. The resulting residue was concentrated and purified by column chromatography (using hexane and dichloromethane at a ratio of 3:1), thereby obtaining Intermediate 1-f (yield: 65%).

LCMS: m/z calcd for C₄₀H₂₅N₃O₂ 579.19; Found 580.2.

### Synthesis of Compound 1

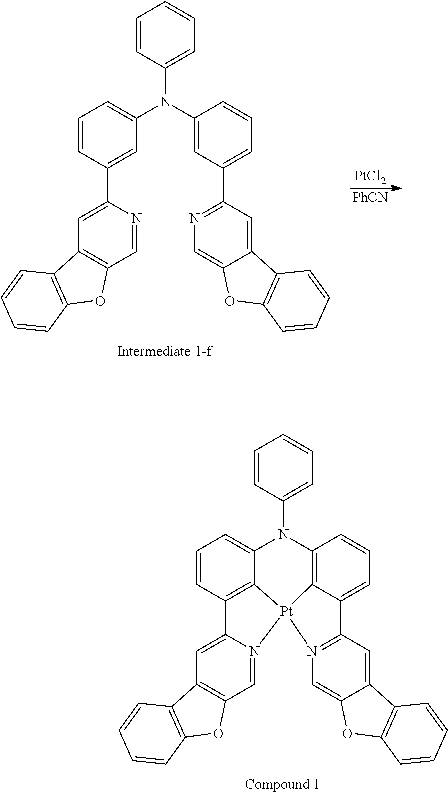

2.0 (3.5 mmol) of Intermediate 1-f, 0.93 g (3.5 mmol) of platinum chloride, and 100 mL of cyanobenzene were added to a 500 mL of round-bottom flask, and the mixture was refluxed for 24 hours. Once the reaction was complete, the resultant was cooled to room temperature, and a solvent was removed therefrom under reduced pressure. The resulting residue was purified by column chromatography using methylene chloride and normal hexane as eluents, thereby obtaining 1.2 g of Compound 1 (yield: 44%).

LCMS: m/z calcd for C₄₀H₂₃N₃O₂Pt 772.14; Found 773.2.

### Synthesis Example 2: Synthesis of Compound 2

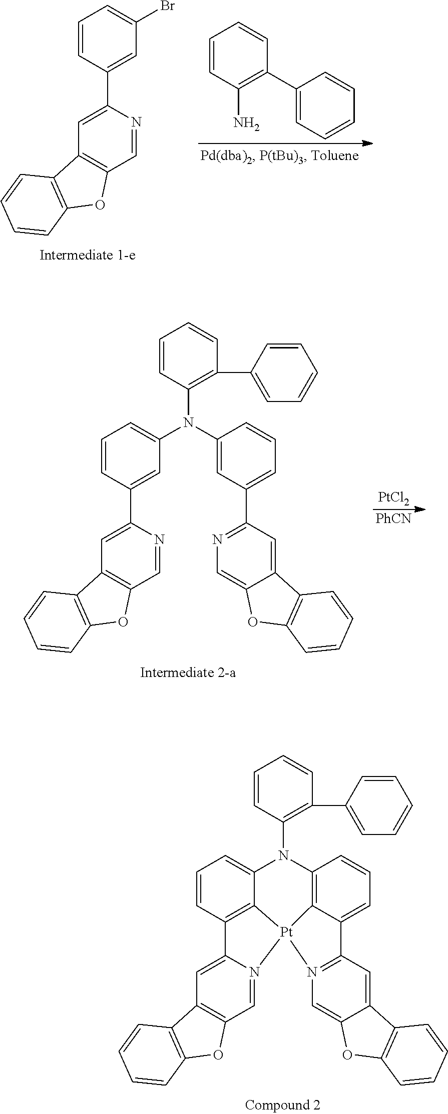

### Synthesis of Intermediate 2-a

Intermediate 2-a was synthesized in substantially the same manner as in Synthesis of Intermediate 1-f in Synthesis Example 1, except that 2-aminobiphenyl was used instead of aniline.

### Synthesis of Compound 2

Compound 2 was synthesized in substantially the same manner as in Synthesis of Compound 1 in Synthesis Example 1, except that Intermediate 2-a was used instead of Intermediate 1-f (yield: 54%).

LCMS: m/z calcd for C₄₆H₂₇N₃O₂Pt 848.18; Found 849.1

### Synthesis Example 3: Synthesis of Compound 3

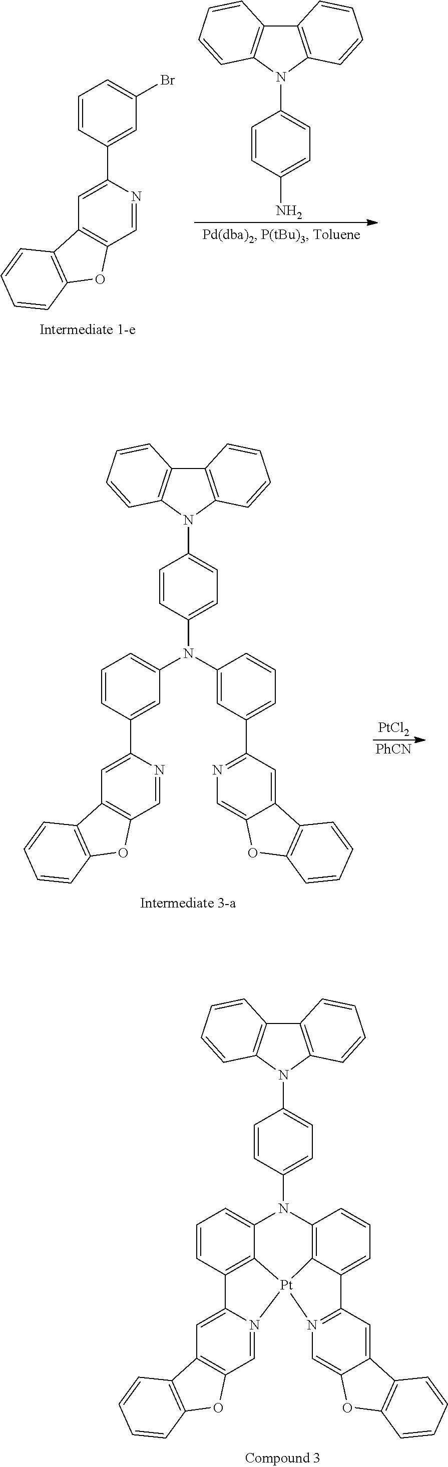

### Synthesis of Intermediate 3-a

Intermediate 3-a was synthesized in substantially the same manner as in Synthesis of Intermediate 1-f in Synthesis Example 1, except that 4-(9H-carbazol-9-yl)aniline was used instead of aniline.

### Synthesis of Compound 3

Compound 3 was synthesized in substantially the same manner as in Synthesis of Compound 1 in Synthesis Example 1, except that Intermediate 3-a was used instead of Intermediate 1-f (yield: 43%).

LCMS: m/z calcd for C₅₂H₃₀N₄O₂Pt 937.20; Found 938.2.

### Synthesis Example 4: Synthesis of Compound 4

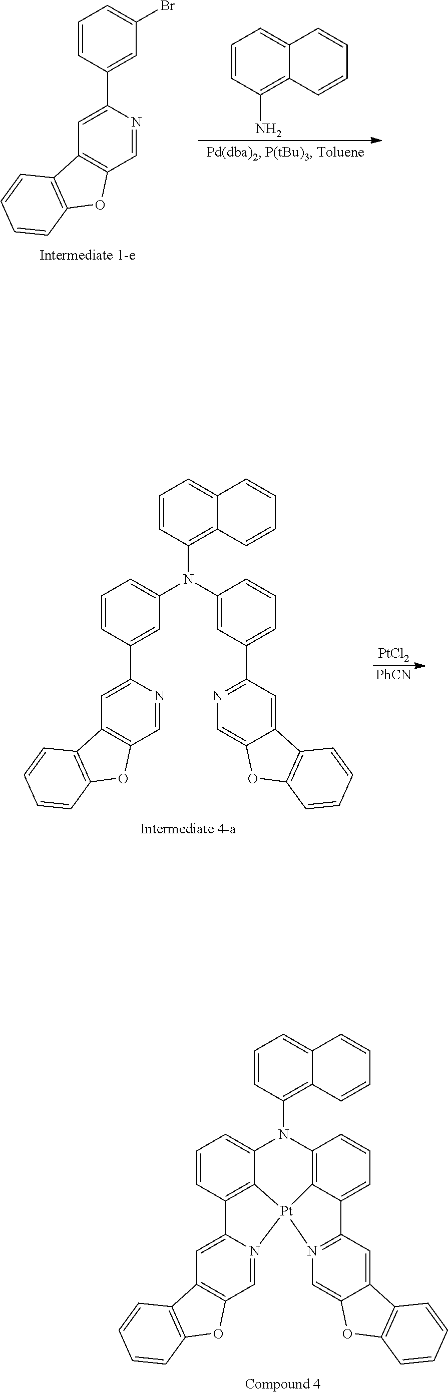

### Synthesis of Intermediate 4-a

Intermediate 4-a was synthesized in substantially the same manner as in Synthesis of Intermediate 1-f in Synthesis Example 1, except that naphthylaniline was used instead of aniline.

### Synthesis of Compound 4

Compound 4 was synthesized in substantially the same manner as in Synthesis of Compound 1 in Synthesis Example 1, except that Intermediate 4-a was used instead of Intermediate 1-f (yield: 35%).

LCMS: m/z calcd for C₄₄H₂₅N₃O₂Pt 822.16; Found 823.2.

### Synthesis Example 5: Synthesis of Compound 5

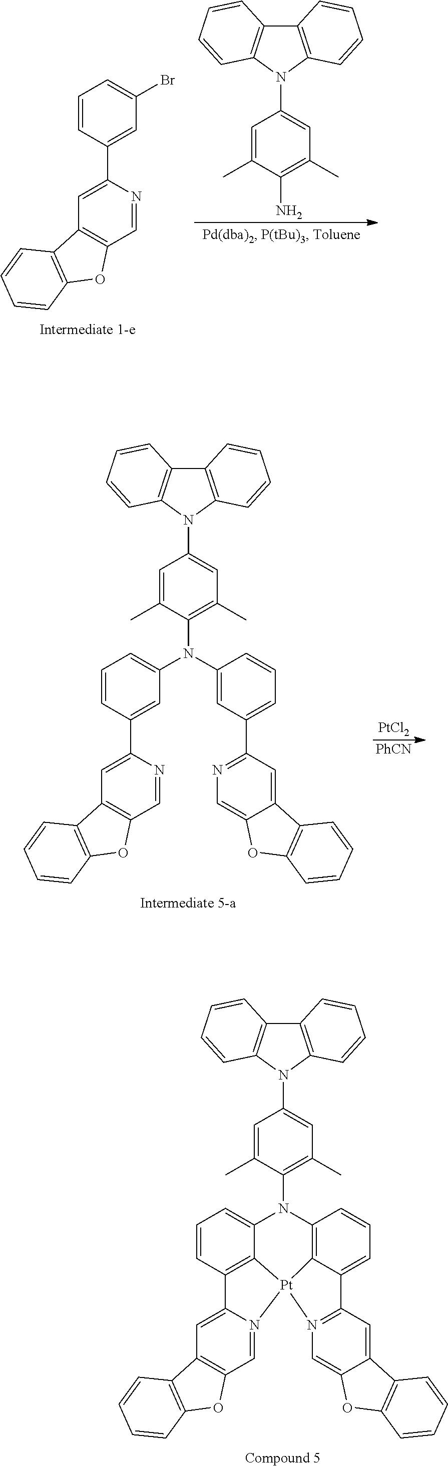

### Synthesis of Intermediate 5-a

Intermediate 5-a was synthesized in substantially the same manner as in Synthesis of Intermediate 1-f in Synthesis Example 1, except that 4-(9H-carbazol-9-yl)-2,6-dimethylaniline was used instead of aniline.

### Synthesis of Compound 5

Compound 5 was synthesized in substantially the same manner as in Synthesis of Compound 1 in Synthesis Example 1, except that Intermediate 5-a was used instead of Intermediate 1-f (yield: 35%).

LCMS: m/z calcd for C₅₄H₃₄N₄O₂Pt 965.23; Found 966.2.

Evaluation Example 1: Evaluation of Ultraviolet (UV)-Visible (Vis) Absorption Spectrum and Photoluminance (PL) Spectrum

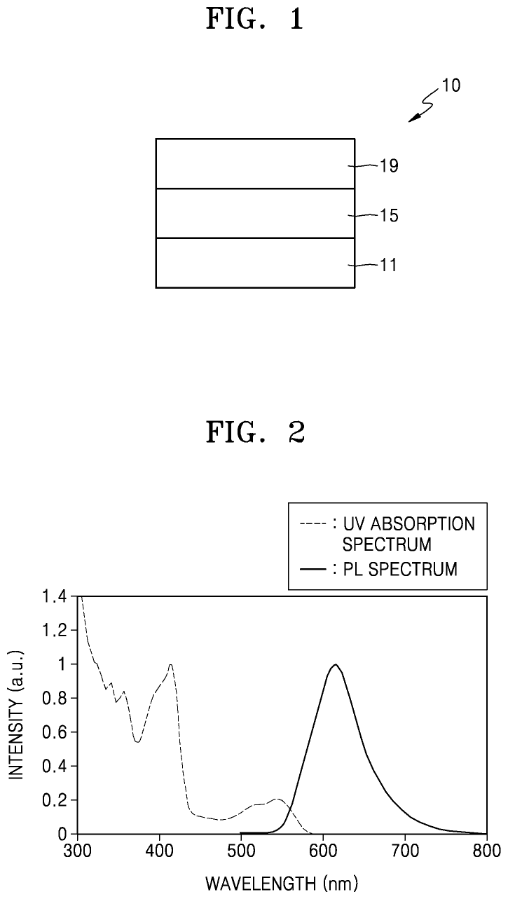

Compound 5 was diluted in 2-methyltetrahydrofuran at a concentration of 1×10⁻⁵ molar (M), and a UV-Vis absorption spectrum thereof was measured using a Shimadzu UV-350 Spectrometer. Compound 5 was diluted in 2-methyltetrahydrofuran at a concentration of 10 millimolar (mM), and a PL spectrum thereof was measured at room temperature using an ISC PC1 spectrofluorometer, in which a Xenon lamp was mounted. The results thereof are shown in FIG. 2. As shown in FIG. 2, Compound 5 was found to have a suitable UV-Vis absorption spectrum and a suitable PL spectrum for use in an organic light-emitting device.

### Example 1

A glass substrate, on which an anode having an ITO/Ag/ITO (70 Å/1,000 Å/70 Å) structure was deposited, was cut to a size of 50 millimeters (mm)×50 mm×0.5 mm, sonicated in iso-propyl alcohol and water for 5 minutes each, and cleaned by exposure to ultraviolet rays and ozone for 30 minutes. Subsequently, the glass substrate was mounted on a vacuum-deposition device.

2-TNATA was deposited on the anode to form a hole injection layer having a thickness of 600 Å. 4,4′-bis[N-(1-naphthyl)-N-phenylamino]biphenyl (hereinafter, referred as “NPB”) was deposited on the hole injection layer to form a hole transport layer having a thickness of 1,350 Å.

CBP (as a host) and Compound 1 (as a dopant) were co-deposited on the hole transport layer at a weight ratio of 94:6 to form an emission layer having a thickness of 400 Å. BCP was deposited on the emission layer to form a hole blocking layer having a thickness of 50 Å. Subsequently, Alq₃ was deposited on the hole blocking layer to form an electron transport layer having a thickness of 350 Å. LiF was deposited on the electron transport layer to form an electron injection layer having a thickness of 10 Å. MgAg was deposited on the electron injection layer at a weight ratio of 90:10 to form a cathode having a thickness of 120 Å, thereby completing the manufacture of an organic light-emitting device (which emits red light).

### Examples 2 to 5 and Comparative Examples A to C

Organic light-emitting devices were manufactured in substantially the same manner as in Example 1, except that the compounds shown in Table 2 were used instead of Compound 1 as a dopant in the formation of an emission layer.

Evaluation Example 2: Evaluation of Characteristics of Organic Light-Emitting Device

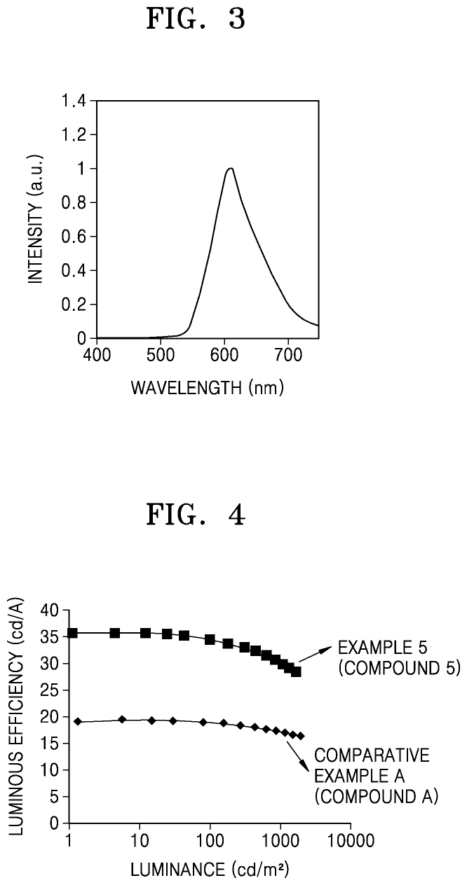

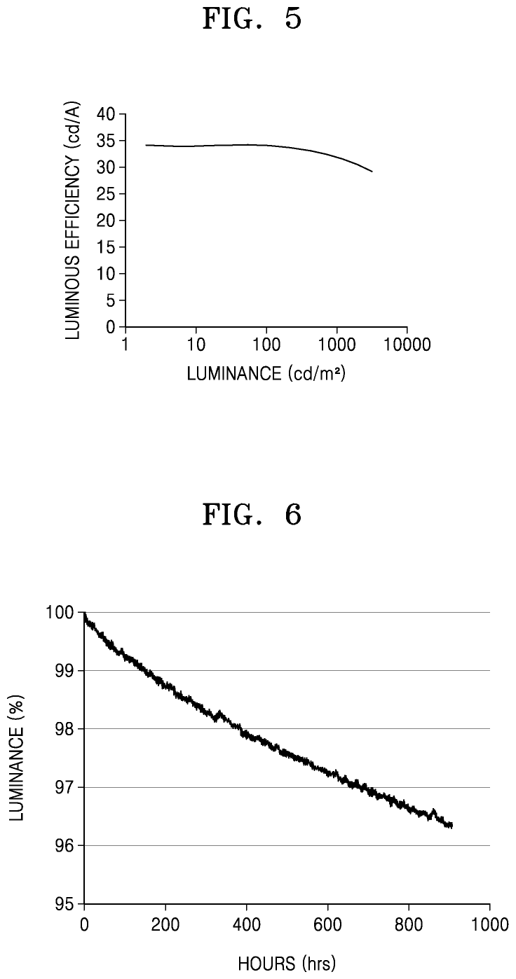

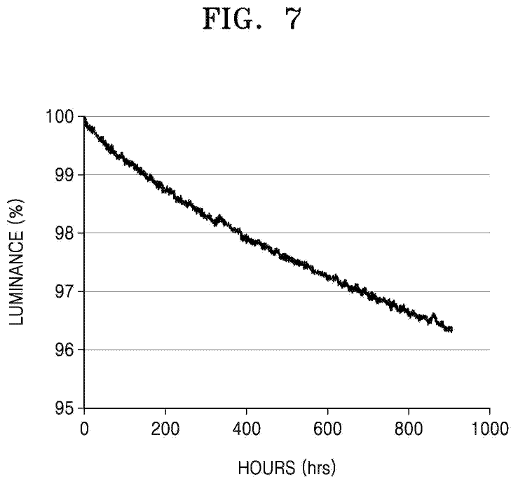

A Keithley 2400 current voltmeter and a luminance meter (Minolta Cs-1000A) were used to evaluate the driving voltage, luminous efficiency, quantum emission efficiency, maximum emission wavelength, full width at half maximum (FWHM), and color-coordinate of the organic light-emitting devices manufactured in Examples 1 to 5 and Comparative Examples A to C. The evaluation results are shown in Table 2, FIG. 3 (which illustrates an electroluminance (EL) spectrum of the organic light-emitting device of Example 5), FIG. 4 (which illustrates a luminance vs. luminous efficiency graph of the organic light-emitting devices of Example 5 and Comparative Example A), FIG. 5 (which illustrates a luminance vs. luminous efficiency graph of the organic light-emitting device of Example 2), and FIG. 6 (which illustrates a time vs. luminance graph of the organic light-emitting device of Example 2).

A quartz substrate washed with chloroform and pure water was prepared. Compound 1 was vacuum-deposited on the quartz substrate at a vacuum degree of 10⁻⁷ torr to prepare a film having a thickness of 50 nm. This film was evaluated at room temperature by using a TRPL measurement system, FluoTime 300 (available from PicoQuant), and a pumping source, PLS340 (available from PicoQuant, excitation wavelength=340 nm, spectral width=20 nm). Then, a wavelength of the main peak in the PL spectrum was determined, and upon photon pulses (pulse width=500 picoseconds, ps) applied to the film by PLS340, the number of photons emitted at a wavelength of the main peak for the film was repeatedly measured over time by time-correlated single photon counting (TCSPC), thereby obtaining TRPL curves available for the sufficient fitting. Based on the results obtained therefrom, two or more exponential decay functions were set forth for the fitting, thereby obtaining a decay time for Compound 1. Thiiridiums process was performed on Compounds 2 to 5 and Compounds A to C. The results thereof are also shown in Table 2.

TABLE 2

| | | | | Quantum | Maximum | | | |
|---|---|---|---|---|---|---|---|---|
| | Dopant | Driving | Luminous | emission | emission | | Decay | |
| | Compound | voltage | efficiency | efficiency | wavelength | FWHM | time | |
| | No. | (V) | (cd/A) | (%) | (nm) | (nm) | (μs) | CIEx |
| Example 1 | 1 | 4.45 | 25.5 | 18.5 | 613 | 81.8 | 4.02 | 0.608 |
| Example 2 | 2 | 4.42 | 24.1 | 16.6 | 616 | 80.4 | 3.50 | 0.617 |
| Example 3 | 3 | 4.32 | 28.9 | 17.0 | 611 | 83.0 | 3.50 | 0.605 |
| Example 4 | 4 | 4.35 | 26.8 | 17.2 | 613 | 82.5 | 3.58 | 0.613 |
| Example 5 | 5 | 4.30 | 29.1 | 17.7 | 613 | 82.9 | 3.52 | 0.617 |
| Comparative | A | 4.51 | 16.1 | 15.2 | 615 | 68.2 | 6.98 | 0.622 |
| Example A | | | | | | | | |
| Comparative | B | 4.97 | 18.3 | 12.6 | 609 | 78.2 | 7.23 | 0.612 |
| Example B | | | | | | | | |
| Comparative | C | 4.87 | 21.0 | 15.8 | 611 | 74.3 | 6.63 | 0.638 |
| Example C | | | | | | | | |

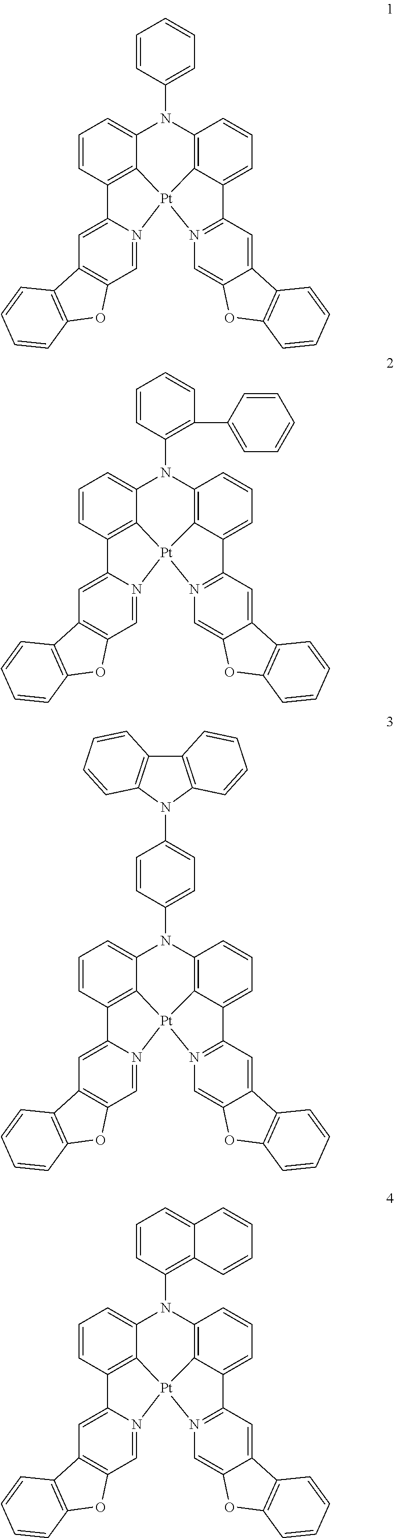

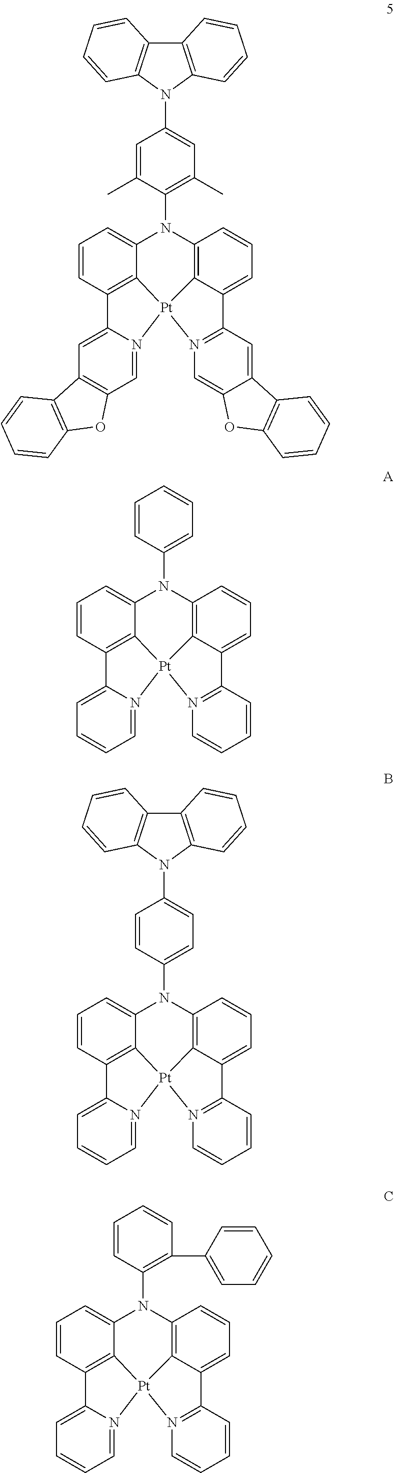

Referring to Table 2 and FIGS. 3 to 6, the organic light-emitting device of Examples 1 to 5 were found to have excellent driving voltage, luminous efficiency, quantum emission efficiency, and decay time, as compared with the organic light-emitting device of Comparative Examples A to C.

As apparent from the foregoing description, the organometallic compound has excellent electrical characteristics and thermal stability. Accordingly, an organic light-emitting device employing the organometallic compound has a low driving voltage, high efficiency, high power, high color purity, and excellent lifespan characteristics. Further, a diagnostic composition that includes the organometallic compound may have high diagnostic efficiency, because the organometallic compound is excellent in phosphorescent emission characteristics.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present disclosure as defined by the following claims.

What is claimed is:

1. An organometallic compound represented by Formula 1(1): 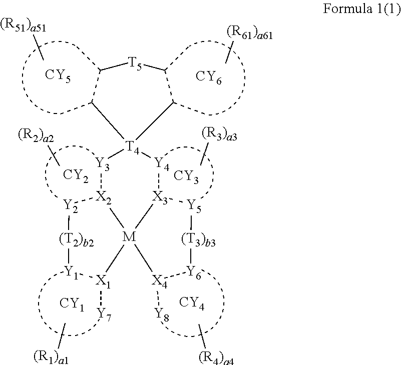
wherein, in Formula 1(1),
M is selected from platinum (Pt) and palladium (Pd),
X₁ is nitrogen (N),
X₂ to X₄ are each independently selected from carbon (C) and N,
a bond between X₁ and M is a coordinate bond; and one bond selected from a bond between X₂ and M, a bond between X₃ and M, and a bond between X₄ and M is a coordinate bond, while the other two bonds are each a covalent bond,
Y₁ to Y₆ are each independently selected from C and N,
Y₇ and Y₈ are each independently selected from C, N, oxygen (O), and sulfur (S),
a bond between X₁ and Y₇, a bond between X₁ and Y₁, a bond between X₂ and Y₂, a bond between X₂ and Y₃, a bond between X₃ and Y₄, a bond between X₃ and Y₅, a bond between X₄ and Y₆, and a bond between X₄ and Y₈ are each independently selected from a single bond and a double bond,
CY₁ is selected from an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene-5-oxide group, an aza-9H-fluoren-9-one group, and an azadibenzothiophene-5,5-dioxide group, each comprising at least one N as a ring-forming atom,
CY₂ to CY₄ are each independently selected from a C₅-C₃₀ carbocyclic group and a C₁-C₃₀ heterocyclic group,
CY₅ and CY₆ are each independently selected from a C₅-C₃₀ carbocyclic group and a C₁-C₃₀ heterocyclic group,
T₂ and T₃ are each independently selected from *—N[(L₅)ₐ₅-(R₅)]—*′, *—B(R₅)—*′, *—P(R₅)—*′, *—C(R₅)(R₆)—*′, *—Si(R₅)(R₆)—*′, *—Ge(R₅)(R₆)—*′, *—S—*′, *—Se—*′, *—O—*′, *—C(═O)—*′, *—S(═O)—*′, *—S(═O)₂—*, *—C(R₅)═*′, *═C(R₅)—*, *—C(R₅)═C(R₆)—*, *—C(═S)—*′, and *—C≡C—*′,
T₄ is selected from C, silicon (Si), and germanium (Ge),
T₅ is a single bond,
L₅ is selected from a single bond, a substituted or unsubstituted C₅-C₃₀ carbocyclic group, and a substituted or unsubstituted C₁-C₃₀ heterocyclic group,
a5 is an integer from 1 to 3; when a5 is 2 or greater, at least two L₅ groups are identical to or different from each other,
R₅ and R₆ are optionally bound via a first linking group to form a substituted or unsubstituted C₅-C₃₀ carbocyclic group or a substituted or unsubstituted C₁-C₃₀ heterocyclic group,
b2 and b3 are each independently selected from 0, 1, 2, and 3; provided that when b2 is 0, *-(T₂)_{b2}-*′ is a single bond; and when b3 is 0, *-(T₃)_{b3}-*′ is a single bond,
R₁ to R₆, R₅₁ and R₆₁ are each independently selected from hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), and —P(═O)(Q₈)(Q₉),
a1 to a4 are each independently selected from 0, 1, 2, 3, 4, and 5,
a51 and a61 are each independently selected from 0, 1, 2, and 3,
two of R₁ groups in the number of a1 are optionally bound to form a substituted or unsubstituted C₅-C₃₀ carbocyclic group or a substituted or unsubstituted C₁-C₃₀ heterocyclic group,
two of R₂ groups in the number of a2 are optionally bound to form a substituted or unsubstituted C₅-C₃₀ carbocyclic group or a substituted or unsubstituted C₁-C₃₀ heterocyclic group,
two of R₃ groups in the number of a3 are optionally bound to form a substituted or unsubstituted C₅-C₃₀ carbocyclic group or a substituted or unsubstituted C₁-C₃₀ heterocyclic group,
two of R₄ groups in the number of a4 are optionally bound to form a substituted or unsubstituted C₅-C₃₀ carbocyclic group or a substituted or unsubstituted C₁-C₃₀ heterocyclic group,
at least two of R₁ to R₄, wherein the at least two of R₁ to R₄ are adjacent to each other, are optionally bound to form a substituted or unsubstituted C₅-C₃₀ carbocyclic group or a substituted or unsubstituted C₁-C₃₀ heterocyclic group,
at least one of R₅ and R₆ and at least one of R₁ to R₄ are optionally bound to form a substituted or unsubstituted C₅-C₃₀ carbocyclic group or a substituted or unsubstituted C₁-C₃₀ heterocyclic group,
at least one substituent of the substituted C₅-C₃₀ carbocyclic group, substituted C₁-C₃₀ heterocyclic group, substituted C₁-C₆₀ alkyl group, substituted C₂-C₆₀ alkenyl group, substituted C₂-C₆₀ alkynyl group, substituted C₁-C₆₀ alkoxy group, substituted C₃-C₁₀ cycloalkyl group, substituted C₁-C₁₀ heterocycloalkyl group, substituted C₃-C₁₀ cycloalkenyl group, substituted C₁-C₁₀ heterocycloalkenyl group, substituted C₆-C₆₀ aryl group, substituted C₆-C₆₀ aryloxy group, substituted C₆-C₆₀ arylthio group, substituted C₁-C₆₀ heteroaryl group, substituted monovalent non-aromatic condensed polycyclic group, and substituted monovalent non-aromatic condensed heteropolycyclic group is selected from:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), and —P(═O)(Q₁₈)(Q₁₉);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉); and
—N(Q₃₁)(Q₃₂), —Si(Q₃₃)(Q₃₄)(Q₃₅), —B(Q₃₆)(Q₃₇), and —P(═O)(Q₃₈)(Q₃₉),
wherein Q₁ to Q₉, Q₁₁ to Q₁₉, Q₂₁ to Q₂₉, and Q₃₁ to Q₃₉ are each independently selected from a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ alkyl group, and a C₆-C₆₀ aryl group.

2. The organometallic compound of claim 1, wherein
X₂ and X₃ are each C, X₄ is N,
a bond between X₂ and M and a bond between X₃ and M are each a covalent bond, and
a bond between X₄ and M is a coordinate bond.

3. The organometallic compound of claim 1, wherein CY₂ to CY₄ are each independently selected from a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indene group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene-5,5-dioxide group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene-5-oxide group, an aza-9H-fluoren-9-one group, an azadibenzothiophene-5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, and a 5,6,7,8-tetrahydroquinoline group.

4. The organometallic compound of claim 1, wherein CY₁ is identical to CY₄.

5. The organometallic compound of claim 1, wherein b2 and b3 are each independently selected from 0 and 1.

6. The organometallic compound of claim 1, wherein R₁ to R₆, R₅₁ and R₆₁ are each independently selected from:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, and a C₁-C₂₀ alkoxy group;
a C₁-C₂₀ alkyl group and a C₁-C₂₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, and a pyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, and an imidazopyrimidinyl group; and
—N(Q₁)(Q₂), —Si(Q₃)(Q₄)(Q₅), —B(Q₆)(Q₇), and —P(═O)(Q₈)(Q₉),
wherein Q₁ to Q₉ are each independently selected from:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, and —CD₂CDH₂;
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group; and
an n-propyl group, an iso-propyl group, an n-butyl group, an iso-butyl group, a sec-butyl group, a tert-butyl group, an n-pentyl group, an iso-pentyl group, a sec-pentyl group, a tert-pentyl group, a phenyl group, and a naphthyl group, each substituted with at least one selected from deuterium, a C₁-C₁₀ alkyl group, and a phenyl group.

7. The organometallic compound of claim 1, wherein R₁ to R₆, R₅₁ and R₆₁ are each independently selected from hydrogen, deuterium, —F, a cyano group, a nitro group, —SF₅, —CH₃, —CD₃, —CD₂H, —CDH₂, —CF₃, —CF₂H, —CFH₂, groups represented by Formulae 9-1 to 9-20, groups represented by Formulae 10-1 to 10-152, and —Si(Q₃)(Q₄)(Q₅): 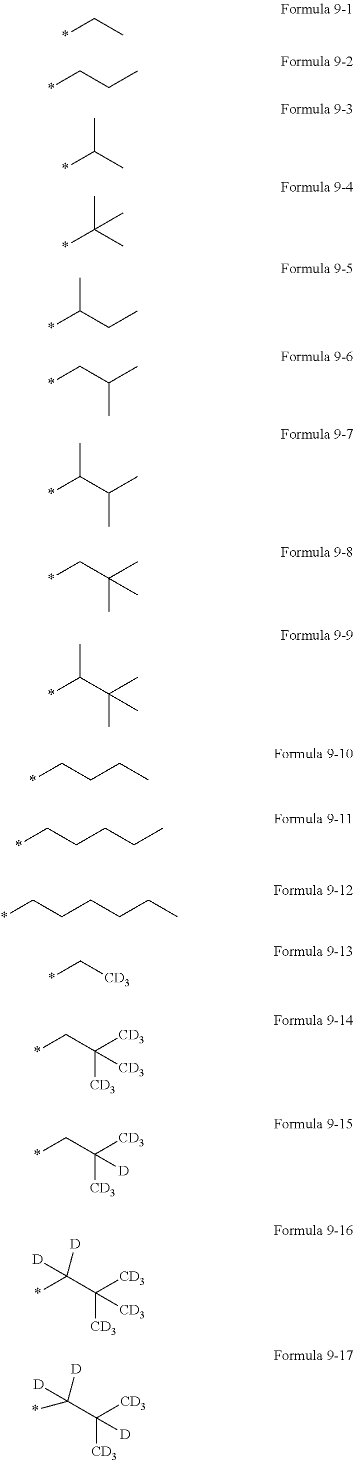 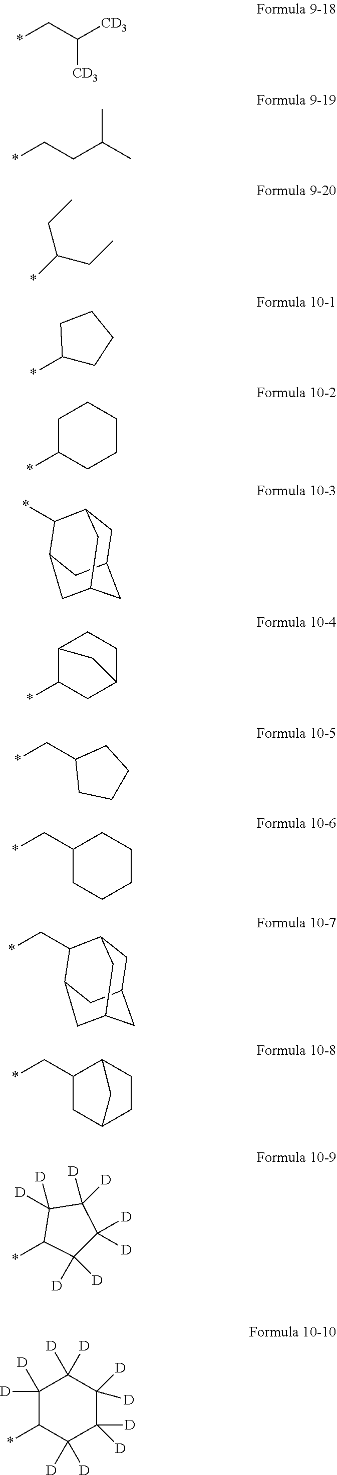 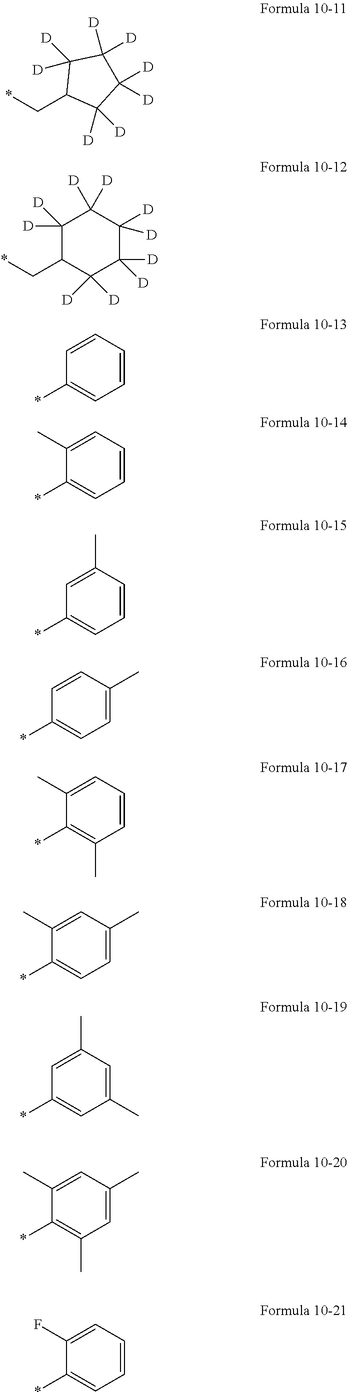 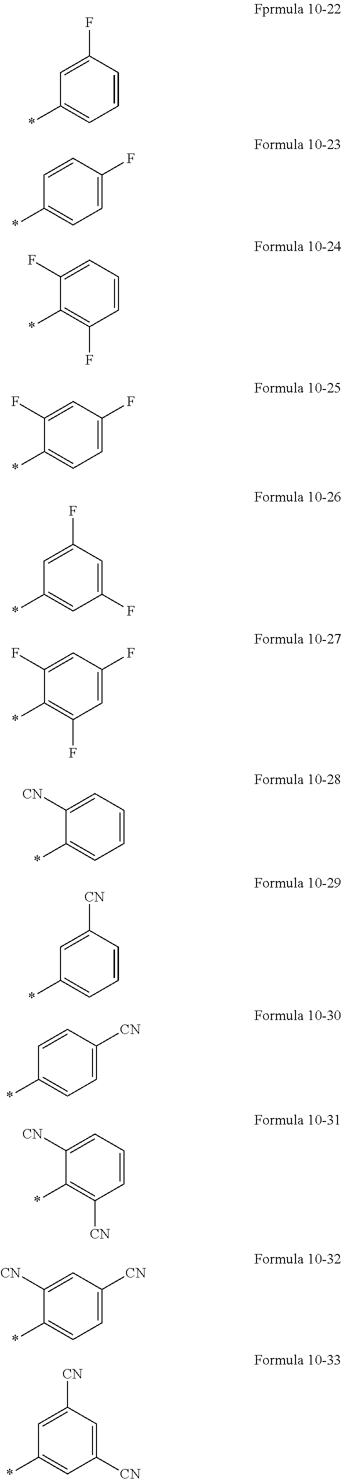 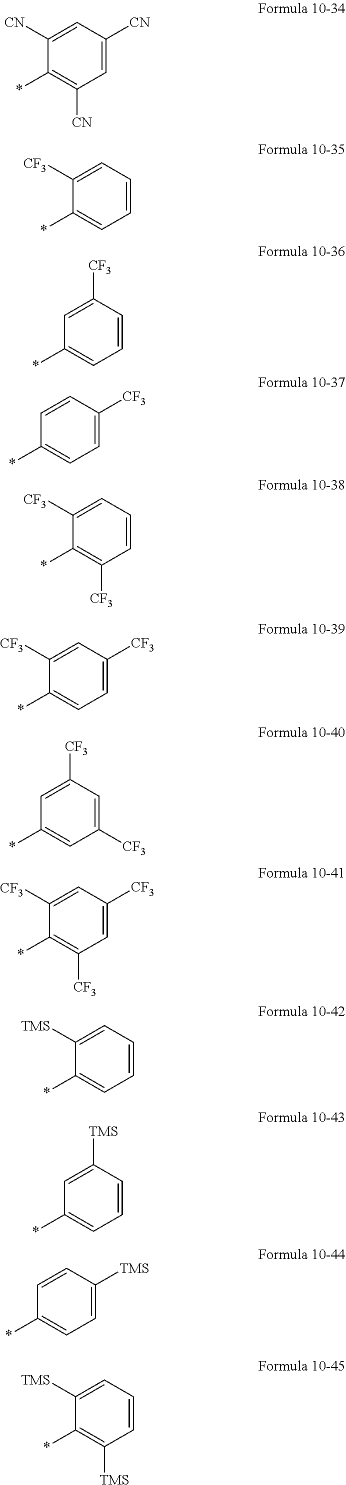 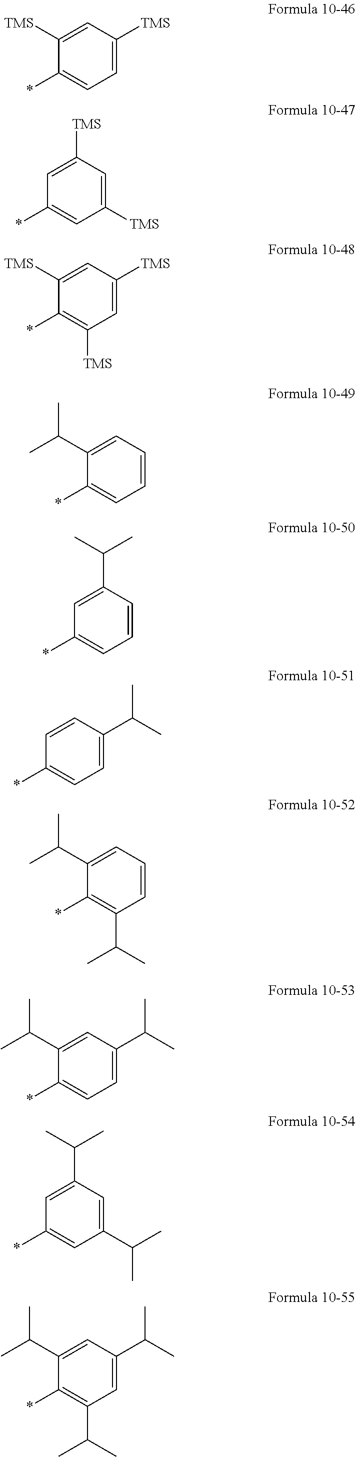 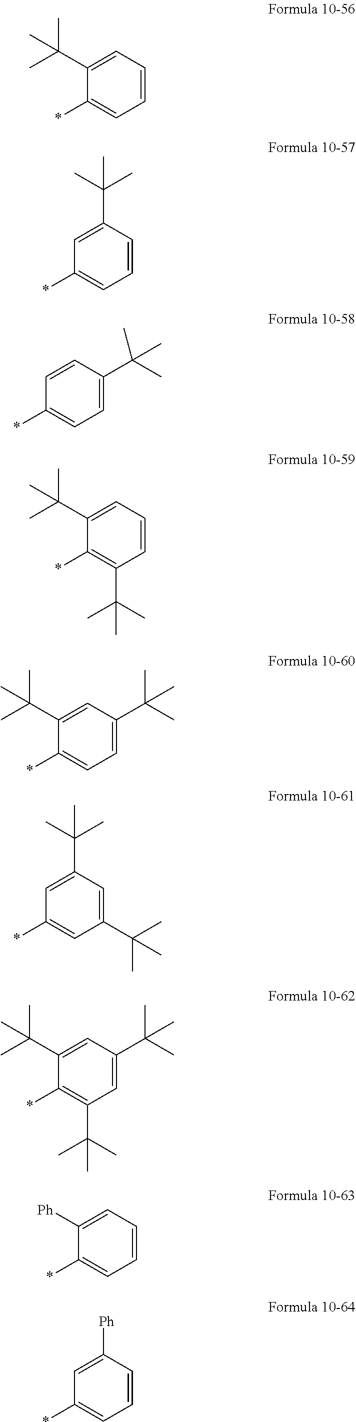 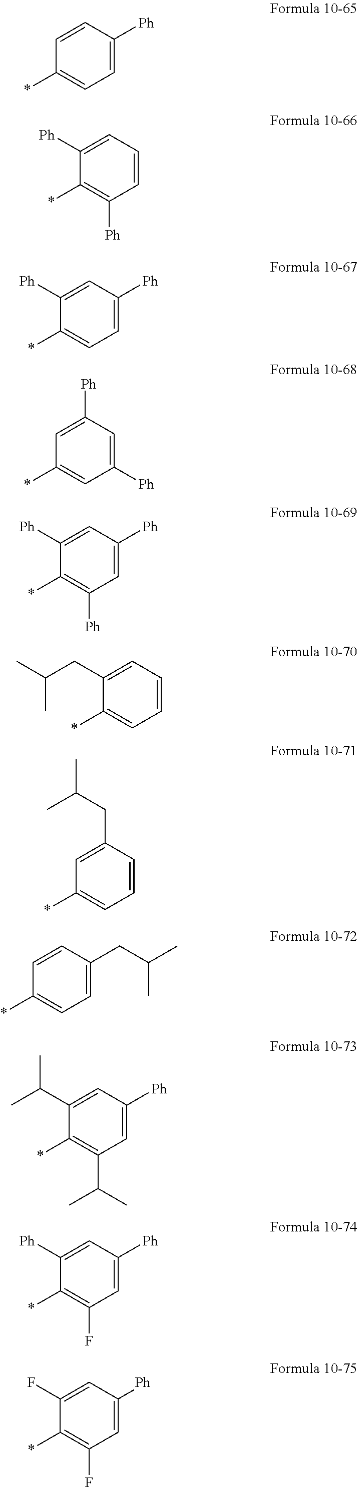 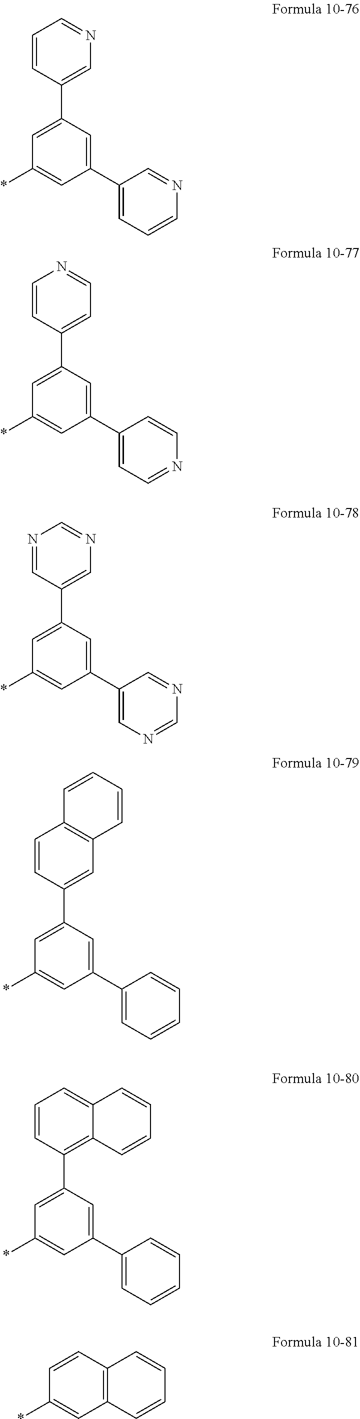 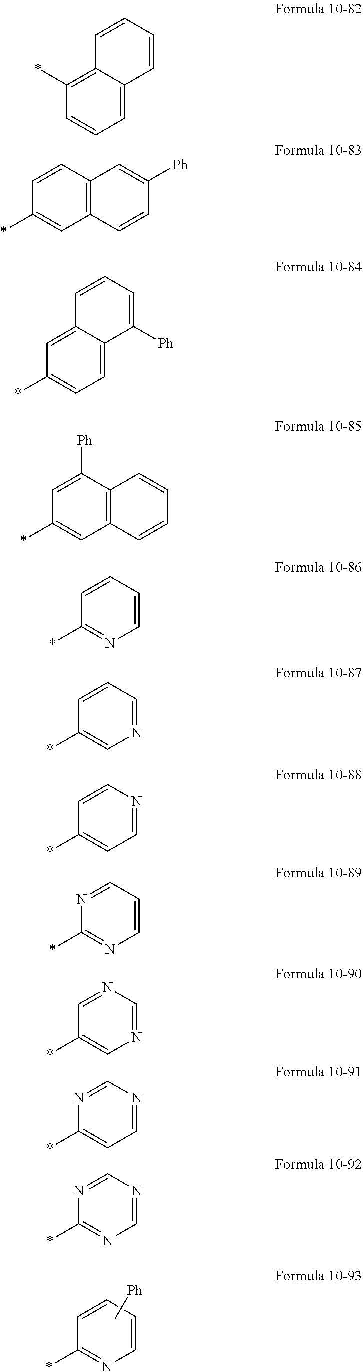 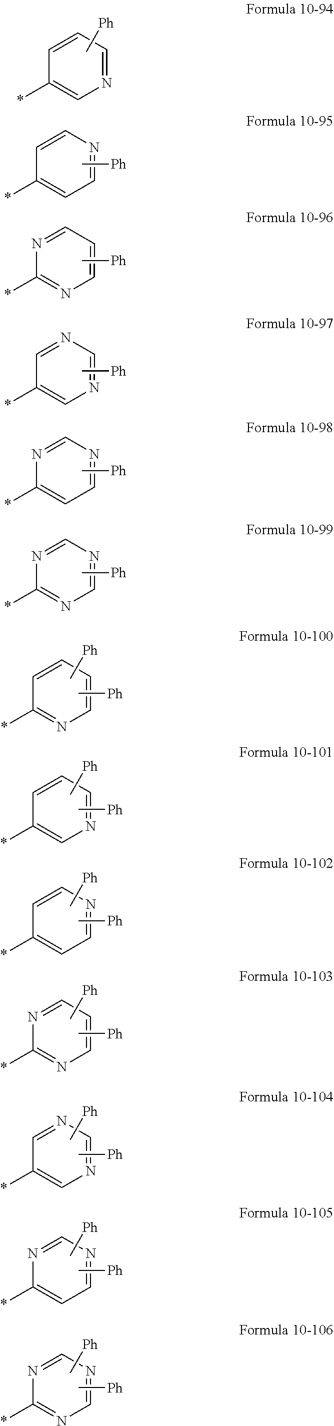 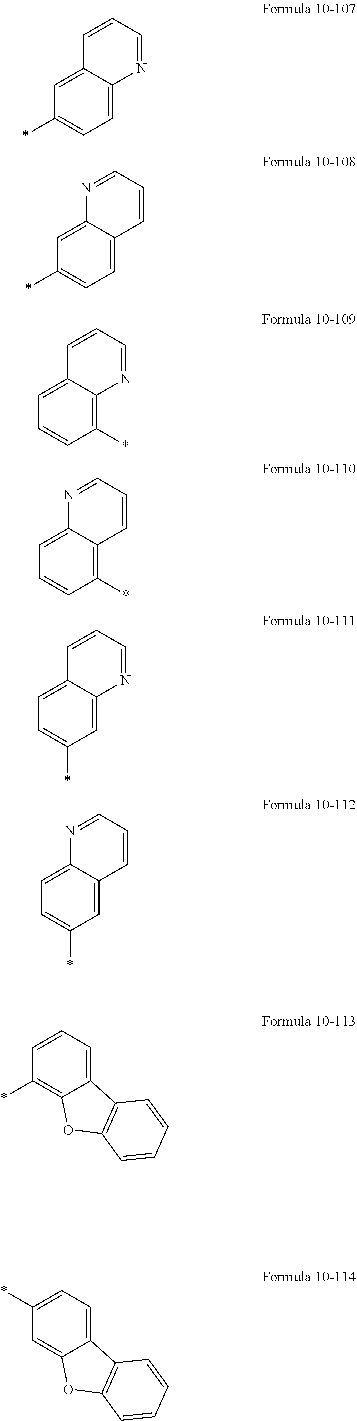 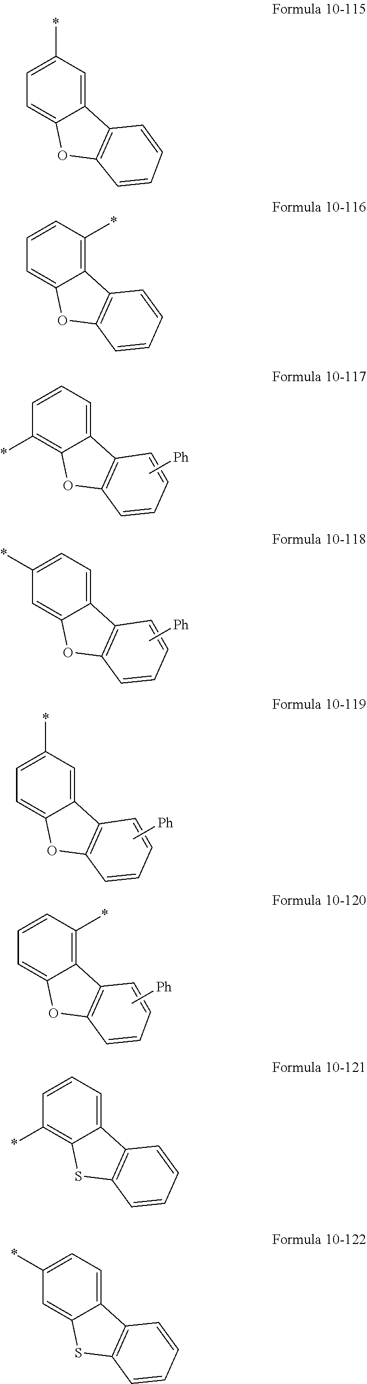 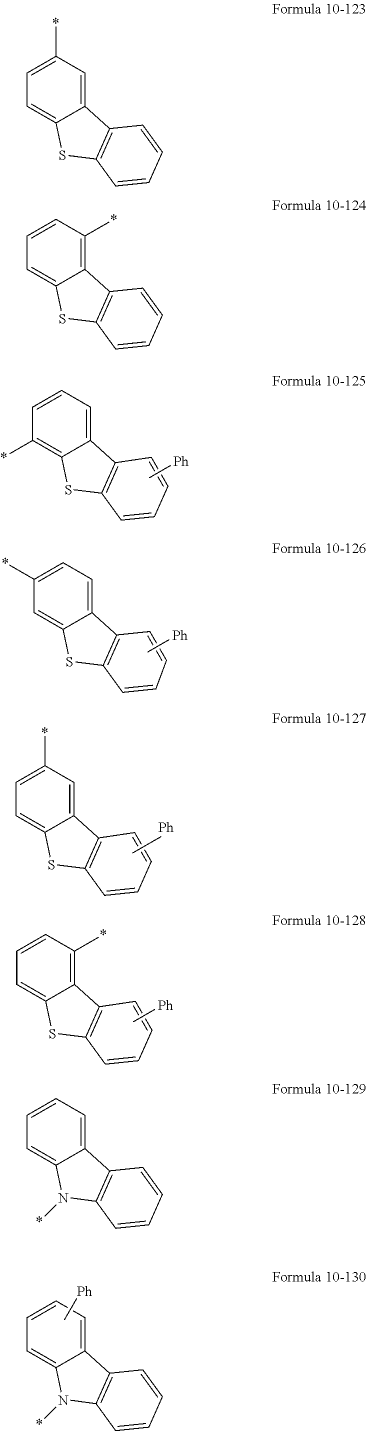 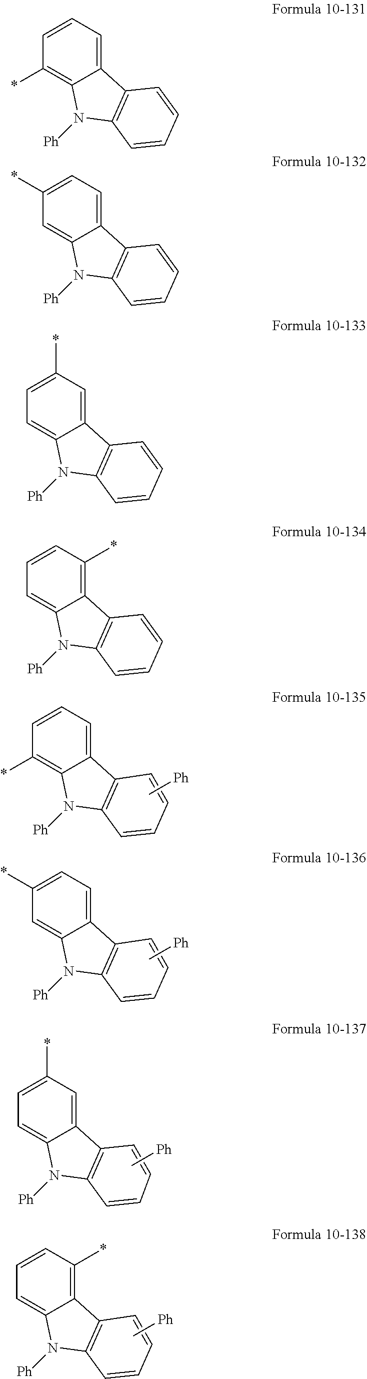 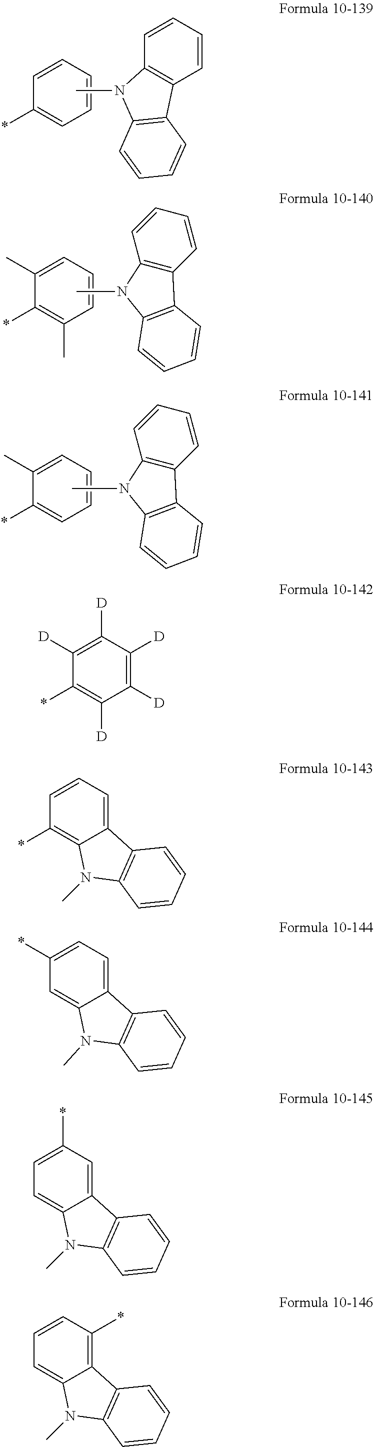 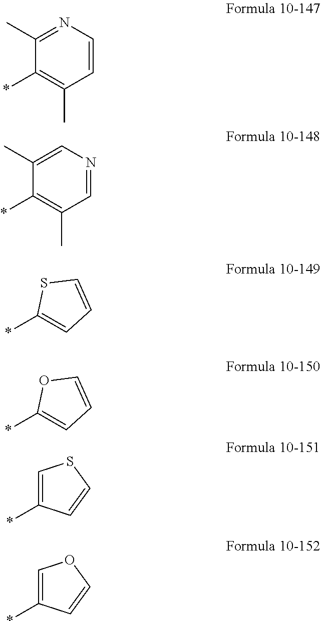
wherein, in Formulae 9-1 to 9-20 and 10-1 to 10-152, * indicates a binding site to an adjacent atom, “Ph” represents a phenyl group, and “TMS” represents a trimethylsilyl group.

8. The organometallic compound of claim 1, wherein a moiety represented by 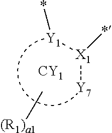
in Formula 1 is selected from groups represented by Formulae CY1-1 to CY1-6: 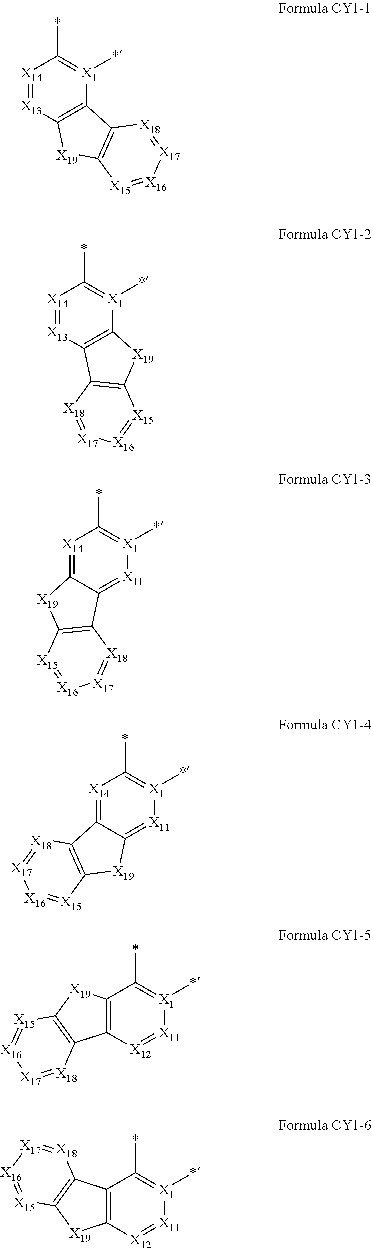
wherein, in Formulae CY1-1 to CY1-6,
X₁ is N,
X₁₁ is N or C(R₁₁), X₁₂ is N or C(R₁₂), X₁₃ is N or C(R₁₃), X₁₄ is N or C(R₁₄), X₁₅ is N or C(R₁₅), X₁₆ is N or C(R₁₆), X₁₇ is N or C(R₁₇), X₁₅ is N or C(R₁),
X₁₉ is selected from *—N[(L₁₉)ₐ₁₉-(R₁₉)]—*′, *—B(R₁₉)—*′, *—P(R₁₉)—*′, *—C(R₁₉ₐ)(R_{19b})—*, *—Si(R₁₉ₐ)(R_{19b})—*′, *—Ge(R₁₉ₐ)(R_{19b})—*′, *—S—*′, *—Se—*′, *—O—*′, *—C(═O)—*′, *—S(═O)—*′, *—S(═O)₂—*′, and *—C(═S)—*′,
L₁₉ and a19 are each the same as described in claim 1 with reference to L₅ and a5, respectively,
R₁₁ to R₁₉, R₁₉ₐ, and R_{19b} are each independently the same as described in claim 1 with reference to R₁, and
* and *′ each indicate a binding site to an adjacent atom.

9. The organometallic compound of claim 1, wherein a moiety represented by 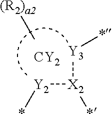
in Formula 1 is selected from groups represented by Formulae CY2-1 to CY2-21: 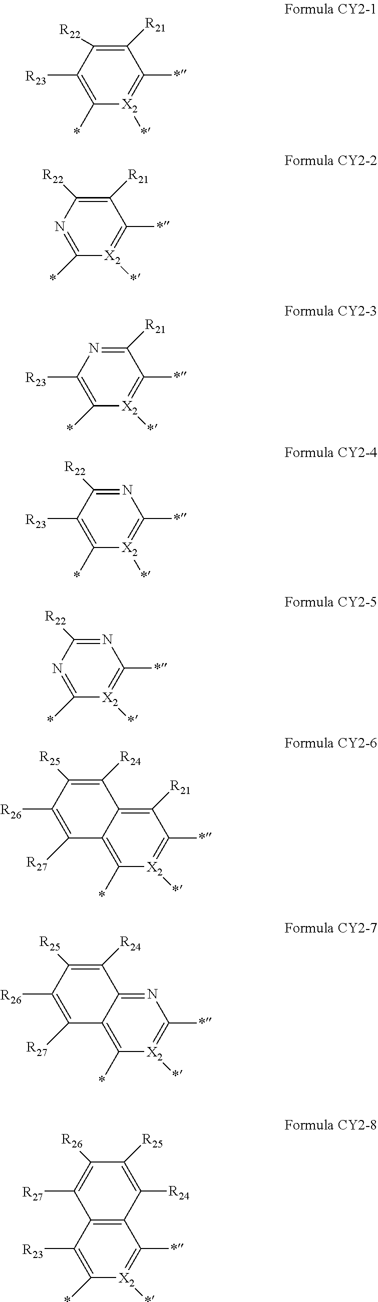 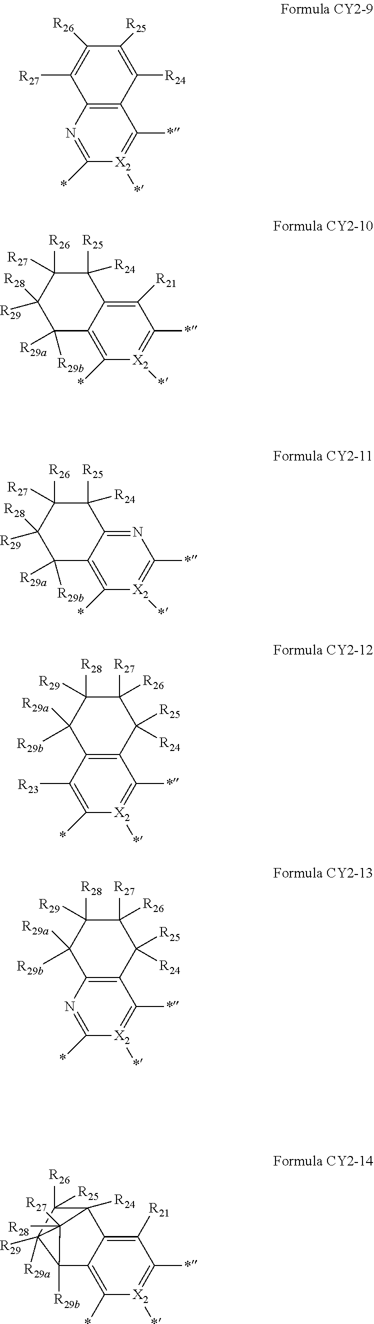 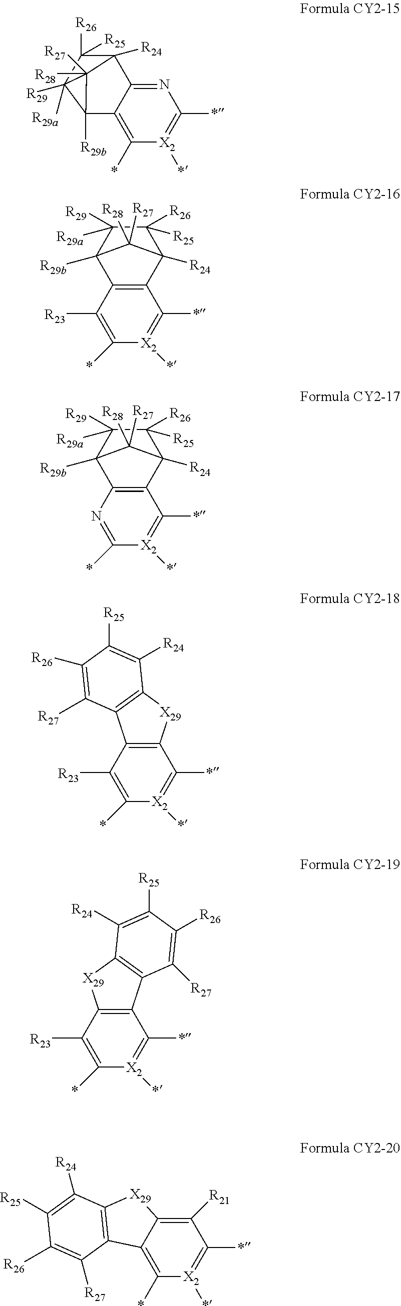 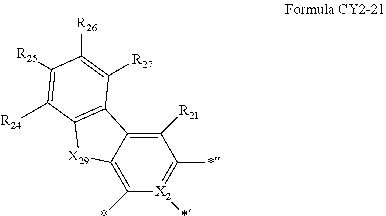
wherein, in Formulae CY2-1 to CY2-21,
X₂ is selected from C and N,
X₂₉ is selected from *—N[(L₂₉)ₐ₂₉-(R₂₉)]—*′, *—B(R₂₉)—*′, *—P(R₂₉)—*′, *—C(R₂₉ₐ)(R_{29b})—*′, *—Si(R₂₉ₐ)(R_{29b})—*′, *—Ge(R₂₉ₐ)(R_{29b})—*′, *—S—*′, *—Se—*′, *—O—*′, *—C(═O)—*′, *—S(═O)—*′, *—S(═O)₂—*′, and *—C(═S)—*′,
L₂₉ and a29 are each the same as described in claim 1 with reference to L₅ and a5, respectively,
R₂₁ to R₂₉, R₂₉ₐ, and R_{29b} are each independently the same as described in claim 2 with reference to R₁, and
*, *′, and *″ each indicate a binding site to an adjacent atom.

10. The organometallic compound of claim 1, wherein a moiety represented by 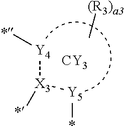
in Formula 1 is selected from groups represented by Formulae CY3-1 to CY3-21: 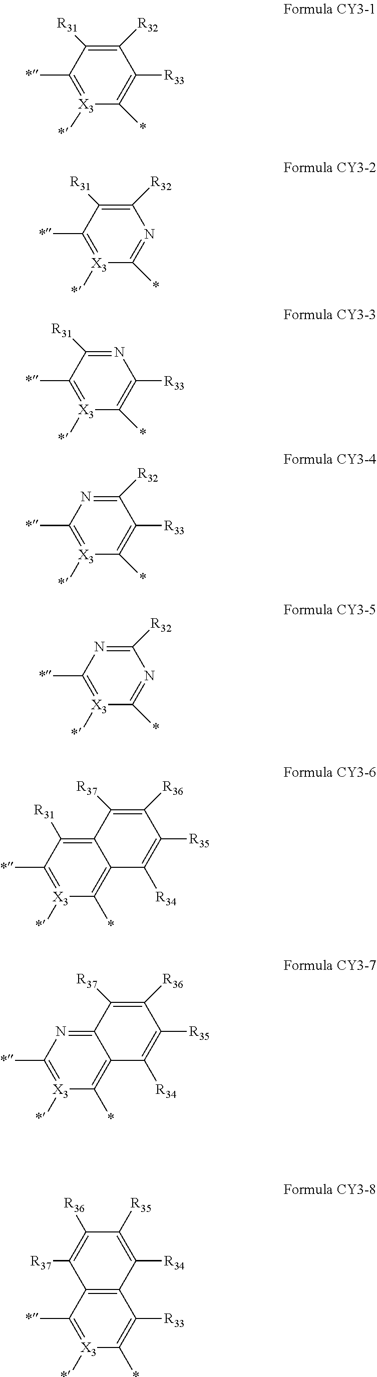 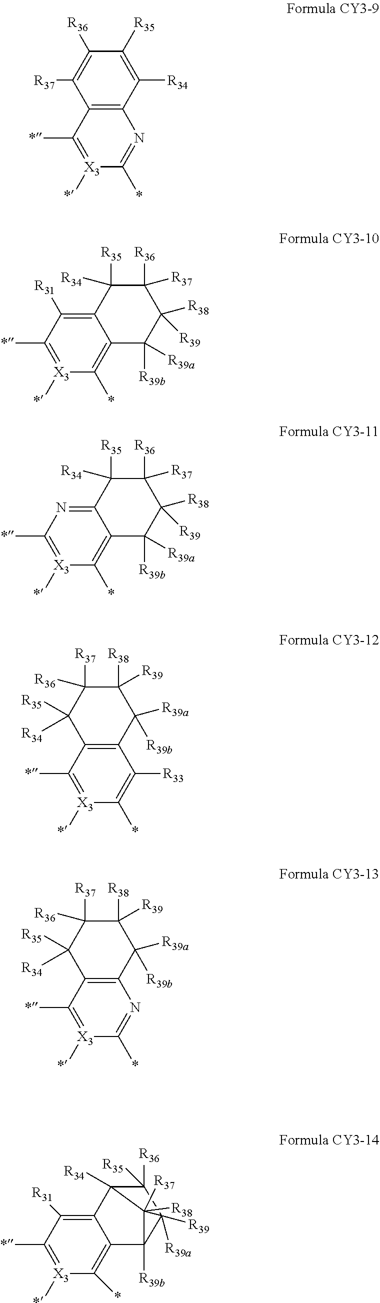 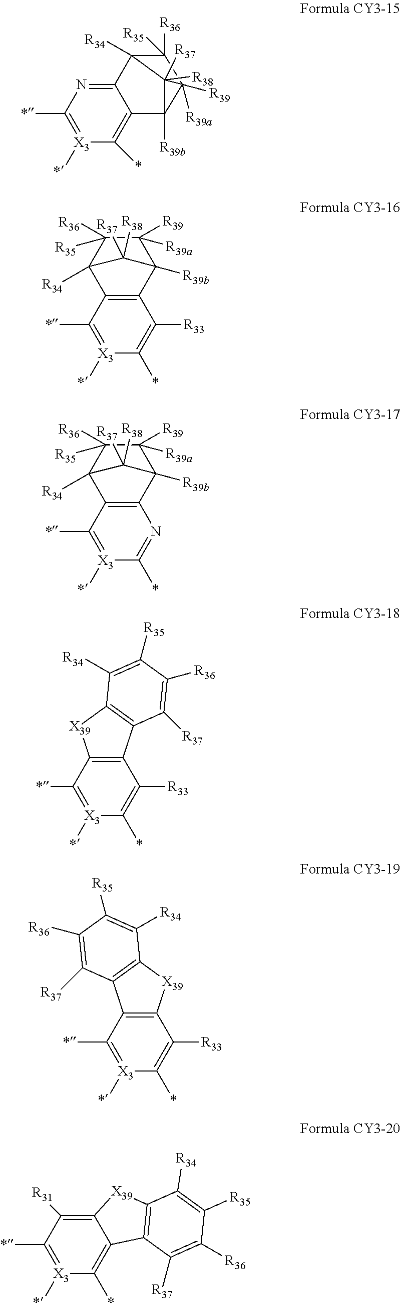 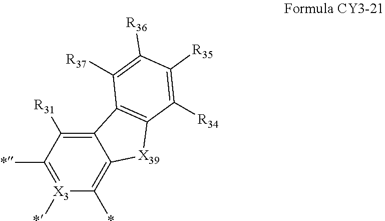
wherein, in Formulae CY3-1 to CY3-21,
X₃ is selected from C and N,
X₃₉ is selected from *—N[(L₃₉)ₐ₃₉-(R₃₉)]—*′, *—B(R₃₉)—*′, *—P(R₃₉)—*′, *—C(R₃₉ₐ)(R_{39b})—*′, *—Si(R₃₉ₐ)(R_{39b})—*′, *—Ge(R₃₉ₐ)(R_{39b})—*′, *—S—*′, *—Se—*′, *—O—*′, *—C(═O)—*′, *—S(═O)—*′, *—S(═O)₂—*′, and *—C(═S)—*′,
L₃₉ and a39 are each the same as described in claim 1 with reference to L₅ and a5, respectively,
R₃₁ to R₃₉, R₃₉ₐ, and R_{39b} are each independently the same as described in claim 1 with reference to R₃, and
*, *′, and *″ each indicate a binding site to an adjacent atom.

11. The organometallic compound of claim 1, wherein a moiety represented by 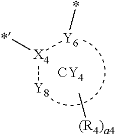
in Formula 1 is selected from groups represented by Formulae CY4-1 to CY4-38: 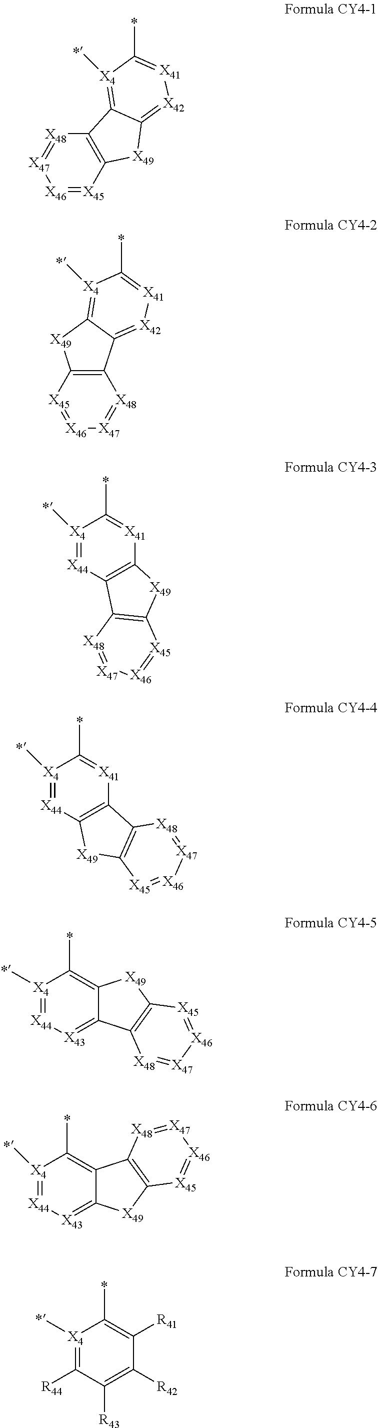 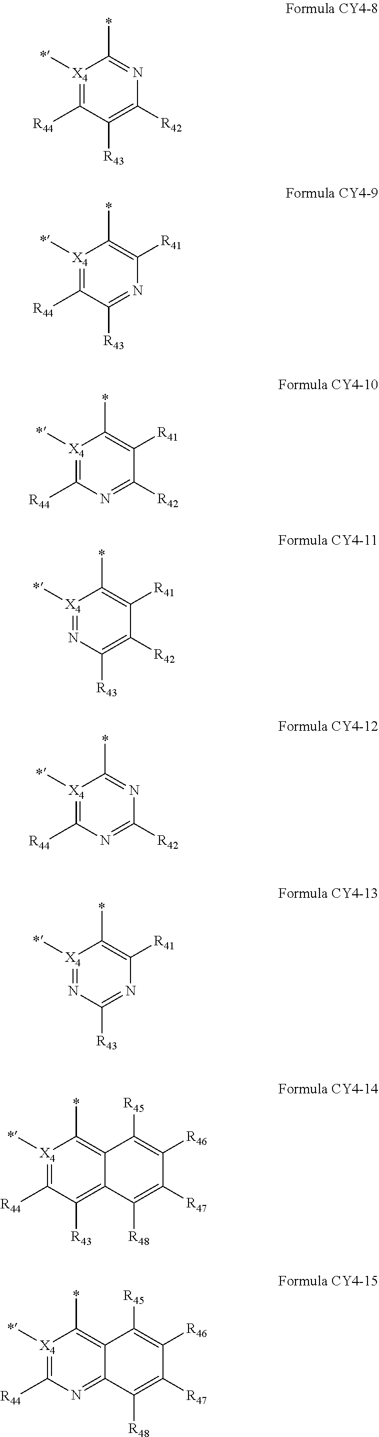 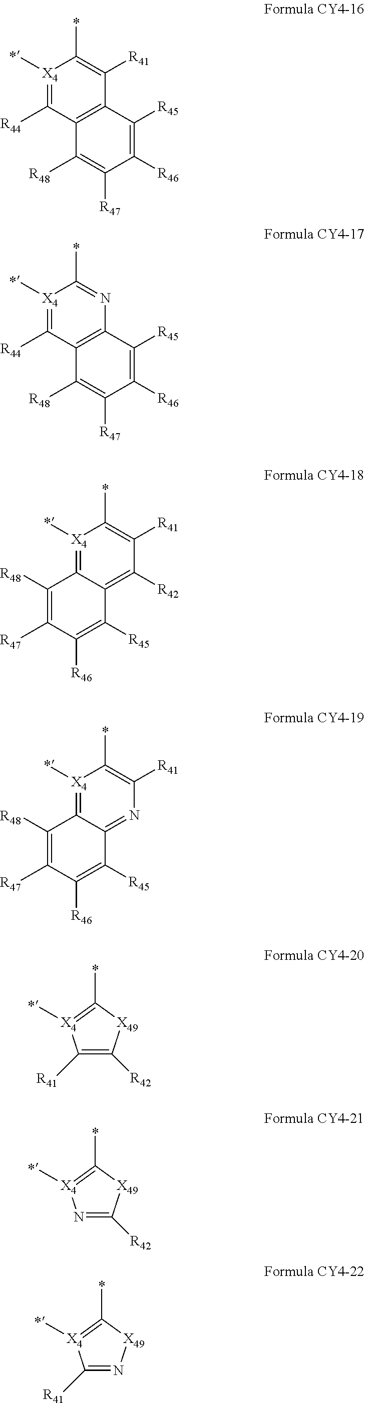 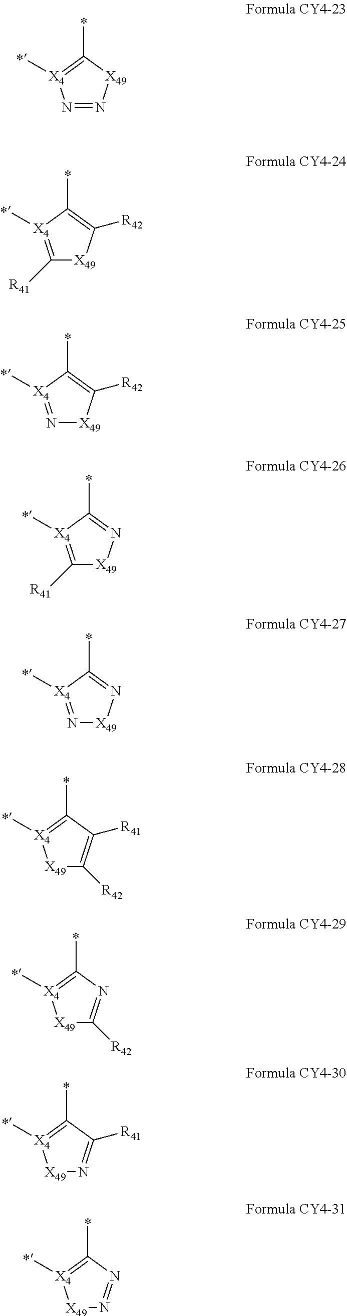 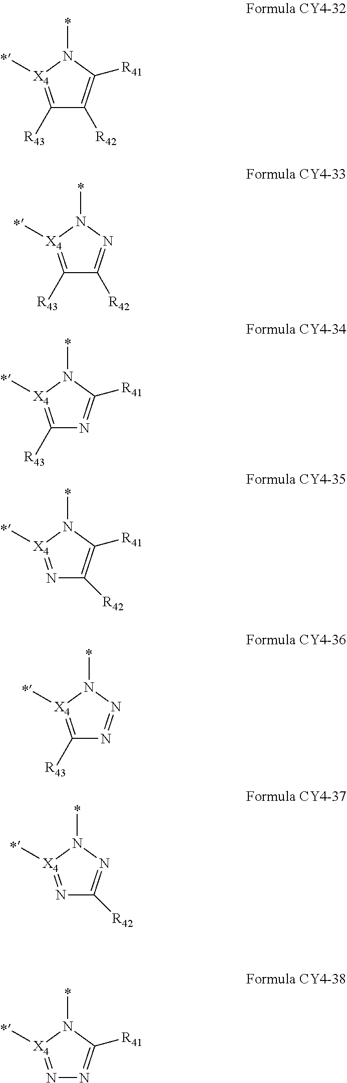
wherein, in Formulae CY4-1 to CY4-38,
X₄ is selected from C and N,
X₄₁ is N or C(R₄₁), X₄₂ is N or C(R₄₂), X₄₃ is N or C(R₄₃), X₄₄ is N or C(R₄₄), X₄₅ is N or C(R₄₅), X₄₆ is N or C(R₄₆), X₄₇ is N or C(R₄₇), X₄₈ is N or C(R₄₈),
X₄₉ is selected from *—N[(L₄₉)ₐ₄₉-(R₄₉)]—*′, *—B(R₄₉)—*′, *—P(R₄₉)—*′, *—C(R₄₉ₐ)(R_{49b})—*′, *—Si(R₄₉ₐ)(R_{49b})—*′, *—Ge(R₄₉ₐ)(R_{49b})—*′, *—S—*′, *—Se—*′, *—O—*′, *—C(═O)—*′, *—S(═O)—*′, *—S(═O)₂—*′, and *—C(═S)—*′,
L₄₉ and a49 are each the same as described in claim 1 with reference to L₅ and a5, respectively,
R₄₁ to R₄₉, R₄₉ₐ, and R_{49b} are each independently the same as described in claim 1 with reference to R₄, and
* and *′ each indicate a binding site to an adjacent atom.

12. The organometallic compound of claim 1, wherein, in Formula 1,
a moiety represented by 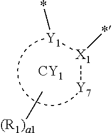
is a group represented by one of Formulae CY1(1) and CY1(2),
a moiety represented by 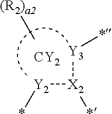
is a group represented by Formulae CY2(1),
a moiety represented by 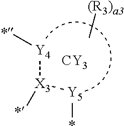
is a group represented by Formula CY3(1), and
a moiety represented by 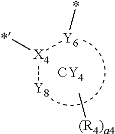
is a group represented by one of Formulae CY4(1) to CY4(7): 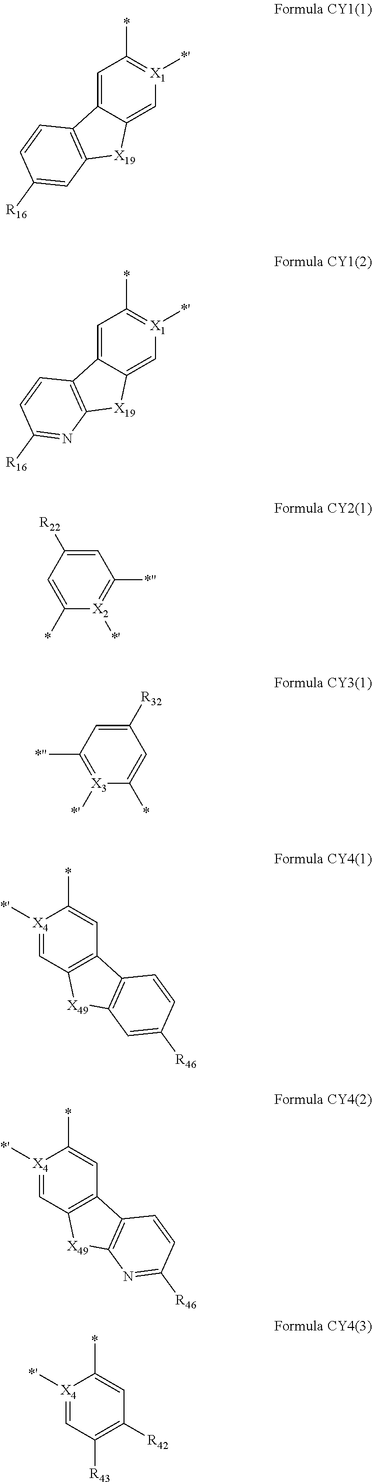 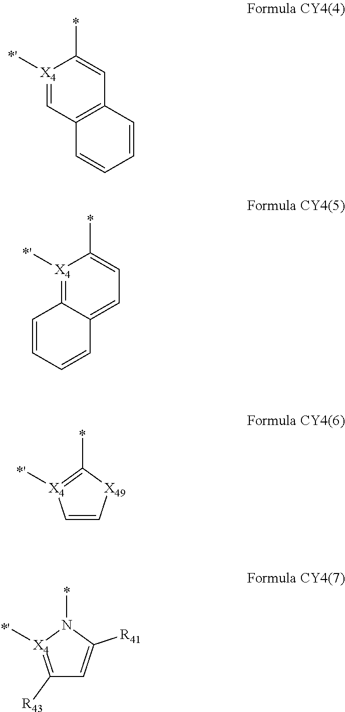
wherein, in Formulae CY1(1), CY1(2), CY2(1), CY3(1), and CY4(1) to CY4(7),
X₁ to X₄ are the same as those described in claim 1,
X₁₉ is selected from *—N[(L₁₉)ₐ₁₉-(R₁₉)]—*′, *—B(R₁₉)—*′, *—P(R₁₉)—*′, *—C(R₁₉ₐ)(R_{19b})—*′, *—Si(R₁₉ₐ)(R_{19b})—*′, *—Ge(R₁₉ₐ)(R_{19b})—*′, *—S—*′, *—Se—*′, *—O—*′, *—C(═O)—*′, *—S(═O)—*′, *—S(═O)₂—*′, and *—C(═S)—*′,
X₄₉ is selected from *—N[(L₄₉)ₐ₄₉-(R₄₉)]—*′, *—B(R₄₉)—*′, *—P(R₄₉)—*′, *—C(R₄₉ₐ)(R_{49b})—*′, *—Si(R₄₉ₐ)(R_{49b})—*′, *—Ge(R₄₉ₐ)(R_{49b})—*′, *—S—*′, *—Se—*′, *—O—*′, *—C(═O)—*′, *—S(═O)—*′, *—S(═O)₂—*′, and *—C(═S)—*′,
L₁₉ and L₄₉ are each the same as described in claim 1 with reference to L₅,
a19 and a49 are each the same as described in claim 1 with reference to a5,
R₁₆, R₁₉, R₁₉ₐ, and R_{19b} are each the same as described in claim 1 with reference to R₁,
R₂₂ is the same as described in claim 1 with reference to R₂,
R₃₂ is the same as described in claim 1 with reference to R₃,
R₄₁ to R₄₃, R₄₆, R₄₉, R₄₉ₐ, and R_{49b} are each the same as described in claim 1 with reference to R₄, and
*, *′, and *″ each indicate a binding site to an adjacent atom.

13. The organometallic compound of claim 1 selected from Compounds 1 to 54:

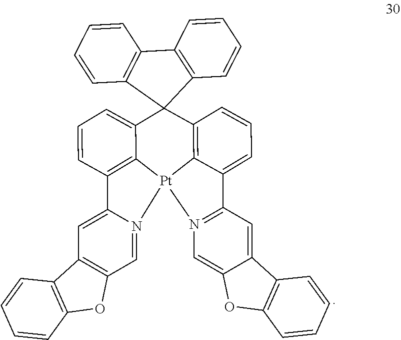

14. An organic light-emitting device comprising:
a first electrode;
a second electrode; and
an organic layer disposed between the first electrode and the second electrode and comprising an emission layer,
wherein the organic layer comprises at least one organometallic compound of claim 1.

15. The organic light-emitting device of claim 14, wherein
the first electrode is an anode,
the second electrode is a cathode, and
the organic layer comprises a hole transport region disposed between the first electrode and the emission layer and an electron transport region disposed between the emission layer and the second electrode,
wherein the hole transport region comprises a hole injection layer, a hole transport layer, an electron blocking layer, or any combination thereof, and
wherein the electron transport region comprises a hole blocking layer, an electron transport layer, an electron injection layer, or any combination thereof.

16. The organic light-emitting device of claim 14, wherein the emission layer comprises the organometallic compound.

17. The organic light-emitting device of claim 16, wherein the emission layer further comprises a host, and an amount of the host in the emission layer is greater than an amount of the organometallic compound in the emission layer.

18. A diagnostic composition that comprises at least one organometallic compound of claim 1.
